SEQUENCE LISTING (229 sequences)

SEQ ID NO: 1 - protein, 5 aa, Artificial Sequence
  Synthetic
NSWMN

SEQ ID NO: 2 - protein, 16 aa, Artificial Sequence
  Synthetic
KSSQSLLFSNGKTYLN

SEQ ID NO: 3 - protein, 19 aa, Artificial Sequence
  Synthetic
QIQLKSDNYATRYVESVRG

SEQ ID NO: 4 - protein, 7 aa, Artificial Sequence
  Synthetic
LVSNLDS

SEQ ID NO: 5 - protein, 4 aa, Artificial Sequence
  Synthetic
GTDY

SEQ ID NO: 6 - protein, 9 aa, Artificial Sequence
  Synthetic
VQGTHFPYT

SEQ ID NO: 7 - protein, 5 aa, Artificial Sequence
  Synthetic
AYNMD

SEQ ID NO: 8 - protein, 11 aa, Artificial Sequence
  Synthetic
RASQDIRNYLN

SEQ ID NO: 9 - protein, 17 aa, Artificial Sequence
  Synthetic
DINPNNGDTVYNQNFKG

SEQ ID NO: 10 - protein, 7 aa, Artificial Sequence
  Synthetic
YTSRLLS

SEQ ID NO: 11 - protein, 15 aa, Artificial Sequence
  Synthetic
RGSYYYGSSHYALDF

SEQ ID NO: 12 - protein, 9 aa, Artificial Sequence
  Synthetic
QQDNSLPRT

SEQ ID NO: 13 - protein, 11 aa, Artificial Sequence
  Synthetic
RASQDISNYLN

SEQ ID NO: 14 - protein, 17 aa, Artificial Sequence
  Synthetic
DINPINGGTIYNQNFEG

SEQ ID NO: 15 - protein, 9 aa, Artificial Sequence
  Synthetic
QQDHTLPRT

SEQ ID NO: 16 - protein, 5 aa, Artificial Sequence
  Synthetic
GYHMN

SEQ ID NO: 17 - protein, 17 aa, Artificial Sequence
  Synthetic
EINPITSGITYNQIFKA

SEQ ID NO: 18 - protein, 9 aa, Artificial Sequence
  Synthetic
RDYGTSLDY

SEQ ID NO: 19 - protein, 5 aa, Artificial Sequence
  Synthetic
NYGVH

SEQ ID NO: 20 - protein, 11 aa, Artificial Sequence
  Synthetic
RASKSIIKYLA

SEQ ID NO: 21 - protein, 16 aa, Artificial Sequence
  Synthetic
VIWRGGSTDYNAAFMS

SEQ ID NO: 22 - protein, 7 aa, Artificial Sequence
  Synthetic
SGSTLQS

SEQ ID NO: 23 - protein, 9 aa, Artificial Sequence
  Synthetic
NYGYDCFAY

SEQ ID NO: 24 - protein, 9 aa, Artificial Sequence
  Synthetic
QQHNEYPLT

SEQ ID NO: 25 - protein, 5 aa, Artificial Sequence
  Synthetic
GYYMN

SEQ ID NO: 26 - protein, 12 aa, Artificial Sequence
  Synthetic
RASKSISKYLAW

SEQ ID NO: 27 - protein, 17 aa, Artificial Sequence
  Synthetic
EINPNTGDPRYDQKFKA

SEQ ID NO: 28 - protein, 7 aa, Artificial Sequence
  Synthetic
RDYGTSL

SEQ ID NO: 29 - protein, 11 aa, Artificial Sequence
  Synthetic
RASKSINKYLA

SEQ ID NO: 30 - protein, 17 aa, Artificial Sequence
  Synthetic
EINPNTGDTNNNQKFKA

SEQ ID NO: 31 - protein, 9 aa, Artificial Sequence
  Synthetic
QQHNEYPIT

SEQ ID NO: 32 - protein, 5 aa, Artificial Sequence
  Synthetic
SYWMD

SEQ ID NO: 33 - protein, 17 aa, Artificial Sequence
  Synthetic
KSSQSLLNSGNQDNYLA

SEQ ID NO: 34 - protein, 17 aa, Artificial Sequence
  Synthetic
NIYPSDSKTHSNQKFRD

SEQ ID NO: 35 - protein, 7 aa, Artificial Sequence
  Synthetic
GASIRES

SEQ ID NO: 36 - protein, 11 aa, Artificial Sequence
  Synthetic
RGGYYGYAMDY

SEQ ID NO: 37 - protein, 9 aa, Artificial Sequence
  Synthetic
QNDHSYPYT

SEQ ID NO: 38 - protein, 5 aa, Artificial Sequence
  Synthetic
TYGVS

SEQ ID NO: 39 - protein, 11 aa, Artificial Sequence
  Synthetic
KASEDIYNRLA

SEQ ID NO: 40 - protein, 16 aa, Artificial Sequence
  Synthetic
VIWGDGTTNYHSALKP

SEQ ID NO: 41 - protein, 7 aa, Artificial Sequence
  Synthetic
GATNLVT

SEQ ID NO: 42 - protein, 9 aa, Artificial Sequence
  Synthetic
YYGYPFFAY

SEQ ID NO: 43 - protein, 9 aa, Artificial Sequence
  Synthetic
QQYWSSPPT

SEQ ID NO: 44 - protein, 5 aa, Artificial Sequence
  Synthetic
SYGVS

SEQ ID NO: 45 - protein, 10 aa, Artificial Sequence
  Synthetic
RASSTVNYMY

SEQ ID NO: 46 - protein, 16 aa, Artificial Sequence
  Synthetic
VIWGDGSTDYHSTLTS

SEQ ID NO: 47 - protein, 7 aa, Artificial Sequence
  Synthetic
YTSRLAP

SEQ ID NO: 48 - protein, 10 aa, Artificial Sequence
  Synthetic
FYGYDEGFAY

SEQ ID NO: 49 - protein, 9 aa, Artificial Sequence
  Synthetic
QQFTSSPLT

SEQ ID NO: 50 - protein, 115 aa, Artificial Sequence
  Synthetic
EVKLEESGGGLVQPGGSMKLSCFVSGFTFSNSWMNWVRQSPEKGLEWVAQIQLKSDNYATRYVESVRGRFIISRDDSKSSVSLQMNNLRTEDTGIYYCSSGTDYWGQGTTLTVSS

SEQ ID NO: 51 - DNA, 345 bp, Artificial Sequence
  Synthetic
gaagtgaagcttgaggagtctggaggaggcttggtgcaacctggaggatccatgaaactctcctgttttgtctctggattcactttcagtaactcctggatgaactgggtccgccagtctccagagaaggggcttgagtgggttgctcagattcaattgaaatctgataattatgcaacacgttatgtggagtctgtgagagggaggttcatcatctcaagagatgattccaaaagtagtgtctccctgcaaatgaacaacttaaggactgaagacactggaatttattactgttcctctgggaccgactactggggccaaggcaccactctcacagtctcctca

SEQ ID NO: 52 - protein, 112 aa, Artificial Sequence
  Synthetic
DVVMTQTPLALSVIIGQPASISCKSSQSLLFSNGKTYLNWLLQRPGQSPKRLIYLVSNLDSGVPDRFTGSGSGTDFTLKIGRVEAEDLGVYYCVQGTHFPYTFGGGTKLEIK

SEQ ID NO: 53 - DNA, 336 bp, Artificial Sequence
  Synthetic
gatgttgtgatgacccagactccactcgctttgtcggttatcattggacaaccagcctctatctcttgcaagtcaagtcagagcctcttatttagtaatggaaaaacctatttgaattggttattacagaggccaggccagtctccaaagcgcctaatctatctggtgtctaatctggactctggagtccctgacaggttcactggcagtggatcaggaacagattttacactgaaaatcggcagagtggaggctgaagatttgggagtttattactgcgtgcaaggtacacattttccatacacgttcggtggggggaccaagttggaaataaaa

SEQ ID NO: 54 - protein, 124 aa, Artificial Sequence
  Synthetic
EVQLQQSGPELVKPGASVKIPCKASGYTFTAYNMDWVKQSHGMSLEWIGDINPNNGDTVYNQNFKGKATLTVDKSSSTAYMELRSLTSEDTAVYYCARRGSYYYGSSHYALDFWGQGTSVSVSS

SEQ ID NO: 55 - DNA, 372 bp, Artificial Sequence
  Synthetic
gaggtccagctgcaacagtctggacctgagctggtgaagcctggggcttcagtgaagataccctgcaaggcttctggatacacattcactgcctacaacatggactgggtgaagcagagccatggaatgagccttgagtggattggagatattaatcctaacaatggtgatactgtctacaaccagaatttcaagggcaaggccacattgactgtagacaagtcctccagcacagcctacatggaactccgcagcctgacatctgaggacactgcagtctattactgtgcaagaagggggtcctattactacggtagtagtcactatgctctggacttctggggtcaaggaacctcagtctccgtctcctca

SEQ ID NO: 56 - protein, 107 aa, Artificial Sequence
  Synthetic
DIQMTQITSSLSASLGNRVTISCRASQDIRNYLNWYQQKPDGTVKLLIYYTSRLLSGVPSRFSGSGSGTDYSLTISNLEQEDIATYFCQQDNSLPRTFGGGTRLEIR

SEQ ID NO: 57 - DNA, 321 bp, Artificial Sequence
  Synthetic
gatatccagatgacacagattacatcctccctgtctgcctctctgggaaacagagtcaccatcagttgcagggcaagtcaggacattaggaattatttaaactggtatcagcagaaaccagatggaactgttaaactcctgatctactacacatcaagattactctcaggagtcccatcacggttcagtggcagtgggtctggaacagattattctctcaccattagcaacctggagcaagaagatattgccacttacttttgccaacaggataattcgcttcctcggacgttcggtggaggcaccaggctggaaatcaga

SEQ ID NO: 58 - protein, 124 aa, Artificial Sequence
  Synthetic
EVQLQQSGPELVKPGASVKIPCKASGYTFTAYNMDWVKQSHGMSLEWIGDINPINGGTIYNQNFEGKATLTVDKSSSTAYLELRSLTSEDTAVYYCARRGSYYYGSSHYALDFWGQGTSVSVSS

SEQ ID NO: 59 - DNA, 372 bp, Artificial Sequence
  Synthetic
gaggtccagctgcaacagtctggacctgagctggtgaagcctggggcttcagtgaagataccctgcaaggcttctggatacacattcactgcctacaacatggactgggtgaagcagagccatggaatgagccttgagtggattggagatattaatcctatcaatggtggtactatctacaaccagaatttcgagggcaaggccacattgactgtagacaagtcttccagcacagcctacttggaactccgcagcctgacatctgaggacactgcagtctattactgtgcaagaagggggtcctattactacggtagtagccactatgctttggacttctggggtcaaggaacctcagtctccgtctcctca

SEQ ID NO: 60 - skipped

SEQ ID NO: 61 - DNA, 321 bp, Artificial Sequence
  Synthetic
gatatccagatgacacagattacatcctccctgtctgcctctctgggagacagagtcaccatcacttgcagggcaagtcaggacattagcaattatttaaactggtatcagcagaaaccagatggaactgttaaactcctgatctactacacatcaagattactctcaggagtcccatcaaggttcagtggcagtgggtctggaacagattattctctcaccattagcaacctggagcaagaagatattgccacttacttttgccaacaggatcatacgcttcctcggacgttcggtggaggcaccaagctggaaatcaga

SEQ ID NO: 62 - protein, 118 aa, Artificial Sequence
  Synthetic
EVQLRQSGPELVKPGAAVKISCKASGYSFTGYHMNWVKQSPEKSLEWIGEINPITSGITYNQIFKAKATLTVDKSSSTAYLQLKSLTSEDSAVYYCTRRDYGTSLDYWGQGTTLTVSS

SEQ ID NO: 63 - DNA, 354 bp, Artificial Sequence
  Synthetic
gaggtccagctgcggcagtctggacctgagctggtgaagcctggggctgcggtgaagatatcctgcaaggcttctggttactcattcactggctaccacatgaactgggtgaagcaaagtcctgaaaagagccttgagtggattggagagattaatcctatcactagtggtattacctacaaccagattttcaaggccaaggccacattgactgtagacaaatcctccagcacagcctacttgcagctcaagagcctgacatctgaggactctgcagtctattactgtacaagaagggactacggtactagccttgactactggggccaaggcaccactctcacagtctcctca

SEQ ID NO: 64 - protein, 107 aa, Artificial Sequence
  Synthetic
DIQMTQITSSLSASLGDRVTITCRASQDISNYLNWYQQKPDGTVKLLIYYTSRLLSGVPSRFSGSGSGTDYSLTISNLEQEDIATYFCQQDHTLPRTFGGGTKLEIR

SEQ ID NO: 65 - DNA, 321 bp, Artificial Sequence; identical to SEQ ID NO: 61
  Synthetic

SEQ ID NO: 66 - protein, 117 aa, Artificial Sequence
  Synthetic
QVQLKQSGPGLVQPSQSLSITCTVSGFSLTNYGVHWIRQSPGKGLEWLGVIWRGGSTDYNAAFMSRLSITKDNSKSQVFFQMNSLQAADTAIYYCANNYGYDCFAYWGQGTLVTVSA

SEQ ID NO: 67 - DNA, 351 bp, Artificial Sequence
  Synthetic
caggtgcagctgaagcagtcaggacctggcctagtgcagccctcacagagcctgtccataacctgcacagtctctggtttctcattaactaactatggtgtacactggattcgccagtctccaggaaagggtctggagtggctgggagtgatatggagaggtggaagcacagactacaatgcagctttcatgtccagactgagcatcaccaaggacaactccaagagccaagttttctttcaaatgaacagtctgcaagctgctgacactgccatatattactgtgccaacaactatggttacgactgctttgcttactggggccaagggactctggtcactgtctctgca

SEQ ID NO: 68 - protein, 107 aa, Artificial Sequence
  Synthetic
DVQITQSPSYLAASPGETITINCRASKSIIKYLAWYQEKPGKTNKLLIYSGSTLQSGIPSRFSGSGSGTDFTLTITSLEPEDFAMYYCQQHNEYPLTFGAGTRLELK

SEQ ID NO: 69 - DNA, 321 bp, Artificial Sequence
  Synthetic
gatgtccaaataacccagtctccatcttatcttgctgcatctcctggagaaaccattactattaattgcagggcaagtaagagtattatcaaatatttagcctggtatcaagagaaacctgggaaaactaataagcttcttatctactctggatccactttgcaatctggaattccatcaaggttcagtggcagtggatctggtacagatttcactctcaccatcactagcctggagcctgaagattttgcaatgtattactgtcaacagcataatgaatacccgctcacgttcggtgctgggaccaggctggagctgaaa

SEQ ID NO: 70 - protein, 118 aa, Artificial Sequence
  Synthetic
EVQLQQSGPGLVKPGASVKISCRASGYLFTGYYMNWVKQSPEKSLEWIGEINPNTGDPRYDQKFKAKATLTVDRSSSTAYMHLKSLTSEDSAVYFCARRDYGTSLDYWGQGTTLTVTS

SEQ ID NO: 71 - DNA, 354 bp, Artificial Sequence
  Synthetic
gaggttcagctgcagcagtctggacctgggctggtgaagcctggggcttcagtgaagatatcctgcagggcttctggttacctattcactggctactacatgaactgggtgaagcaaagtcctgaaaagagccttgagtggattggagagattaatcctaacactggtgatcctagatacgaccagaagtttaaggccaaggccacattgactgtagacagatcctccagcacagcctacatgcacctcaagagcctgacatctgaagactctgcagtctatttctgtgccaggagggactacggtactagccttgactactggggccaaggcaccactctcacagtcacctca

SEQ ID NO: 72 - protein, 107 aa, Artificial Sequence
  Synthetic
DVQITQSPSYLAASPGETITINCRASKSISKYLAWYQEKPGKTNKLLIYSGSTLQSGIPSRFSGSGSGSDFTLTISSLEPEDFAMYYCQQHNEYPLTFGAGTKLELK

SEQ ID NO: 73 - DNA, 321 bp, Artificial Sequence
  Synthetic
gatgtccagataacccagtctccatcttatcttgctgcatctcctggagaaaccattactattaattgcagggcaagtaagagcattagcaaatatttagcctggtatcaagagaaacctgggaaaactaataagcttcttatctactctggatccactttgcaatctggaattccatcaaggttcagtggcagtggatctggttcagatttcactctcaccatcagtagcctggagcctgaagattttgcaatgtattactgtcaacagcataatgaatatccgctcacgttcggtgctgggaccaagctggagctgaaa

SEQ ID NO: 74 - protein, 118 aa, Artificial Sequence
  Synthetic
EVQLQQSGPELVKPGASVKISCKATGYLFTGYYMNWVKQSPEKSLEWIGEINPNTGDTNNNQKFKAKATMTVDRSSSTAYMQLKRLTSEDSAVYYCVRRDYGTSLDYWGQGTTLTVSS

SEQ ID NO: 75 - DNA, 354 bp, Artificial Sequence
  Synthetic
gaggttcagctgcagcagtctggacctgagttggtgaagcctggggcttcagtgaagatatcctgcaaggctactggttacttattcactggctactacatgaactgggtgaaacaaagtcctgaaaagagccttgagtggattggagagatcaatcctaacactggtgatactaacaacaaccagaagttcaaggccaaggccacaatgactgttgacagatcctccagcacagcctacatgcagctcaagaggctgacatctgaggactctgcagtctattattgtgtaagaagggactacggtactagtcttgactactggggccaaggcaccactctcacagtctcctca

SEQ ID NO: 76 - protein, 107 aa, Artificial Sequence
  Synthetic
DVQITQSPSYLAASPGETITINCRASKSINKYLAWYQEKPGKTNKLLIHSGSTLQSGIPSRFSGSRSGTDFTLTISNLEPEDFAMYYCQQHNEYPITFGAGTKLELK

SEQ ID NO: 77 - DNA, 321 bp, Artificial Sequence
  Synthetic
gatgtccagataacccagtctccatcttatcttgctgcatctcctggagaaaccattactattaattgcagggcaagtaagagtattaacaaatatttagcctggtatcaagagaaacctgggaaaactaataagcttcttatccactctggatccactttgcaatctggaattccatcaaggttcagtggcagtagatctggaacagatttcactctcaccatcagtaacctggagcctgaagattttgcaatgtattactgtcaacaacataatgaatatccgatcacgttcggtgctgggaccaagctggagctgaaa

SEQ ID NO: 78 - protein, 120 aa, Artificial Sequence
  Synthetic
QVQLQQPGAELVRPGSSVKLSCKTSDYTFTSYWMDWVKQRPGQGLEWIGNIYPSDSKTHSNQKFRDKATLTVDKSSSTAYMQLNSLTSEDSAVYYCARRGGYYGYAMDYWGQGTSVTVSS

SEQ ID NO: 79 - DNA, 360 bp, Artificial Sequence
  Synthetic
caggtccaactgcagcagcctggggctgaactggtgaggcctgggtcttcagtgaagctgtcctgcaagacttctgactacaccttcaccagctactggatggattgggtgaagcagaggcctggacaaggccttgaatggattggtaacatttacccttctgatagtaaaactcactccaatcaaaagttcagggacaaggccacattgactgtagacaaatcctccagcacagcctacatgcagctcaacagcctgacatctgaagactctgcggtctattactgtgcaagaagaggtggttactacggctatgctatggactactggggtcaaggaacctcagtcaccgtctcctca

SEQ ID NO: 80 - protein, 113 aa, Artificial Sequence
  Synthetic
DIVMTQSPSSLSVSAGEKVTMSCKSSQSLLNSGNQDNYLAWYQQKPGQPPKLLIYGASIRESGVPDRFTGSGSGTDFTLTISSVQAEDLAVYYCQNDHSYPYTFGGGTKLEIK

SEQ ID NO: 81 - DNA, 339 bp, Artificial Sequence
  Synthetic
gacattgtgatgacacagtctccatcctccctgagtgtgtcagcaggagagaaggtcactatgagctgcaagtccagtcagagtctgttaaatagtggaaatcaagacaactacttggcctggtaccagcagaaaccagggcagcctcctaaactgttgatctacggggcatccattagggaatctggggtccctgatcgcttcacaggcagtggatctggaaccgatttcactcttaccatcagtagtgtgcaggctgaagacctggcagtttattactgtcaaaatgatcatagttatccgtacacgttcggaggggggaccaagttggaaataaaa

SEQ ID NO: 82 - protein, 117 aa, Artificial Sequence
  Synthetic
QVQLKESGPGLVAPSQSLSITCTVSGFSLITYGVSWVRQPPGKGLEWLGVIWGDGTTNYHSALKPRLTISKDDSKSQVLLKLNSLQTDDTATYYCANYYGYPFFAYWGQGTLVTVSA

SEQ ID NO: 83 - DNA, 351 bp, Artificial Sequence
  Synthetic
caggtgcagctgaaggagtcaggacctggcctggtggcgccctcacagagcctgtccatcacatgcactgtctcaggattctcattaatcacctatggtgtaagctgggttcgccagcctccaggaaagggtctggagtggctgggagtaatatggggtgacgggaccacaaattatcattcagctctcaaacccagactgaccatcagcaaggatgactccaagagccaagtcctcttaaaattgaacagtctgcaaactgatgacacagccacgtactactgtgccaactactatggttaccccttttttgcttactggggccaagggactctggtcactgtctctgca

SEQ ID NO: 84 - protein, 107 aa, Artificial Sequence
  Synthetic
DIQMTQSSSSFSVSLGDRVTITCKASEDIYNRLAWYQQKPGSAPGVLISGATNLVTGIPSRFSGSGSGKDYTLTITSLQTEDVATYYCQQYWSSPPTFGGGTKLEIK

SEQ ID NO: 85 - DNA, 321 bp, Artificial Sequence
  Synthetic
gacatccagatgacacaatcttcatcctccttttctgtatctctaggagacagagtcaccattacttgcaaggcaagtgaggacatatataatcggttagcctggtatcaacagaaaccaggaagtgctcctggggtcttaatatctggtgcaaccaatttggtaactgggattccttcaagattcagtggcagtggatctggaaaggattacactctcaccattaccagtcttcagactgaagatgttgctacttattactgtcaacagtattggagtagtcctccgacgttcggtggaggcaccaagctggaaatcaaa

SEQ ID NO: 86 - protein, 118 aa, Artificial Sequence
  Synthetic
QVQLKESGPGLVAPLQSLSITCTVSGFSLSSYGVSWVRQLPGKGLEWLGVIWGDGSTDYHSTLTSRLSFSKDNSESQVFLKLNSLQTDDTATYYCAIFYGYDEGFAYWGQGTLVTVSA

SEQ ID NO: 87 - DNA, 354 bp, Artificial Sequence
  Synthetic
caggtgcagctgaaggagtcaggacctggcctggtggcgcccttacagagcctgtccatcacatgcactgtctcagggttctcattaagcagttatggtgtaagctgggttcgccagcttccaggaaagggtctggagtggctgggagtaatatggggtgacgggagcacagattatcattcaactctcacatccagactgagcttcagcaaggataattccgagagccaagttttcttaaaactgaatagtctgcaaactgatgacacagccacatactactgtgccattttctatggttacgacgaagggtttgcttattggggccaagggactctggtcactgtctctgca

SEQ ID NO: 88 - protein, 106 aa, Artificial Sequence
  Synthetic
ENVLTQSPAIMSAFLGEKVTMSCRASSTVNYMYWYQQKSDASPKVWIYYTSRLAPGVPARFSGSGSGNSFSLTISSMEGEDAATYYCQQFTSSPLTFGAGTKLELR

SEQ ID NO: 89 - DNA, 318 bp, Artificial Sequence
  Synthetic
gaaaatgtgctcacccagtctccagcaatcatgtctgcatttctaggggagaaggtcaccatgagctgcagggccagctcaactgtaaattacatgtactggtaccagcagaagtcagatgcctcccccaaagtttggatttattacacatccaggttggctcctggagtcccagctcgcttcagtggcagtgggtctgggaactctttttctctcacaatcagcagcatggagggtgaagatgctgccacttattactgccagcagtttactagttccccgctcacgttcggtgctgggaccaagctggagctgaga

SEQ ID NO: 90 - protein, 5 aa, Artificial Sequence
  Synthetic
GGGGS

SEQ ID NO: 91 - protein, 10 aa, Artificial Sequence
  Synthetic
GGGGSGGGGS

SEQ ID NO: 92 - protein, 15 aa, Artificial Sequence
  Synthetic
GGGGSGGGGSGGGGS

SEQ ID NO: 93 - protein, 20 aa, Artificial Sequence
  Synthetic
GGGGSGGGGSGGGGSGGGGS

SEQ ID NO: 94 - protein, 244 aa, Artificial Sequence
  Synthetic
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 95 - protein, 5 aa, Artificial Sequence
  Synthetic
SYGVN

SEQ ID NO: 96 - protein, 16 aa, Artificial Sequence
  Synthetic
MIWSGGNTDYNAAFIS

SEQ ID NO: 97 - protein, 8 aa, Artificial Sequence
  Synthetic
IWYYGMDY

SEQ ID NO: 98 - protein, 11 aa, Artificial Sequence
  Synthetic
KASQNVGTAVA

SEQ ID NO: 99 - protein, 7 aa, Artificial Sequence
  Synthetic
SASIRYP

SEQ ID NO: 100 - protein, 8 aa, Artificial Sequence
  Synthetic
LQYITFPR

SEQ ID NO: 101 - protein, 116 aa, Artificial Sequence
  Synthetic
QIQLKQSGPGLVQPSQSLSITCTVSGFSLSSYGVNWVRQSPGKGLEWLGMIWSGGNTDYNAAFISRVSFTKDNSKSQVFFTMNSLQADDTAIYYCARIWYYGMDYWGQGTSVTVSS

SEQ ID NO: 102 - DNA, 348 bp, Artificial Sequence
  Synthetic
cagatacagctgaagcagtcaggacctggcctagtgcagccctcacagagtctgtccatcacctgcacagtctctggtttctcattaagtagttatggtgtaaattgggttcgccagtctccaggaaagggtctggaatggctgggaatgatatggagtggtggaaacacagactataatgcagctttcatatccagagtgagcttcaccaaggacaattccaagagccaagttttctttacaatgaacagtctgcaagctgatgacacagccatatattactgtgccagaatatggtactatggtatggactactggggtcagggaacctcagtcaccgtctcctca

SEQ ID NO: 103 - protein, 107 aa, Artificial Sequence
  Synthetic
DIVMTQSQKFMSTTVGDRVSITCKASQNVGTAVAWFQQKPGQSPKILIYSASIRYPGVPDRFTGSGSGTDFTLTTSNVQSEDLADYFCLQYITFPRTFGGGTKLYIK

SEQ ID NO: 104 - protein, 8 aa, Artificial Sequence
  Synthetic
DYTFTSYW

SEQ ID NO: 105 - protein, 8 aa, Artificial Sequence
  Synthetic
IYPSDSKT

SEQ ID NO: 106 - protein, 13 aa, Artificial Sequence
  Synthetic
ARRGGYYAYAMDY

SEQ ID NO: 107 - protein, 12 aa, Artificial Sequence
  Synthetic
QSLLNSGNQDNY

SEQ ID NO: 108 - protein, 3 aa, Artificial Sequence
  Synthetic
GAR

SEQ ID NO: 109 - protein, 120 aa, Artificial Sequence
  Synthetic
QVQLLQPGAELVRPGSSVKLSCKASDYTFTSYWMDWVKQRPGQGLEWIGNIYPSDSKTHYNQKFKDKATLTVDKSSSTAYMQLNSLTSEDSAVYYCARRGGYYAYAMDYWGQGTSVTVSS

SEQ ID NO: 110 - DNA, 321 bp, Artificial Sequence
  Synthetic
gacattgtgatgacccagtctcaaaaattcatgtccacaacagtaggagacagggtcagcatcacctgcaaggccagtcagaatgtgggtactgctgtagcctggtttcaacagaaaccaggacaatctcctaaaatactgatttattcagcatccattcgctaccctggagtccctgatcgcttcacaggcagtggatctgggacagatttcactctcaccactagcaatgtgcagtctgaagacctggcagattatttctgtctgcaatatatcacctttcctcggacgttcggtggaggcaccaagctgtatatcaaa

SEQ ID NO: 111 - protein, 113 aa, Artificial Sequence
  Synthetic
DIVMTQSPSSLSVSAGEKVTMRCKSSQSLLNSGNQDNYLAWYQQKPGQPPKLLLYGARIRESGVPDRFIGSGSGTDFTLTISSVQAEDLAVYYCQNDHSYPYTFGGGTKVEIK

SEQ ID NO: 112 - skipped

SEQ ID NO: 113 - protein, 5 aa, Artificial Sequence
  Synthetic
SNTMH

SEQ ID NO: 114 - protein, 17 aa, Artificial Sequence
  Synthetic
YINPGSAYTNYNQKFND

SEQ ID NO: 115 - protein, 14 aa, Artificial Sequence
  Synthetic
LATYYDNDGYAMDY

SEQ ID NO: 116 - protein, 7 aa, Artificial Sequence
  Synthetic
YTSKLHS

SEQ ID NO: 117 - protein, 9 aa, Artificial Sequence
  Synthetic
QQGHTLPWT

SEQ ID NO: 118 - protein, 123 aa, Artificial Sequence
  Synthetic
QVQLQQSGAELARPGASVKMSCKASGYTFTSNTMHWVKQRPGQGLEWIGYINPGSAYTNYNQKFNDKATLTADKSSSTASMQLSSLTSEDSAVYYCARLATYYDNDGYAMDYWGQGTSVTVSS

SEQ ID NO: 119 - skipped

SEQ ID NO: 120 - protein, 107 aa, Artificial Sequence
  Synthetic
DIQMTQSTSSLSASLGDRVTISCRASQDISNYLNWYQQKPDGTVKLLIYYTSKLHSGVPSRFSGSGSGTDFSLTITNLEQEDFATYFCQQGHTLPWTFGGGTKLEIK

SEQ ID NO: 121 - skipped

SEQ ID NO: 122 - protein, 118 aa, Artificial Sequence
  Synthetic
EVQLQQSGTGLVKPGTSVKLSCKASGYSFTSYWITWVKQRPGQGLEWIGDISPAGGGRNYNERFKNKATLTVDASSSTAYMQLSSLTSEDSAVYYCVRGDSTVDLDYWGQGTTLTVSS

SEQ ID NO: 123 - skipped

SEQ ID NO: 124 - protein, 112 aa, Artificial Sequence
  Synthetic
DIVMTQAAPSVPVTPGESVSISCRSSKSLLHSNGNTYLYWFLQRPGQSPQLLIYRVSNLASGVPDRFSGSGSGTAFTLRISRVEAEDVGVYYCMQHLEYPYTFGGGTKLEIK

SEQ ID NO: 125 - skipped

SEQ ID NO: 126 - protein, 5 aa, Artificial Sequence
  Synthetic
DYNMD

SEQ ID NO: 127 - protein, 17 aa, Artificial Sequence
  Synthetic
DINPHNGDTIYNQKFKG

SEQ ID NO: 128 - protein, 15 aa, Artificial Sequence
  Synthetic
RGAYYYGSSHYALDF

SEQ ID NO: 129 - protein, 11 aa, Artificial Sequence
  Synthetic
RASQDITNYLN

SEQ ID NO: 130 - protein, 7 aa, Artificial Sequence
  Synthetic
YTSRLFS

SEQ ID NO: 131 - protein, 8 aa, Artificial Sequence
  QQGNTLPW
QQGNTLPW

SEQ ID NO: 132 - protein, 124 aa, Artificial Sequence
  Synthetic
EVLLQQSGPELVKPGASVKIPCKASGYGFTDYNMDWIKQSHGKSLEWIGDINPHNGDTIYNQKFKGKAKLTVDKSSTTAYMELRSLASEDTAVYYCARRGAYYYGSSHYALDFWGQGTSVTVSS

SEQ ID NO: 133 - skipped

SEQ ID NO: 134 - protein, 107 aa, Artificial Sequence
  Synthetic
DIQMTQTTSSLSASLGDRVTISCRASQDITNYLNWYQQKPDGTVKLLIYYTSRLFSGVPSRFSGSGSGTDYSLTISNLKREDIATYFCQQGNTLPWTFGGGTKLEIK

SEQ ID NO: 135 - skipped

SEQ ID NO: 136 - protein, 17 aa, Artificial Sequence
  Synthetic
EINPNKGDSNLNQNFKA

SEQ ID NO: 137 - protein, 9 aa, Artificial Sequence
  Synthetic
RDYGTSLDH

SEQ ID NO: 138 - protein, 11 aa, Artificial Sequence
  Synthetic
RASKSISKYLA

SEQ ID NO: 139 - protein, 7 aa, Artificial Sequence
  Synthetic
SGSTLHT

SEQ ID NO: 140 - protein, 8 aa, Artificial Sequence
  Synthetic
QQHNEYPI

SEQ ID NO: 141 - protein, 118 aa, Artificial Sequence
  Synthetic
EVQLQQSGPELVKPGASVKISCKASGYLFTGYYMNWVKQSPEKSLEWIGEINPNKGDSNLNQNFKAKATLTVDKSSSTAYMQFKSLTFEDSAVYFCVRRDYGTSLDHWGQGTTLTVSS

SEQ ID NO: 142 - skipped

SEQ ID NO: 143 - protein, 107 aa, Artificial Sequence
  Synthetic
DVQITQSPSYLATSPGETITINCRASKSISKYLAWYQEKPGKTKKLLIYSGSTLHTAIPSRFSGRGFGTDFTLTISSLEPEDFAMYYCQQHNEYPITFGGGTKLELK

SEQ ID NO: 144 - skipped

SEQ ID NO: 145 - protein, 7 aa, Artificial Sequence
  Synthetic
SGSTLHS

SEQ ID NO: 146 - protein, 118 aa, Artificial Sequence
  Synthetic
EVQLQQSGPELVKPGASVKISCKATGYLFTGYYMNWVKQSPEKSLEWIGEINPNTGDTNNNQKFKAKATMTVDRSSSTAYMQLKSLTSEDSAVYYCVRRDYGTSLDYWGQGTTLTVSS

SEQ ID NO: 147 - skipped

SEQ ID NO: 148 - protein, 107 aa, Artificial Sequence
  Synthetic
DVQITQSPSYLAASPGETITINCRASKSINKYLAWYQEKPGKTNKLLIHSGSTLHSGIPSRFSGSRSGTDFTLTISNLEPEDFAMYYCQQHNEYPLTFGAGTKLELK

SEQ ID NO: 149 - skipped

SEQ ID NO: 150 - protein, 8 aa, Artificial Sequence
  Synthetic
GYTFTSNT

SEQ ID NO: 151 - protein, 8 aa, Artificial Sequence
  Synthetic
INPGSAYT

SEQ ID NO: 152 - protein, 16 aa, Artificial Sequence
  Synthetic
ARLATYYDNDGYAMDY

SEQ ID NO: 153 - protein, 5 aa, Artificial Sequence
  Synthetic
SSVNY

SEQ ID NO: 154 - protein, 3 aa, Artificial Sequence
  Synthetic
GTS

SEQ ID NO: 155 - protein, 9 aa, Artificial Sequence
  Synthetic
FQGSGYPYT

SEQ ID NO: 156 - protein, 123 aa, Artificial Sequence; identical to SEQ ID NO: 118
  Synthetic

SEQ ID NO: 157 - skipped

SEQ ID NO: 158 - protein, 106 aa, Artificial Sequence
  Synthetic
ENVLTQSPAIMSASPGEKVTMTCSASSSVNYMYWYQQRSSTSPKLWIYGTSKLASGVPGRFSGSGSGNSYSLTISSMEAEDVATYYCFQGSGYPYTFGGGTKLEIK

SEQ ID NO: 159 - skipped

SEQ ID NO: 160 - protein, 8 aa, Artificial Sequence
  Synthetic
GFSLTSYG

SEQ ID NO: 161 - protein, 7 aa, Artificial Sequence
  Synthetic
IWGDGNT

SEQ ID NO: 162 - protein, 9 aa, Artificial Sequence
  Synthetic
ANLYVLFAY

SEQ ID NO: 163 - protein, 5 aa, Artificial Sequence
  Synthetic
SSVSY

SEQ ID NO: 164 - protein, 115 aa, Artificial Sequence
  Synthetic
QVQLKESGPGLVAPSQSLSITCTVSGFSLTSYGVSWVRQPPGKGLEWLGVIWGDGNTNYHSALISRLSISKDNSKSQVFLKLNSLQTDDTATYYCANLYVLFAYWGQGTLVTVSA

SEQ ID NO: 165 - skipped

SEQ ID NO: 166 - protein, 106 aa, Artificial Sequence
  Synthetic
ENVLTQSPAIMSASPGEKVTMTCSASSSVSYMHWYQQKSSTSPKLWIYGTSKLASGVPGRFSGSGSGNSYSLTISSMEAEDVATYYCFQGSGYPYTFGGGTKLEIK

SEQ ID NO: 167 - skipped

SEQ ID NO: 168 - protein, 8 aa, Artificial Sequence
  Synthetic
GYTFTNYW

SEQ ID NO: 169 - protein, 8 aa, Artificial Sequence
  Synthetic
INPTNGGT

SEQ ID NO: 170 - protein, 10 aa, Artificial Sequence
  Synthetic
ARSGGYDFDY

SEQ ID NO: 171 - protein, 11 aa, Artificial Sequence
  Synthetic
QSLLDSDGKTY

SEQ ID NO: 172 - protein, 3 aa, Artificial Sequence
  Synthetic
LVS

SEQ ID NO: 173 - protein, 9 aa, Artificial Sequence
  Synthetic
WQGTYFPLT

SEQ ID NO: 174 - protein, 117 aa, Artificial Sequence
  Synthetic
QVQLQQPGTELVKPGASVKLSCKASGYTFTNYWIHWVKQRPGQGLEWIGDINPTNGGTNYNEKFKTKAILTVDRSSSTAYMQVSSLTSEDSAVYYCARSGGYDFDYWGQGTTLTVSS

SEQ ID NO: 175 - skipped

SEQ ID NO: 176 - protein, 112 aa, Artificial Sequence
  Synthetic
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTYFPLTFGAGTKLELK

SEQ ID NO: 177 - protein, 118 aa, Artificial Sequence
  Synthetic
QVQLVQSGAEVKKPGASVKVSCKASGYLFTGYYMNWVRQAPGQGLEWMGEINPNTGDTNNNQKFKARVTMTVDRSISTAYMELSRLRSDDTAVYYCVRRDYGTSLDYWGQGTLVTVSS

SEQ ID NO: 178 - protein, 118 aa, Artificial Sequence
  Synthetic
QVQLVQSGAEVKKPGASVKVSCKASGYLFTGYYMNWVRQAPGQGLEWMGEINPNTGDTNNNQKFKARVTMTVDRSTSTAYMELSSLRSEDTAVYYCVRRDYGTSLDYWGQGTLVTVSS

SEQ ID NO: 179 - protein, 118 aa, Artificial Sequence
  Synthetic
QVQLVQSGAEVKKPGASVKVSCKASGYLFTGYYMNWVRQAPGQRLEWMGEINPNTGDTNNNQKFKARVTITVDRSASTAYMELSSLRSEDTAVYYCVRRDYGTSLDYWGQGTLVTVSS

SEQ ID NO: 180 - protein, 107 aa, Artificial Sequence
  Synthetic
DVQITQSPSSLSASVGDRVTITCRASKSINKYLAWYQQKPGKAPKLLIHSGSTLQSGVPSRFSGSRSGTDFTLTISSLQPEDFATYYCQQHNEYPITFGQGTKLEIK

SEQ ID NO: 181 - protein, 107 aa, Artificial Sequence
  Synthetic
DVQITQSPSFLSASVGDRVTITCRASKSINKYLAWYQQKPGKAPKLLIHSGSTLQSGVPSRFSGSRSGTEFTLTISSLQPEDFATYYCQQHNEYPITFGQGTKLEIK

SEQ ID NO: 182 - protein, 107 aa, Artificial Sequence
  Synthetic
DVQITQSPSSLSASVGDRVTITCRASKSINKYLAWYQQKPGKVPKLLIHSGSTLQSGVPSRFSGSRSGTDFTLTISSLQPEDFATYYCQQHNEYPITFGQGTKLEIK

SEQ ID NO: 183 - protein, 107 aa, Artificial Sequence
  Synthetic
DVQITQSPSTLSASVGDRVTITCRASKSINKYLAWYQQKPGKAPKLLIHSGSTLQSGVPSRFSGSRSGTEFTLTISSLQPDDFATYYCQQHNEYPITFGQGTKLEIK

SEQ ID NO: 184 - protein, 12 aa, Artificial Sequence
  Synthetic
QSLLQSGNQDNY

SEQ ID NO: 185 - protein, 12 aa, Artificial Sequence
  Synthetic
QSLLNAGNQDNY

SEQ ID NO: 186 - protein, 12 aa, Artificial Sequence
  Synthetic
QSLLSSGNQDNY

SEQ ID NO: 187 - protein, 12 aa, Artificial Sequence
  Synthetic
QSLLYSGNQDNY

SEQ ID NO: 188 - protein, 12 aa, Artificial Sequence
  Synthetic
QSLLASGNQDNY

SEQ ID NO: 189 - protein, 120 aa, Artificial Sequence; identical to SEQ ID NO: 109
  Synthetic

SEQ ID NO: 190 - protein, 120 aa, Artificial Sequence
  Synthetic
QVQLVQSGAEVKKPGASVKVSCKASDYTFTSYWMDWVRQAPGQGLEWMGNIYPSDSKTHYNQKFKDRVTMTVDKSTSTAYMELSSLRSEDTAVYYCARRGGYYAYAMDYWGQGTLVTVSS

SEQ ID NO: 191 - protein, 120 aa, Artificial Sequence
  Synthetic
QVQLVQSGAEVKKPGASVKVSCKASDYTFTSYWMDWVRQAPGQRLEWMGNIYPSDSKTHYNQKFKDRVTITVDKSASTAYMELSSLRSEDTAVYYCARRGGYYAYAMDYWGQGTLVTVSS

SEQ ID NO: 192 - protein, 120 aa, Artificial Sequence
  Synthetic
QVQLVQSGAEVKKPGASVKVSCKASDYTFTSYWMDWVRQAPGQGLEWMGNIYPSDSKTHYNQKFKDRVTMTVDKSISTAYMELSRLRSDDTAVYYCARRGGYYAYAMDYWGQGTLVTVSS

SEQ ID NO: 193 - protein, 120 aa, Artificial Sequence
  Synthetic
QVQLVQSGAEVKKPGSSVKVSCKASDYTFTSYWMDWVRQAPGQGLEWMGNIYPSDSKTHYNQKFKDRVTITVDKSTSTAYMELSSLRSEDTAVYYCARRGGYYAYAMDYWGQGTLVTVSS

SEQ ID NO: 194 - protein, 120 aa, Artificial Sequence
  Synthetic
QVQLVQSGAEVKKPGASVKVSCKASDYTFTSYWMDWVRQAPGQGLEWMGNIYPSDSKTHYNQKFKDRVTMTVDKSTSTAYMELRSLRSDDTAVYYCARRGGYYAYAMDYWGQGTLVTVSS

SEQ ID NO: 195 - protein, 113 aa, Artificial Sequence
  Synthetic
DIVMTQSPSSLSVSAGEKVTMRCKSSQSLLQSGNQDNYLAWYQQKPGQPPKLLLYGARIRESGVPDRFIGSGSGTDFTLTISSVQAEDLAVYYCQNDHSYPYTFGGGTKVEIK

SEQ ID NO: 196 - protein, 113 aa, Artificial Sequence
  Synthetic
DIVMTQSPSSLSVSAGEKVTMRCKSSQSLLNAGNQDNYLAWYQQKPGQPPKLLLYGARIRESGVPDRFIGSGSGTDFTLTISSVQAEDLAVYYCQNDHSYPYTFGGGTKVEIK

SEQ ID NO: 197 - protein, 113 aa, Artificial Sequence
  Synthetic
DIVMTQSPSSLSVSAGEKVTMRCKSSQSLLSSGNQDNYLAWYQQKPGQPPKLLLYGARIRESGVPDRFIGSGSGTDFTLTISSVQAEDLAVYYCQNDHSYPYTFGGGTKVEIK

SEQ ID NO: 198 - protein, 113 aa, Artificial Sequence
  Synthetic
DIVMTQSPSSLSVSAGEKVTMRCKSSQSLLYSGNQDNYLAWYQQKPGQPPKLLLYGARIRESGVPDRFIGSGSGTDFTLTISSVQAEDLAVYYCQNDHSYPYTFGGGTKVEIK

SEQ ID NO: 199 - protein, 113 aa, Artificial Sequence
  Synthetic
DIVMTQSPSSLSVSAGEKVTMRCKSSQSLLASGNQDNYLAWYQQKPGQPPKLLLYGARIRESGVPDRFIGSGSGTDFTLTISSVQAEDLAVYYCQNDHSYPYTFGGGTKVEIK

SEQ ID NO: 200 - protein, 113 aa, Artificial Sequence
  Synthetic
DIVMTQSPDSLAVSLGERATINCKSSQSLLNSGNQDNYLAWYQQKPGQPPKLLIYGARIRESGVPDRFSGSGSGTDFTLTISSLQAEDLAVYYCQNDHSYPYTFGGGTKVEIK

SEQ ID NO: 201 - protein, 113 aa, Artificial Sequence
  Synthetic
DIVMTQSPLSLPVTPGEPASISCKSSQSLLNSGNQDNYLAWYLQKPGQSPQLLIYGARIRESGVPDRFSGSGSGTDFTLKISRVEAEDLGVYYCQNDHSYPYTFGGGTKVEIK

SEQ ID NO: 202 - protein, 113 aa, Artificial Sequence
  Synthetic
EIVMTQSPATLSVSPGERATLSCKSSQSLLNSGNQDNYLAWYQQKPGQAPRLLIYGARIRESGIPARFSGSGSGTEFTLTISSLQSEDLAVYYCQNDHSYPYTFGGGTKVEIK

SEQ ID NO: 203 - protein, 113 aa, Artificial Sequence
  Synthetic
DIQMTQSPSSLSASVGDRVTITCKSSQSLLNSGNQDNYLAWYQQKPGKAPKLLIYGARIRESGVPSRFSGSGSGTDFTLTISSLQPEDLATYYCQNDHSYPYTFGGGTKVEIK

SEQ ID NO: 204 - DNA, 360 bp, Artificial Sequence
  Synthetic
caggtgcagctgctgcagcccggcgctgagctggtgagacccggaagcagcgtgaagctgagctgcaaagccagcgactacacattcacaagctactggatggactgggtgaaacagagacctggccagggactggaatggattggaaacatctaccccagtgacagcaagacccactacaaccagaagttcaaggacaaagccaccctgaccgtggacaagagcagcagcaccgcctacatgcagctgaacagcctgaccagcgaagacagcgccgtgtactactgcgccagaagaggcggctactacgcctatgccatggactactgggggcagggaaccagcgtgaccgtgagcagc

SEQ ID NO: 205 - DNA, 339 bp, Artificial Sequence
  Synthetic
gacattgtgatgacccagagccccagcagcctgagcgtgagcgccggagaaaaagtgaccatgagatgcaagagcagccagagcctgctgaacagcgggaaccaggacaactacctggcctggtatcaacagaagcccggccagccccccaaactgctgctgtatggagccagaatcagagagagcggcgtgcccgacagattcatcggcagcggcagcggaaccgacttcaccctgaccatcagcagcgtgcaggctgaggacctggccgtgtactactgccagaacgaccacagctacccctatacctttggaggcggcaccaaggtggagatcaaa

SEQ ID NO: 206 - DNA, 369 bp, Artificial Sequence
  Synthetic
caggtccagctgcagcagtctggggctgaactggcaagacctggtgcctcagtgaagatgtcctgcaaggcttctggctacacctttactagcaacacgatgcactgggtaaaacagaggcctggacagggtctggaatggattggatacattaatcctggcagtgcttatactaactacaatcagaagttcaatgacaaggccacattgactgcagacaagtcctccagcacagcctccatgcaactgagcagcctgacatctgaggactctgcagtctattactgtgcaagactcgcgacctactatgataacgacggatatgctatggactattggggtcaaggaacctcagtcaccgtctcctca

SEQ ID NO: 207 - DNA, 321 bp, Artificial Sequence
  Synthetic
gacatccagatgacacagtctacatcctccctgtctgcctctctgggagacagagtcaccatcagttgcagggcaagtcaggacattagtaattatttaaactggtatcagcagaaaccagatggaactgttaaactcctgatctactacacatcaaaattacactcaggagtcccatcaaggttcagtggcagtgggtctggaacagatttttctctcaccattaccaacctggagcaagaagattttgccacttacttttgccaacagggtcatacgcttccgtggacgttcggtggaggcaccaaactggagatcaaa

SEQ ID NO: 208 - DNA, 321 bp, Artificial Sequence
  Synthetic
gatatccagatgacacagactacatcctccctgtctgcctctctgggagacagagtcaccatcagttgcagggcaagtcaggacattagtaattatttaaactggtatcagcagaaaccagatggaactgttaaactcctgatctactacacatcaaaattacactcaggagtcccatcaaggttcagtggcagtgggtctggaacagatttttctctcaccattaccaacctggagcaagaagattttgccacttacttttgccaacagggtcatacgcttccgtggacgttcggtggaggcaccaaactggagatcaaa

SEQ ID NO: 209 - DNA, 372 bp, Artificial Sequence
  Synthetic
gaggtcctgctgcaacagtctggacctgaactggtgaagcctggggcttcagtgaagataccctgcaaggcttctggatacggattcactgactacaacatggactggataaaacagagccatggaaagagccttgagtggattggagatattaatcctcacaatggtgatactatctacaaccagaagttcaagggcaaggccaaattgactgtagacaagtcctccaccactgcctacatggaactccgcagcctggcatctgaggacaccgcagtctattactgtgcaaggaggggggcatattactacggtagtagccactatgctctggacttctggggtcaaggaacctcagtcaccgtctcctca

SEQ ID NO: 210 - DNA, 321 bp, Artificial Sequence
  Synthetic
gatatccagatgacacagactacatcctccctgtctgcctctctgggagacagagtcaccatcagttgcagggcaagtcaggacattaccaattatttaaactggtatcagcagaaaccagatggaactgttaaactcctgatctactacacatcaagattattctcaggagtcccatcaaggttcagtggcagtgggtctggaacagattattctctcaccattagcaacctgaaacgagaagatattgccacttacttttgccaacagggtaatacgcttccgtggacgttcggtggaggcaccaagctggaaatcaaa

SEQ ID NO: 211 - DNA, 354 bp, Artificial Sequence
  Synthetic
gaggttcagctgcagcagtctggacctgagctggtgaagcctggggcttcagtgaagatatcctgcaaggcctctggttacttattcactggctactacatgaactgggtgaaacaaagtcctgaaaagagtcttgagtggattggagagattaatcctaacaaaggtgatagtaatctcaaccagaatttcaaggccaaggccacattgactgttgacaaatcctccagcacagcctacatgcagttcaagagcctgacatttgaggactctgcagtctatttctgtgtaagaagggactacggcactagtcttgaccactggggccaaggcacgactctcacagtctcctca

SEQ ID NO: 212 - DNA, 321 bp, Artificial Sequence
  Synthetic
gatgtccagataacccagtctccatcttatcttgctacatctcctggagaaaccattactattaattgcagggcaagtaagagtattagcaaatatttagcctggtatcaagagaaacctgggaaaactaagaagcttcttatctactctggatccactttgcatactgcaattccatcaaggttcagtggccgtggatttggtacagatttcactctcaccatcagtagcctggagcctgaagattttgcaatgtattactgtcaacagcataatgaatacccaatcacattcggtggtgggaccaagctggagctgaaa

SEQ ID NO: 213 - DNA, 354 bp, Artificial Sequence
  Synthetic
gaggtccagctgcagcagtctggacctgagttggtgaagcctggggcttcagtgaagatatcctgcaaggctactggttacttattcactggctactacatgaactgggtgaagcaaagtcctgaaaagagccttgagtggattggagagatcaatcctaacactggtgatactaacaacaaccagaagttcaaggccaaggccacaatgactgttgacagatcctccagtacagcctacatgcagctcaagagcctgacatctgaggactctgcagtctattattgtgtaagaagggactacggtactagccttgactactggggccaaggcaccactctcacagtctcctca

SEQ ID NO: 214 - DNA, 321 bp, Artificial Sequence
  Synthetic
gatgtccagataacccagtctccatcttatcttgctgcatctcctggagaaaccattactattaattgcagggcaagtaagagtattaacaaatatttagcctggtatcaagagaaacctgggaaaactaataagcttcttatccactctggatccactttgcactctggaattccatcaaggttcagtggcagtagatctggaacagatttcactctcaccatcagtaacctggagcctgaagattttgcaatgtattactgtcaacaacataatgaatacccgctcacgttcggtgctgggaccaagctggagctgaaa

SEQ ID NO: 215 - DNA, 369 bp, Artificial Sequence
  Synthetic
caggtgcagctgcagcagagcggcgccgagctggctagacccggagcttccgtgaagatgagctgcaaagccagcggctacacattcacatccaacacaatgcactgggtgaaacagaggcctggccagggactggagtggattgggtacatcaaccccggcagcgcctacaccaactacaaccagaaattcaatgacaaagccacactgaccgccgacaaaagcagcagcacagccagcatgcagctgagcagcctgacctccgaggacagcgctgtgtactactgcgccagactggccacctactacgacaacgacggatacgccatggattactggggacagggaaccagcgtgaccgtgagcagc

SEQ ID NO: 216 - DNA, 318 bp, Artificial Sequence
  Synthetic
gagaatgtgctgacccagagccccgccatcatgagtgccagcccaggagagaaggtgacaatgacatgcagcgccagcagcagcgtgaattacatgtactggtaccagcagagaagcagcaccagccccaagctgtggatctacggcaccagcaagctggccagcggagtgcccggcagattctccggaagtggaagcggaaatagctacagcctgactatcagcagcatggaggccgaagatgttgctacctactactgctttcagggcagcggctacccctacaccttcggcggcggcaccaagctggagatcaag

SEQ ID NO: 217 - DNA, 345 bp, Artificial Sequence
  Synthetic
caggtgcagctgaaggaaagcggccccggcctggtggccccatctcagagtctgagcatcacctgcacagtgagcggcttcagcctgacaagctacggcgtgagctgggtgagacagccccccggaaagggcctggaatggctgggcgtgatttggggcgacggcaacacaaactaccacagcgccctgatcagcagactgagcatcagcaaggacaatagcaagagccaggtgttcctgaagctgaacagcctgcagaccgacgacaccgccacctactactgcgccaacctgtacgtgctgttcgcctactggggccagggcaccctggtgaccgtgagcgct

SEQ ID NO: 218 - DNA, 318 bp, Artificial Sequence
  Synthetic
gagaatgtgctgacccagagccccgccatcatgagtgccagcccaggagagaaggtgacaatgacatgcagcgccagcagcagcgtgagttacatgcactggtaccagcagaagagcagcaccagccccaaactgtggatctacgggaccagcaagctggccagcggagtgcccggaagattcagtggcagcggaagcggcaatagctacagcctgactatcagcagcatggaggccgaagatgttgctacctactactgttttcagggaagcggctacccatacaccttcgggggggggaccaagctggaaatcaag

SEQ ID NO: 219 - DNA, 351 bp, Artificial Sequence
  Synthetic
caggtgcagctgcagcagccaggcacagagctggtgaagcccggcgccagcgtgaaactgagctgcaaagccagcggctacaccttcacaaactactggattcattgggtgaagcagaggcctggccagggactggagtggatcggcgacattaaccccacaaacggaggcacaaactacaacgagaaattcaagaccaaagccatcctgaccgtggacagaagcagtagcacagcctacatgcaggtgagcagcctgaccagcgaggacagcgccgtgtactattgcgccagaagcggcggctacgacttcgactactggggccagggcaccaccctgaccgtgagcagc

SEQ ID NO: 220 - DNA, 336 bp, Artificial Sequence
  Synthetic
gacgtggtgatgacccagacccccctgacactgagcgtgaccatcggacagcccgccagcatcagctgcaagagcagccagagcctgctggacagcgacggaaaaacatatctgaattggctgctgcagaggcctggccagagccccaaaagactgatctacctggtgagcaagctggacagcggggtgcccgacagattcaccggcagcggaagcggcaccgacttcaccctgaagatctccagagtggaagccgaggacctgggcgtgtactactgctggcagggaacctacttccccctgacctttggagccggcaccaagctggaactgaag

SEQ ID NO: 221 - protein, 107 aa, Artificial Sequence
  Synthetic
DIQMTQTTSSLSASLGDRVTISCRASQDISNYLNWYQQKPDGTVKLLIYYTSKLHSGVPSRFSGSGSGTDFSLTITNLEQEDFATYFCQQGHTLPWTFGGGTKLEIK

SEQ ID NO: 222 - protein, 5 aa, Artificial Sequence
  Synthetic
SYWIT

SEQ ID NO: 223 - protein, 17 aa, Artificial Sequence
  Synthetic
DISPAGGGRNYNERFKN

SEQ ID NO: 224 - protein, 9 aa, Artificial Sequence
  Synthetic
GDSTVDLDY

SEQ ID NO: 225 - protein, 16 aa, Artificial Sequence
  Synthetic
RSSKSLLHSNGNTYLY

SEQ ID NO: 226 - protein, 7 aa, Artificial Sequence
  Synthetic
RVSNLAS

SEQ ID NO: 227 - protein, 8 aa, Artificial Sequence
  Synthetic
MQHLEYPY

SEQ ID NO: 228 - DNA, 354 bp, Artificial Sequence
  Synthetic
gaggttcagctgcagcagtctgggactggacttgtgaagcctgggacttcagtgaagttgtcctgcaaggcttctggctacagcttcaccagctactggataacctgggtgaaacagaggcctggacaaggccttgagtggattggagatatttctcctgctggtggtggtcgtaactacaatgagagattcaagaacaaggccacactgactgtagacgcatcctccagcacagcctacatgcagctcagcagcctgacatctgaggactctgcggtctattactgtgtaagaggtgatagtacggtagacttagactactggggccaaggcaccactctcacagtctcctca

SEQ ID NO: 229 - DNA, 336 bp, Artificial Sequence
  Synthetic
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcgggtgtccaaccttgcctcaggagtcccagacaggttcagtggcagtgggtcagggactgctttcacactgagaatcagtagagtggaggctgaggatgtgggtgtttattactgtatgcaacatctagaatatccgtacacgttcggaggggggaccaagctggaaataaaa

(19) United States
(12) Patent Application Publication
(10) Pub. No.: US 2024/0052052 A1
(43) Pub. Date: Feb. 15, 2024

(54) NOVEL ANTI-CD24 ANTIBODIES

(71) Applicant: ANTENGENE BIOLOGICS LIMITED, Shanghai (CN)

(72) Inventors: Peng CHEN, Shanghai (CN); Bing HOU, Shanghai (CN); Min DENG, Shanghai (CN); Yun LIU, Shanghai (CN); Jiamei LUO, Shanghai (CN); Hui YUWEN, Shanghai (CN); Bo SHAN, Shanghai (CN); Jay MEI, Shanghai (CN)

(21) Appl. No.: 18/264,436

(22) PCT Filed: Feb. 8, 2022
(86) PCT No.: PCT/US2022/015711

§ 371 Date: Aug. 7, 2023

(30) Foreign Application Priority Data
Feb. 8, 2021 (WO) PCT/CN2021/076075
Jan. 25, 2022 (CN) 202210088172.3

(51) Int. Cl. C07K 16/28; C07K 14/725; G01N 33/574

(52) U.S. Cl. CPC C07K 16/2896; C07K 16/2803; C07K 14/7051; G01N 33/57492; C07K 2317/31; C07K 2317/622; C07K 2319/03; C07K 2319/02; G01N 2333/70596

(57) ABSTRACT

The present disclosure provides anti-CD24 antibody, and anti-CD24/CD47 bispecific antibody molecules, isolated polynucleotides encoding the same, pharmaceutical compositions comprising the same, and the uses thereof

### FIELD OF THE INVENTION

The present disclosure generally relates to novel anti-CD24 antibodies.

### BACKGROUND

Cluster of differentiation 24 (CD24, UniProt: P25063), also known as Heat Stable Atigen (HSA) or Small Cell Lung Carcinoma Cluster 4 Antigen, is a 32-amino acid mucin-like glycosylphosphatidylinositol (GPI)-anchored molecule, which is heavily glycosylated and anchored via a GPI link to the cell surface. CD24 can interact with Sialic Acid Binding Ig Like Lectin 10 (Siglec-10) expressed on innate immune cells (e.g., macrophages) so as to diminish damaging inflammatory responses to infection, sepsis, liver damage, and chronic graft versus host disease (Pirruccello el al., J. Immunol. 136, 3779-3784 (1986); Chen et al., Glycobiology 9, 800-806 (2014); Chen et al., Cell 152(3), 467-478; Chen G Y et al. Nature Biotechnology 29, 428-435 (2011); Chen G Y et al. Science 323 (5922), 1722-1725 (2009); and Toubai T et al. Blood 123(22), 3512-3513 (2014)). Binding of CD24 to Siglec-10 induces an inhibitory signaling cascade mediated by SHP-1 and/or SHP-2 phosphatases associated with the two immunoreceptor tyrosine-based inhibition motifs (ITIMs) in the cytoplasmic tail of Siglec-10, thereby blocking toll-like receptor (TLR)-mediated inflammation and the cytoskeletal rearrangement required for cellular engulfment by macrophages (Crocker P R et al., Nature Reviews Immunology. 7, 255-266 (2007); Abram C L et al., J. Leuko.c Biol. 102(3), 657-675 (2017) and Dietrich J et al., J. Immunol. 166(4). 2514-2521 (2001) 12-14).

Overexpression of CD24 has been observed in various cancer cells based on immunohistochemical studies, including without limitation, breast cancer (85%), rectal cancer (84%), ovarian cancer (83%), pancreatic cancer (72%), bladder cancer (62%), cholangiocarcinoma (51%), prostatic cancer (48%), and small cell lung cancer (45%). A meta-analysis of 28 studies revealed that CD24 is overexpressed in 68% of human cancers, and CD24 expression was correlated with a higher self-renewal ability, more metastases, and a poor prognosis (Krisiansen G. et al., Am J Pathol, 161:1215-1221, 2002; Krisiansen G. et al., Clin cancer Res, 9:4906-4913, 2003; Krisiansen G. et al., Br J Cancer, 88:231-236, 2003; Kristiansen G. et al., Prostate, 58:182-192, 2004; Hocob J. et al., Pancreatology, 4:454-460, 2004; Samuel E D, BioMed Central, 3:3-15, 2004; Min-Cheng S. et al., Cancer letter, 1-6, 2005 and Lee et al., Oncol. Rep. 2009, 22, 1149-1156). A recent study showed that the CD24-mediated inhibition of the innate immune system can be exploited by cancer cells as a mechanism to avoid clearance by macrophages expressing Siglec-10 (Amira et al., Nature. 2019 August; 572(7769): 392-396).

Despite of the development of therapeutics targeting the CD24, there is a significant need for novel anti-CD24 antibodies.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel anti-CD24 antibody molecules, amino acid and nucleotide sequences thereof, and uses thereof.

In one aspect, the present disclosure provides an isolated antibody or an antigen-binding fragment thereof, comprising 1, 2, or 3 heavy chain complementarity determining region (CDR) sequences selected from the group consisting of: SEQ ID NOs: 1, 3, 5, 7, 9, 11, 14, 16, 17, 18, 19, 21, 23, 25, 27, 28, 30, 32, 34, 36, 38, 40, 42, 44, 46, 48, 95, 96, 97, 104, 105, 106, 113, 114, 115, 126, 127, 128, 136, 137, 150, 151, 152, 160, 161, 162, 168, 169 170, 222, 223 and 224 and/or 1, 2, or 3 light chain CDR sequences selected from the group consisting of: SEQ ID NOs: 2, 4, 6, 8, 10, 12, 13, 15, 20, 22, 24, 26, 29, 31, 33, 35, 37, 39, 41, 43, 45, 47, 49, 98, 99, 100, 107, 108, 116, 117, 129, 130, 131, 138, 139, 140, 145, 153, 154, 155, 163, 171, 172, 173, 184, 185, 186, 187, 188, 225, 226 and 227.

In some embodiments, the antibody or antigen-binding fragment thereof provided herein comprises a heavy chain variable region selected from the group consisting of:
a) a heavy chain variable region comprising 1, 2, or 3 CDR sequences selected from SEQ ID NO: 1, SEQ ID NO: 3, and SEQ ID NO: 5;
b) a heavy chain variable region comprising 1, 2, or 3 CDR sequences selected from SEQ ID NO: 7, SEQ ID NO: 9, and SEQ ID NO: 11;
c) a heavy chain variable region comprising 1, 2, or 3 CDR sequences selected from SEQ ID NO: 7, SEQ ID NO: 14, and SEQ ID NO: 11;
d) a heavy chain variable region comprising 1, 2, or 3 CDR sequences selected from SEQ ID NO: 16, SEQ ID NO: 17, and SEQ ID NO: 18;
e) a heavy chain variable region comprising 1, 2, or 3 CDR sequences selected from SEQ ID NO: 19, SEQ ID NO: 21, and SEQ ID NO: 23,
f) a heavy chain variable region comprising 1, 2, or 3 CDR sequences selected from SEQ ID NO:25, SEQ ID NO: 27, and SEQ ID NO: 28;
g) a heavy chain variable region comprising 1, 2, or 3 CDR sequences selected from SEQ ID NO:25, SEQ ID NO: 30, and SEQ ID NO: 28;
h) a heavy chain variable region comprising 1, 2, or 3 CDR sequences selected from SEQ ID NO: 32, SEQ ID NO: 34, and SEQ ID NO: 36;
i) a heavy chain variable region comprising 1, 2, or 3 CDR sequences selected from SEQ ID NO: 38, SEQ ID NO: 40, and SEQ ID NO: 42;
j) a heavy chain variable region comprising 1, 2, or 3 CDR sequences selected from SEQ ID NO: 44, SEQ ID NO: 46, and SEQ ID NO: 48;
k) a heavy chain variable region comprising 1, 2, or 3 CDR sequences selected from SEQ ID NO: 95, SEQ ID NO: 96, and SEQ ID NO: 97;
l) a heavy chain variable region comprising 1, 2, or 3 CDR sequences selected from SEQ ID NO: 104, SEQ ID NO: 105, and SEQ ID NO: 106;
m) a heavy chain variable region comprising 1, 2, or 3 CDR sequences selected from SEQ ID NO: 113, SEQ ID NO: 114, and SEQ ID NO: 115;
n) a heavy chain variable region comprising 1, 2, or 3 CDR sequences selected from SEQ ID NO: 126, SEQ ID NO: 127, and SEQ ID NO: 128;
o) a heavy chain variable region comprising 1, 2, or 3 CDR sequences selected from SEQ ID NO: 25, SEQ ID NO: 136, and SEQ ID NO: 137;
p) a heavy chain variable region comprising 1, 2, or 3 CDR sequences selected from SEQ ID NO: 150, SEQ ID NO: 151, and SEQ ID NO: 152;
q) a heavy chain variable region comprising 1, 2, or 3 CDR sequences selected from SEQ ID NO: 160, SEQ ID NO: 161, and SEQ ID NO: 162;
r) a heavy chain variable region comprising 1, 2, or 3 CDR sequences selected from SEQ ID NO: 168, SEQ ID NO: 169, and SEQ ID NO: 170, and
s) a heavy chain variable region comprising 1, 2, or 3 CDR sequences selected from SEQ ID NO: 222, SEQ ID NO: 223, and SEQ ID NO: 224.

In some embodiments, the antibody or antigen-binding fragment thereof provided herein comprises a light chain variable region selected from the group consisting of:
a) a light chain variable region comprising 1, 2, or 3 CDR sequences selected from SEQ ID NO: 2, SEQ ID NO: 4, and SEQ ID NO: 6;
b) a light chain variable region comprising 1, 2, or 3 CDR sequences selected from SEQ ID NO: 8, SEQ ID NO: 10, and SEQ ID NO: 12;
c) a light chain variable region comprising 1, 2, or 3 CDR sequences selected from SEQ ID NO: 13, SEQ ID NO: 10 and SEQ ID NO: 15;
d) a light chain variable region comprising 1, 2, or 3 CDR sequences selected from SEQ ID NO: 20, SEQ ID NO: 22, and SEQ ID NO: 24;
e) a light chain variable region comprising 1, 2, or 3 CDR sequences selected from SEQ ID NO: 26, SEQ ID NO: 22, and SEQ ID NO:24;
f) a light chain variable region comprising 1, 2, or 3 CDR sequences selected from SEQ ID NO: 29, SEQ ID NO: 22, and SEQ ID NO: 31;
g) a light chain variable region comprising 1, 2, or 3 CDR sequences selected from SEQ ID NO: 33, SEQ ID NO: 35, and SEQ ID NO: 37;
h) a light chain variable region comprising 1, 2, or 3 CDR sequences selected from SEQ ID NO: 39, SEQ ID NO: 41, and SEQ ID NO: 43;
i) a light chain variable region comprising 1, 2, or 3 CDR sequences selected from SEQ ID NO: 45, SEQ ID NO: 47, and SEQ ID NO: 49;
j) a light chain variable region comprising 1, 2, or 3 CDR sequences selected from SEQ ID NO: 98, SEQ ID NO: 99, and SEQ ID NO: 100.
k) a light chain variable region comprising 1, 2, or 3 CDR sequences selected from SEQ ID NO: 107, SEQ ID NO: 108, and SEQ ID NO: 37;
l) a light chain variable region comprising 1, 2, or 3 CDR sequences selected from SEQ ID NO: 13, SEQ ID NO: 116, and SEQ ID NO: 117;
m) a light chain variable region comprising 1, 2, or 3 CDR sequences selected from SEQ ID NO: 129, SEQ ID NO: 130, and SEQ ID NO: 131;
n) a light chain variable region comprising 1, 2, or 3 CDR sequences selected from SEQ ID NO: 138, SEQ ID NO: 139, and SEQ ID NO: 140;
o) a light chain variable region comprising 1, 2, or 3 CDR sequences selected from SEQ ID NO: 29, SEQ ID NO: 145, and SEQ ID NO: 24;
p) a light chain variable region comprising 1, 2, or 3 CDR sequences selected from SEQ ID NO: 153, SEQ ID NO: 154, and SEQ ID NO: 155;
q) a light chain variable region comprising 1, 2, or 3 CDR sequences selected from SEQ ID NO: 163, SEQ ID NO: 154, and SEQ ID NO: 155;
r) a light chain variable region comprising 1, 2, or 3 CDR sequences selected from SEQ ID NO: 171, SEQ ID NO: 172, and SEQ ID NO: 173;
s) a light chain variable region comprising 1, 2, or 3 CDR sequences selected from SEQ ID NO: 184, SEQ ID NO: 108, and SEQ ID NO: 37;
t) a light chain variable region comprising 1, 2, or 3 CDR sequences selected from SEQ ID NO: 185, SEQ ID NO: 108, and SEQ ID NO: 37;
u) a light chain variable region comprising 1, 2, or 3 CDR sequences selected from SEQ ID NO: 186, SEQ ID NO: 108, and SEQ ID NO: 37;
v) a light chain variable region comprising 1, 2, or 3 CDR sequences selected from SEQ ID NO: 187, SEQ ID NO: 108, and SEQ ID NO: 37;
w) a light chain variable region comprising 1, 2, or 3 CDR sequences selected from SEQ ID NO: 188, SEQ ID NO: 108, and SEQ ID NO: 37; and
x) a light chain variable region comprising 1, 2, or 3 CDR sequences selected from SEQ ID NO: 225, SEQ ID NO: 226, and SEQ ID NO: 227.

In some embodiments, the antibody or antigen-binding fragment thereof provided herein comprises:
a) a heavy chain variable region comprising 1, 2, or 3 CDR sequences selected from SEQ ID NO: 1, SEQ ID NO: 3, and SEQ ID NO: 5; and a light chain variable region comprising 1, 2, or 3 CDR sequences selected from SEQ ID NO: 2, SEQ ID NO: 4, and SEQ ID NO: 6;
b) a heavy chain variable region comprising 1, 2, or 3 CDR sequences selected from SEQ ID NO: 7, SEQ ID NO: 9, and SEQ ID NO: 11; and a light chain variable region comprising 1, 2, or 3 CDR sequences selected from SEQ ID NO: 8, SEQ ID NO: 10, and SEQ ID NO: 12;
c) a heavy chain variable region comprising 1, 2, or 3 CDR sequences selected from SEQ ID NO: 7, SEQ ID NO: 14, and SEQ ID NO: 11; and a light chain variable region comprising 1, 2, or 3 CDR sequences selected from SEQ ID NO: 13, SEQ ID NO: 10 and SEQ ID NO: 15;
d) a heavy chain variable region comprising 1, 2, or 3 CDR sequences selected from SEQ ID NO: 16, SEQ ID NO: 17, and SEQ ID NO: 18; and a light chain variable region comprising 1, 2, or 3 CDR sequences selected from SEQ ID NO: 13, SEQ ID NO: 10, and SEQ ID NO: 15;
e) a heavy chain variable region comprising 1, 2, or 3 CDR sequences selected from SEQ ID NO: 19, SEQ ID NO: 21, and SEQ ID NO: 23; and a light chain variable region comprising 1, 2, or 3 CDR sequences selected from SEQ ID NO: 20, SEQ ID NO: 22, and SEQ ID NO: 24;
f) a heavy chain variable region comprising 1, 2, or 3 CDR sequences selected from SEQ ID NO:25, SEQ ID NO: 27, and SEQ ID NO: 28; and a light chain variable region comprising 1, 2, or 3 CDR sequences selected from SEQ ID NO: 26, SEQ ID NO: 22, and SEQ ID NO:24;
g) a heavy chain variable region comprising 1, 2, or 3 CDR sequences selected from SEQ ID NO:25, SEQ ID NO: 30, and SEQ ID NO: 28; and a light chain variable region comprising 1, 2, or 3 CDR sequences selected from SEQ ID NO: 29, SEQ ID NO: 22, and SEQ ID NO: 31;
h) a heavy chain variable region comprising 1, 2, or 3 CDR sequences selected from SEQ ID NO: 32, SEQ ID NO: 34, and SEQ ID NO: 36; and a light chain variable region comprising 1, 2, or 3 CDR sequences selected from SEQ ID SEQ ID NO: 33, SEQ ID NO: 35, and SEQ ID NO: 37;
i) a heavy chain variable region comprising 1, 2, or 3 CDR sequences selected from SEQ ID NO: 38, SEQ ID NO: 40, and SEQ ID NO: 42; and a kappa light chain variable region comprising 1, 2, or 3 CDR sequences selected from SEQ ID NO: 39, SEQ ID NO: 41, and SEQ ID NO: 43;
j) a heavy chain variable region comprising 1, 2, or 3 CDR sequences selected from SEQ ID NO: 44, SEQ ID NO: 46, and SEQ ID NO: 48; and a kappa light chain variable region comprising 1, 2, or 3 CDR sequences selected from SEQ ID NO: 45, SEQ ID NO: 47, and SEQ ID NO: 49;
k) a heavy chain variable region comprising 1, 2, or 3 CDR sequences selected from SEQ ID NO: 95, SEQ ID NO: 96, and SEQ ID NO: 97; and a kappa light chain variable region comprising 1, 2, or 3 CDR sequences selected from SEQ ID NO: 98, SEQ ID NO: 99, and SEQ ID NO: 100;
l) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 107, SEQ ID NO: 108, and SEQ ID NO: 37;
m) a heavy chain variable region comprising 1, 2, or 3 CDR sequences selected from SEQ ID NO: 113, SEQ ID NO: 114, and SEQ ID NO: 115; and a kappa light chain variable region comprising 1, 2, or 3 CDR sequences selected from SEQ ID NO: 13, SEQ ID NO: 116, and SEQ ID NO: 117;
n) a heavy chain variable region comprising 1, 2, or 3 CDR sequences selected from SEQ ID NO: 126, SEQ ID NO: 127, and SEQ ID NO: 128; and a kappa light chain variable region comprising 1, 2, or 3 CDR sequences selected from SEQ ID NO: 129, SEQ ID NO: 130, and SEQ ID NO: 131;
o) a heavy chain variable region comprising 1, 2, or 3 CDR sequences selected from SEQ ID NO: 25, SEQ ID NO: 136, and SEQ ID NO: 137; and a kappa light chain variable region comprising 1, 2, or 3 CDR sequences selected from SEQ ID NO: 138, SEQ ID NO: 139, and SEQ ID NO: 140;
p) a heavy chain variable region comprising 1, 2, or 3 CDR sequences selected from SEQ ID NO: 25, SEQ ID NO: 30, and SEQ ID NO: 28; and a kappa light chain variable region comprising 1, 2, or 3 CDR sequences selected from SEQ ID NO: 29, SEQ ID NO: 145, and SEQ ID NO: 24;
q) a heavy chain variable region comprising 1, 2, or 3 CDR sequences selected from SEQ ID NO: 150, SEQ ID NO: 151, and SEQ ID NO: 152; and a kappa light chain variable region comprising 1, 2, or 3 CDR sequences selected from SEQ ID NO: 153, SEQ ID NO: 154, and SEQ ID NO: 155;
r) a heavy chain variable region comprising 1, 2, or 3 CDR sequences selected from SEQ ID NO: 160, SEQ ID NO: 161, and SEQ ID NO: 162; and a kappa light chain variable region comprising 1, 2, or 3 CDR sequences selected from SEQ ID NO: 163, SEQ ID NO: 154, and SEQ ID NO: 155;
s) a heavy chain variable region comprising 1, 2, or 3 CDR sequences selected from SEQ ID NO: 168, SEQ ID NO: 169, and SEQ ID NO: 170; and a kappa light chain variable region comprising 1, 2, or 3 CDR sequences selected from SEQ ID NO: 171, SEQ ID NO: 172, and SEQ ID NO: 173;
t) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 184, SEQ ID NO: 108, and SEQ ID NO: 37;
u) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 185, SEQ ID NO: 108, and SEQ ID NO: 37;
v) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 186, SEQ ID NO: 108, and SEQ ID NO: 37;
w) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 187, SEQ ID NO: 108, and SEQ ID NO: 37;
x) a heavy chain variable region comprising 1, 2, or 3 CDR sequences selected from SEQ ID NO: 104, SEQ ID NO: 105, and SEQ ID NO: 106; and a kappa light chain variable region comprising 1, 2, or 3 CDR sequences selected from SEQ ID NO: 188, SEQ ID NO: 108, and SEQ ID NO: 37; or
y) a heavy chain variable region comprising 1, 2, or 3 CDR sequences selected from SEQ ID NO: 222, SEQ ID NO: 223, and SEQ ID NO: 224; and a kappa light chain variable region comprising 1, 2, or 3 CDR sequences selected from SEQ ID NO: 225, SEQ ID NO: 226, and SEQ ID NO: 227.

In some embodiments, the antibody or antigen-binding fragment thereof provided herein comprises a heavy chain variable region selected from the group consisting of: SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 101, SEQ ID NO: 109, SEQ ID NO: 118, SEQ ID NO: 122, SEQ ID NO: 132, SEQ ID NO: 141, SEQ ID NO: 146, SEQ ID NO: 156, SEQ ID NO: 164, SEQ ID NO: 174, SEQ ID NO: 177, SEQ ID NO: 178, SEQ ID NO: 179, SEQ ID NO: 189, SEQ ID NO: 190, SEQ ID NO: 191, SEQ ID NO: 192, SEQ ID NO: 193, SEQ ID NO: 194 and the homologue sequences of at least 80% (e.g., at least 85%, 88%, 90%, 91%, 92%, 93%, 94%, 95%, 96%, 97%, 98%, 99%) sequence identity thereof.

In some embodiments, the antibody or antigen-binding fragment thereof provided herein comprises a light chain variable region selected from the group consisting of: SEQ ID NO: 52, SEQ ID NO: 56, SEQ ID NO: 64, SEQ ID NO: 68, SEQ ID NO: 72, SEQ ID NO: 76, SEQ ID NO: 80, SEQ ID NO: 84, SEQ ID NO: 88, SEQ ID NO: 103, SEQ ID NO: 111, SEQ ID NO: 120, SEQ ID NO: 124, SEQ ID NO: 134, SEQ ID NO: 143, SEQ ID NO: 148, SEQ ID NO: 158, SEQ ID NO: 166, SEQ ID NO: 176, SEQ ID NO: 180, SEQ ID NO: 181, SEQ ID NO: 182, SEQ ID NO: 183, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 221 and the homologue sequences of at least 80% (e.g., at least 85%, 88%, 90%, 91%, 92%, 93%, 94%, 95%, 96%, 97%, 98%, 99%) sequence identity thereof.

In some embodiments, the antibody or antigen-binding fragment thereof provided herein comprises:
a) a heavy chain variable region comprising SEQ ID NO: 50 and a light chain variable region comprising SEQ ID NO: 52;
b) a heavy chain variable region comprising SEQ ID NO: 54 and a light chain variable region comprising SEQ ID NO: 56;
c) a heavy chain variable region comprising SEQ ID NO: 58 and a light chain variable region comprising SEQ ID NO: 64;
d) a heavy chain variable region comprising SEQ ID NO: 62 and a light chain variable region comprising SEQ ID NO: 64;
e) a heavy chain variable region comprising SEQ ID NO: 66 and a light chain variable region comprising SEQ ID NO: 68;
f) a heavy chain variable region comprising SEQ ID NO: 70 and a light chain variable region comprising SEQ ID NO: 72;
g) a heavy chain variable region comprising SEQ ID NO: 74 and a light chain variable region comprising SEQ ID NO: 76;
h) a heavy chain variable region comprising SEQ ID NO: 78 and a light chain variable region comprising SEQ ID NO: 80;
i) a heavy chain variable region comprising SEQ ID NO: 82 and a light chain variable region comprising SEQ ID NO: 84;
j) a heavy chain variable region comprising SEQ ID NO: 86 and a light chain variable region comprising SEQ ID NO: 88;
k) a heavy chain variable region comprising SEQ ID NO: 101 and a light chain variable region comprising SEQ ID NO: 103;
l) a heavy chain variable region comprising SEQ ID NO: 109 and a light chain variable region comprising SEQ ID NO: 111;
m) a heavy chain variable region comprising SEQ ID NO: 118 and a light chain variable region comprising SEQ ID NO: 120 or SEQ ID NO: 221;
n) a heavy chain variable region comprising SEQ ID NO: 122 and a light chain variable region comprising SEQ ID NO: 124;
o) a heavy chain variable region comprising SEQ ID NO: 132 and a light chain variable region comprising SEQ ID NO: 134;
p) a heavy chain variable region comprising SEQ ID NO: 141 and a light chain variable region comprising SEQ ID NO: 143;
q) a heavy chain variable region comprising SEQ ID NO: 146 and a light chain variable region comprising SEQ ID NO: 148;
r) a heavy chain variable region comprising SEQ ID NO: 156 and a light chain variable region comprising SEQ ID NO: 158;
s) a heavy chain variable region comprising SEQ ID NO: 164 and a light chain variable region comprising SEQ ID NO: 166;
t) a heavy chain variable region comprising SEQ ID NO: 174 and a light chain variable region comprising SEQ ID NO: 176;
U) a heavy chain variable region comprising SEQ ID NO: 177 and a light chain variable region comprising SEQ ID NO: 180;
v) a heavy chain variable region comprising SEQ ID NO: 178 and a light chain variable region comprising SEQ ID NO: 180;
w) a heavy chain variable region comprising SEQ ID NO: 179 and a light chain variable region comprising SEQ ID NO: 180;
x) a heavy chain variable region comprising SEQ ID NO: 177 and a light chain variable region comprising SEQ ID NO: 181;
y) a heavy chain variable region comprising SEQ ID NO: 178 and a light chain variable region comprising SEQ ID NO: 181;
z) a heavy chain variable region comprising SEQ ID NO: 179 and a light chain variable region comprising SEQ ID NO: 181;
aa) a heavy chain variable region comprising SEQ ID NO: 177 and a light chain variable region comprising SEQ ID NO: 182;
bb) a heavy chain variable region comprising SEQ ID NO: 178 and a light chain variable region comprising SEQ ID NO: 182;
cc) a heavy chain variable region comprising SEQ ID NO: 179 and a light chain variable region comprising SEQ ID NO: 182;
dd) a heavy chain variable region comprising SEQ ID NO: 177 and a light chain variable region comprising SEQ ID NO: 183;
ee) a heavy chain variable region comprising SEQ ID NO: 178 and a light chain variable region comprising SEQ ID NO: 183;
ff) a heavy chain variable region comprising SEQ ID NO: 179 and a light chain variable region comprising SEQ ID NO: 183;
gg) a heavy chain variable region comprising SEQ ID NO: 189 and a light chain variable region comprising SEQ ID NO: 195;
hh) a heavy chain variable region comprising SEQ ID NO: 189 and a light chain variable region comprising SEQ ID NO: 196;
ii) a heavy chain variable region comprising SEQ ID NO: 189 and a light chain variable region comprising SEQ ID NO: 197;
jj) a heavy chain variable region comprising SEQ ID NO: 189 and a light chain variable region comprising SEQ ID NO: 198;
kk) a heavy chain variable region comprising SEQ ID NO: 189 and a light chain variable region comprising SEQ ID NO: 199;
ll) a heavy chain variable region comprising SEQ ID NO: 190 and a light chain variable region comprising SEQ ID NO: 200;
mm) a heavy chain variable region comprising SEQ ID NO: 190 and a light chain variable region comprising SEQ ID NO: 201;
nn) a heavy chain variable region comprising SEQ ID NO: 190 and a light chain variable region comprising SEQ ID NO: 202;
oo) a heavy chain variable region comprising SEQ ID NO: 190 and a light chain variable region comprising SEQ ID NO: 203;
pp) a heavy chain variable region comprising SEQ ID NO: 191 and a light chain variable region comprising SEQ ID NO: 200;
qq) a heavy chain variable region comprising SEQ ID NO: 191 and a light chain variable region comprising SEQ ID NO: 201;
rr) a heavy chain variable region comprising SEQ ID NO: 191 and a light chain variable region comprising SEQ ID NO: 202;
ss) a heavy chain variable region comprising SEQ ID NO: 191 and a light chain variable region comprising SEQ ID NO: 203;
tt) a heavy chain variable region comprising SEQ ID NO: 192 and a light chain variable region comprising SEQ ID NO: 200;
uu) a heavy chain variable region comprising SEQ ID NO: 192 and a light chain variable region comprising SEQ ID NO: 201;
vv) a heavy chain variable region comprising SEQ ID NO: 192 and a light chain variable region comprising SEQ ID NO: 202;
ww) a heavy chain variable region comprising SEQ ID NO: 192 and a light chain variable region comprising SEQ ID NO: 203;
xx) a heavy chain variable region comprising SEQ ID NO: 193 and a light chain variable region comprising SEQ ID NO: 200;
yy) a heavy chain variable region comprising SEQ ID NO: 193 and a light chain variable region comprising SEQ ID NO: 201;
zz) a heavy chain variable region comprising SEQ ID NO: 193 and a light chain variable region comprising SEQ ID NO: 202;
aaa) a heavy chain variable region comprising SEQ ID NO: 193 and a light chain variable region comprising SEQ ID NO: 203;
bbb) a heavy chain variable region comprising SEQ ID NO: 194 and a light chain variable region comprising SEQ ID NO: 200;
ccc) a heavy chain variable region comprising SEQ ID NO: 194 and a light chain variable region comprising SEQ ID NO: 201;
ddd) a heavy chain variable region comprising SEQ ID NO: 194 and a light chain variable region comprising SEQ ID NO: 202; or
eee) a heavy chain variable region comprising SEQ ID NO: 194 and a light chain variable region comprising SEQ ID NO: 203.

In some embodiments, the antibody or antigen-binding fragment thereof provided herein further comprises one or more amino acid residue substitutions or modifications yet retains specific binding affinity to human CD24 or cynomolgus monkey CD24.

In some embodiments, the substitution is in one or more CDR sequences, and/or in one or more of the VH or VL sequences but not in any of the CDR sequences.

In some embodiments, the antibody or antigen-binding fragment thereof provided herein further comprises an immunoglobulin constant region, optionally a constant region of human Ig, or optionally a constant region of human IgG.

In some embodiments, the constant region comprises a constant region of human IgG1, IgG2, IgG3, or IgG4.

In some embodiments, the antibody or antigen-binding fragment thereof provided herein is a chimeric or humanized.

In some embodiments, the antibody or antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

In some embodiments, the antibody or antigen-binding fragment thereof provided herein is bispecific.

In some embodiments, the antibody or antigen-binding fragment thereof provided herein capable of specifically binding to a first and a second epitope of CD24, or capable of specifically binding to CD24 and a second antigen.

In some embodiments, the antibody or antigen-binding fragment thereof provided herein binds the second antigen via a Fab or a scFv, optionally, binds CD24 via a Fab and binds the second antigen via a scFv.

In some embodiments, the bispecific antibody molecule comprises a heavy chain in the format of: VH(anti-CD24)-CH1-Hinge-CH2-CH3-spacer-second antigen-binding scFv, associated with a light chain in the format of VL(anti-CD24)-CL.

In some embodiments, the second antigen is an immune related target, optionally selected from the group consisting of: PD-L1, PD-L2, PD-1, CLTA-4, TIM-3, LAG3, CD160, 2B4, TGF β, VISTA, BTLA, TIGIT, LAIR1, OX40, CD2, CD27, ICAM-1, NKG2C, SLAMF7, NKp80, CD160, B7-H3, LFA-1, ICOS, 4-1BB, GITR, CD30, CD40, BAFFR, HVEM, CD7, LIGHT, IL-2, IL-15, CD3, CD16, SIRPα, Siglec 10, LILRB2, Clever-1, Macro, LILRB4, Siglec15, CSF1R, PSGL-1, VSIG-4, B2M and CD83.

In some embodiments, the second antigen comprises a tumor antigen.

In some embodiments, the tumor antigen comprises CA-125, gangliosides G(D2), G(M2) and G(D3), CD19, CD20, CD33, CD47, CD52, Ep-CAM, CEA, CLDN18.2, bombesin-like peptides, PSA, HER2/neu, epidermal growth factor receptor (EGFR), erbB2, erbB3/HER3, erbB4, CD44v6, CD44v9, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R, CDH6, CEA, FOLR1, TROP2, PTK7, SLITRK6, CD142, NECTIN-4, ROR1, ROR2, CD142, CD123, CD22, CD79b, DLL3, SLC family or CO17-1A.

In some embodiments, the second antigen is an immunoinhibitory molecule selected from the group consisting of PD-L1, SIRPα, CD47 and B2M.

In some embodiments, the tumor antigen is CD47.

In some embodiments, the antibody or antigen-binding fragment is linked to one or more conjugates, optionally, wherein the conjugate is covalently attached either directly or via a linker.

In some embodiments, the conjugate comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In some embodiments, the antibody or antigen-binding fragment is capable of specifically binding to CD24, and optionally wherein the CD24 are derived from human or cynomolgus monkey, and optionally wherein the CD24 is a recombinant CD24 or a CD24 expressed on a cell surface.

In one aspect, the present disclosure provides an antibody or antigen-binding fragment thereof, which competes for the same epitope with the antibody or antigen-binding fragment thereof provided herein.

In some embodiments, the antibody or antigen-binding fragment thereof provided herein competes for the same epitope with the antibody or antigen-binding fragment thereof comprising: a heavy chain variable region comprising HCDR1, HCDR2, and HCDR3 of SEQ ID NO: 104, SEQ ID NO: 105, and SEQ ID NO: 106 respectively; and a light chain variable region comprising LCDR1, LCDR2, and LCDR3, wherein the LCDR1 comprises an amino acid sequence selected from the group consisting of SEQ ID NO: 107, 184, 185, 186, 187 and 188, the LCDR2 comprises an amino acid sequence of SEQ ID NO: 108, and the LCDR3 comprises an amino acid sequence of SEQ ID NO: 37.

In some embodiments, the antibody or antigen-binding fragment thereof provided herein competes for the same epitope with the antibody or antigen-binding fragment thereof comprising: a heavy chain variable region comprising HCDR1, HCDR2, and HCDR3 of SEQ ID NO: 95, SEQ ID NO: 96, and SEQ ID NO: 97 respectively; and a light chain variable region comprising LCDR1, LCDR2, and LCDR3 of SEQ ID NO: 98, SEQ ID NO: 99, and SEQ ID NO: 100 respectively.

In some embodiments, the antibody or antigen-binding fragment thereof provided herein selectively binds to CD24 expressed in a cancer cell over a non-cancer cell.

In one aspect, the present disclosure provides a pharmaceutical composition comprising the antibody or antigen-binding fragment thereof provided herein, and a pharmaceutically acceptable carrier.

In some embodiments, the pharmaceutical composition provided herein further comprises a second therapeutic agent.

In some embodiments, the second therapeutic agent is an antagonist against one or more immunoinhibitory molecules.

In some embodiments, the second therapeutic agent is a CD47 antagonist.

In some embodiments, the CD47 antagonist is a SIRPα-Fc fusion protein or variant thereof, or an anti-CD47 antibody or antigen-binding fragment thereof.

In one aspect, the present disclosure provides an isolated polynucleotide encoding the antibody or antigen-binding fragment thereof provided herein.

In some embodiments, the isolated polynucleotide provided herein comprises a nucleotide sequence selecting from a group consisting of SEQ ID NO. 51, 53, 55, 57, 59, 61, 63, 65, 67, 69, 71, 73, 75, 77, 79, 81, 83, 85, 87, 89, 102, 110, 204, 205, 206, 207, 208, 209, 210, 211, 212, 213, 214, 215, 216, 217, 218, 219 and 220.

In one aspect, the present disclosure provides a vector comprising the isolated polynucleotide provided herein.

In one aspect, the present disclosure provides a host cell comprising the vector provided herein.

In one aspect, the present disclosure provides a method of expressing the antibody or antigen-binding fragment thereof provided herein, comprising culturing the host cell provided herein under the condition at which the vector provided herein is expressed

In one aspect, the present disclosure provides a method of treating a disease or condition in a subject that would benefit from modulation of CD24 activity, comprising administering to the subject a therapeutically effective amount of the antibody or antigen-binding fragment thereof provided herein or the pharmaceutical composition provided herein.

In some embodiments, the disease or condition is a CD24 related disease or condition.

In some embodiments, the disease or condition is cancer, adaptive immune disease, autoimmune disease, inflammatory disease, or infectious disease.

In some embodiments, the cancer is lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, adenocarcinoma, leukemia, myeloma and lymphoma.

In some embodiments, the cancer is chemoresistant cancer.

In some embodiments, the disease or condition is hematological cancer chosen from B-cell lymphomas, such as Hodgkin's lymphoma, non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), diffuse large B cell lymphoma (DLBCL), Marginal zone B-cell lymphoma (MZL), Mantle cell lymphoma (MCL), Richter's syndrome, Burkitt's lymphoma or follicular lymphoma.

In some embodiments, the subject is human.

In some embodiments, the method provided herein comprises administering to the subject a therapeutically effective amount of a second therapeutic agent. In some embodiments, the second therapeutic agent is an antagonist against one or more immunoinhibitory molecules.

In some embodiments, the second therapeutic agent is a CD47 antagonist.

In some embodiments, the CD47 antagonist is a SIRPα-Fc fusion protein or variant thereof, or an anti-CD47 antibody or antigen-binding fragment thereof.

In some embodiments, the administration is via oral, nasal, intravenous, subcutaneous, sublingual, or intramuscular administration.

In one aspect, the present disclosure provides a method of modulating CD24 activity in a CD24-expressing cell, comprising exposing the CD24-expressing cell to the antibody or antigen-binding fragment thereof provided herein.

In one aspect, the present disclosure provides a method of detecting presence or amount of CD24 in a sample, comprising contacting the sample with the antibody or antigen-binding fragment thereof provided herein, and determining the presence or the amount of CD24 in the sample.

In one aspect, the present disclosure provides a method of diagnosing a CD24 related disease or condition in a subject, comprising: a) obtaining a sample from the subject; b) contacting the sample obtained from the subject with the antibody or antigen-binding fragment thereof provided herein; c) determining presence or amount of CD24 in the sample; and d) correlating the presence or the amount of CD24 to existence or status of the CD24 related disease or condition in the subject.

In one aspect, the present disclosure provides an use of the antibody or antigen-binding fragment thereof provided herein in the manufacture of a medicament for treating a CD24 related disease or condition in a subject.

In some embodiments, the medicament further comprises a second therapeutic agent. In some embodiments, the second therapeutic agent is an antagonist against one or more immunoinhibitory molecules.

In some embodiments, the second therapeutic agent is a CD47 antagonist. In some embodiments, the CD47 antagonist comprises a SIRPα-Fc fusion protein or variant thereof, and an anti-CD47 antibody or antigen-binding fragment thereof.

In one aspect, the present disclosure provides an use of the antibody or antigen-binding fragment thereof provided herein in the manufacture of a diagnostic reagent for diagnosing a CD24 related disease or condition.

In one aspect, the present disclosure provides a kit comprising the antibody or antigen-binding fragment thereof provided herein, useful in detecting CD24, optionally recombinant CD24, CD24 expressed on cell surface, or CD24-expressing cells.

In one aspect, the present disclosure provides a chimeric antigen receptor (CAR) comprising an antigen binding domain, a transmembrane domain, a costimulatory signaling region, and a TCR signaling domain, wherein the antigen binding domain specifically binds to CD24 and comprises an antigen binding fragment provided herein. In some embodiments, the antigen binding fragment is a Fab or a scFv.

In some embodiments, the CAR provided herein is bispecific.

In some embodiments, the CAR is capable of further specifically binding to a second antigen other than CD24, or a second epitope on CD24. In some embodiments, the second antigen is a tumor antigen.

In some embodiments, the TCR signaling domain is selected from the group consisting of: an intracellular signal regions sequence of CD3ζ, FccRI γ, CD27, CD28, CD137, CD134, MyD88, CD40, CD278, TLRs, or a combination thereof.

In some embodiments, the transmembrane region comprises a transmembrane region of CD3, CD4, CD8 or CD28.

In one aspect, the present disclosure provides a nucleic acid sequence encoding the chimeric antigen receptor (CAR) provided herein. In one aspect, the present disclosure provides a cell comprising the nucleic acid sequence provided herein. In one aspect, the present disclosure provides a vector comprising the nucleic acid sequence provided herein.

In one aspect, the present disclosure provides a cell genetically modified to express the CAR provided herein.

In some embodiments, the cell is immune cell, optionally, wherein the immune cell is T lymphocyte, NK cell, monocyte, macrophage or NKT lymphocyte.

In one aspect, the present disclosure provides a method for stimulating a T cell-mediated immune response to a CD24-expressing cell or tissue in a mammal, the method comprising administering to the mammal an effective amount of a cell genetically modified to express the CAR provided herein.

In one aspect, the present disclosure provides a method of treating a mammal having a CD24 related disease or condition, comprising administering to the mammal an effective amount of a cell provided herein, thereby treating the mammal.

In some embodiments, the cell is an autologous T cell.

In some embodiments, the CD24 related disease or condition is cancer.

In some embodiments, the mammal is a human subject.

In one aspect, the present disclosure provides a method of treating cancer in a subject in need thereof, comprising administering an anti-CD24 antibody or antigen-binding fragment thereof that only binds to the glycosylated or sialylated CD24, preferably, only binds to the sialic acid of CD24.

In some embodiments, the anti-CD24 antibody or antigen-binding fragment thereof selectively binds to CD24 expressed in a cancer cell over a non-cancer cell.

In some embodiments, the anti-CD24 antibody or antigen-binding fragment thereof competes for the same epitope with the antibody or antigen-binding fragment thereof comprising: a heavy chain variable region comprising HCDR1, HCDR2, and HCDR3 of SEQ ID NO: 104, SEQ ID NO: 105, and SEQ ID NO: 106 respectively; and a light chain variable region comprising LCDR1, LCDR2, and LCDR3, wherein the LCDR1 comprises an amino acid sequence selected from the group consisting of SEQ ID NO: 107, 184, 185, 186, 187 and 188, the LCDR2 comprises an amino acid sequence of SEQ ID NO: 108, and the LCDR3 comprises an amino acid sequence of SEQ ID NO: 37, or comprising: a heavy chain variable region comprising HCDR1, HCDR2, and HCDR3 of SEQ ID NO: 95, SEQ ID NO: 96, and SEQ ID NO: 97 respectively; and a light chain variable region comprising LCDR1, LCDR2, and LCDR3 of SEQ ID NO: 98, SEQ ID NO: 99, and SEQ ID NO: 100 respectively.

In some embodiments, the anti-CD24 antibody or antigen-binding fragment thereof comprises: a heavy chain variable region comprising HCDR1, HCDR2, and HCDR3 of SEQ ID NO: 104, SEQ ID NO: 105, and SEQ ID NO: 106 respectively; and a light chain variable region comprising LCDR1, LCDR2, and LCDR3, wherein the LCDR1 comprises an amino acid sequence selected from the group consisting of SEQ ID NO: 107, 184, 185, 186, 187 and 188, the LCDR2 comprises an amino acid sequence of SEQ ID NO: 108, and the LCDR3 comprises an amino acid sequence of SEQ ID NO: 37, or comprising: a heavy chain variable region comprising HCDR1, HCDR2, and HCDR3 of SEQ ID NO: 95, SEQ ID NO: 96, and SEQ ID NO: 97 respectively; and a light chain variable region comprising LCDR1, LCDR2, and LCDR3 of SEQ ID NO: 98, SEQ ID NO: 99, and SEQ ID NO: 100 respectively.

### BRIEF DESCRIPTION OF FIGURES

FIG. 1A shows the MFI of the chimeric anti-CD24 antibodies provided herein and bench mark antibody SN3 on 293F blank cells.

FIG. 1B shows the MFI of the chimeric anti-CD24 antibodies provided herein and bench mark antibody SN3 on 293F-hCD24 cells.

FIG. 1C shows the MFI of the chimeric anti-CD24 antibodies provided herein and bench mark antibody SN3 on MCF7 cells.

FIG. 1D shows the MFI of the chimeric anti-CD24 antibodies provided herein and bench mark antibody SN3 on MDA-MB-468 cells.

FIG. 1E shows the MFI of the chimeric anti-CD24 antibodies provided herein and bench mark antibody SN3 on Nalm1 cells.

FIG. 1F shows the MFI of the chimeric anti-CD24 antibodies provided herein and bench mark antibody SN3 on CHOK1-hCD24 cells.

FIG. 1G shows the MFI of the chimeric anti-CD24 antibodies provided herein and bench mark antibody SN3 on HT-29 cells.

FIG. 1H shows the MFI of the chimeric anti-CD24 antibodies provided herein and bench mark antibody SN3 on NCI H1975 cells.

FIG. 1I shows the MFI of the chimeric anti-CD24 antibodies provided herein and bench mark antibody SN3 on A549 cells.

FIG. 1J shows the MFI of the chimeric anti-CD24 antibodies provided herein on Nalm6 cells.

FIG. 1K shows the MFI of the chimeric anti-CD24 antibodies provided herein and bench mark antibody SN3 on SU-DHL6 cells.

FIG. 1L shows the MFI of the chimeric anti-CD24 antibodies provided herein on human B cells.

FIG. 1M shows the MFI of the chimeric anti-CD24 antibodies provided herein and bench mark antibody SN3 on DoHH2 cells.

FIG. 1N shows the MFI of the chimeric anti-CD24 antibodies provided herein and bench mark antibody SN3 on HT29 cells.

FIG. 1O shows the MFI of the chimeric anti-CD24 antibodies provided herein on Huh7 cells.

FIG. 1P shows the MFI of the chimeric anti-CD24 antibodies provided herein and bench mark antibody SN3 on MCF7 cells.

FIG. 1Q shows the MFI of the chimeric anti-CD24 antibodies provided herein and bench mark antibody SN3 on a549 cells.

FIG. 1R shows the MFI of the chimeric anti-CD24 antibodies provided herein and bench mark antibody SN3 on NCI-H1975 cells.

FIG. 2A shows the phagocytosis index of chimeric anti-CD24 antibodies and reference antibody SN3 on 293F-hCD24 cells.

FIG. 2B shows the phagocytosis index of chimeric anti-CD24 antibodies and reference antibody SN3 on HT-29 cells.

FIG. 3A shows the phagocytosis index of combination treatment of anti-CD47 antibody hu5F9 with chimeric anti-CD24 antibody chAb110D4G4 on HT29 coleretal tumor cells.

FIG. 3B shows the phagocytosis index of combination treatment of anti-CD47 antibody hu5F9 with chimeric anti-CD24 antibody chAb111F3A2 on HT29 coleretal tumor cells.

FIG. 3C shows the phagocytosis index of combination treatment of anti-CD47 antibody hu5F9 with chimeric anti-CD24 antibody chAb81A1F8 on HT29 coleretal tumor cells.

FIG. 4A shows the MFI of hIgG4 alone, ch81A1F8-IgG4 alone, bispecific antibody (ch81A1F8-IgG4+Hu5F9 derived scFv) and bench mark antibody SN3 on HT29 cells.

FIG. 4B shows the phagocytosis index of hIgG4 alone, hu5F9 alone, ch81A1F8-IgG4 alone, bispecific antibody (ch81A1F8-IgG4+Hu5F9 derived scFv) as well as the combination of anti-CD24 antibody ch81A1F8-IgG4 and anti-CD47 antibody Hu5F9 (“Combo”) on HT29 cells.

FIG. 5A shows the MFI of the chimeric anti-CD24 antibody 81A1F8 and the humanized 81A1F8 antibodies on HT-29.

FIG. 5B shows the MFI of the chimeric anti-CD24 antibody 81A1F8 and the humanized 81A1F8 antibodies on Nalm6.

FIG. 5C shows the MFI of the chimeric anti-CD24 antibody 101H9G9A2 and the humanized 101H9G9A2 antibodies on HT-29.

FIG. 5D shows the KD of the chimeric anti-CD24 antibody 81A1F8 and the humanized 81A1F8 antibodies as measured using the Gatorprime system.

FIGS. 6A-6D show the MFI of the chimeric anti-CD24 antibodies provided herein and/or bench mark antibody SN3 on cells digested with neuraminidase or de-glycosylation.

FIG. 7A shows the KD of the chimeric anti-CD24 antibodies provided herein and reference antibody SWA11 on mFc tagged human CD24 as measured by BIAcore.

FIG. 7B shows the KD of the chimeric anti-CD24 antibody 81A1F8 and the humanized 81A1F8 antibodies provided herein on mFc tagged human CD24 as measured by BIAcore.

FIG. 8 shows the normalized OD450 of the chimeric anti-CD24 antibody 81A1F8 of different concentrations on mFc tagged human CD24 as measured by ELISA.

FIG. 9A shows the phagocytosis index of the chimeric anti-CD24 antibodies, reference antibody SWA11, and anti-CD47 antibody hu5F9-IgG4 on HT-29 cells.

FIG. 9B shows the phagocytosis index of the chimeric anti-CD24 antibodies, reference antibody SWA11, and anti-CD47 antibody hu5F9-IgG4 on Nalm6 cells.

FIG. 9C shows the phagocytosis index of 81A1F8-LALAPG antibodies with rare Fc dependent functions, alone or in combination with Rituximab on SU-DHL-6 cells.

FIG. 9D shows the phagocytosis index of the anti-CD24 antibody 81A1F8 alone, the anti-CD47 antibody Hu5F9 alone, as well as the combination of the anti-CD24 antibody 81A1F8 and the anti-CD47 antibody Hu5F9 on HT-29 cells.

FIG. 10A. shows the ADCC effect induced by the chimeric anti-CD24 antibody 81A1F8 and the humanized 81A1F8 antibodies on HT-29 cells.

FIG. 10B shows the CDC effect induced by the chimeric anti-CD24 antibody 81A1F8 and the humanized 81A1F8 antibodies on hCD24-MCF7 cells.

FIG. 11A shows the tumor volumes after the administration of the chimeric anti-CD24 antibody 81A1F8 alone, Oxaliplatin alone, as well as the combination of the chimeric anti-CD24 antibody 81A1F8 and Oxaliplatin in C57BL/6J syngeneic mice model.

FIG. 11B shows the body weights after the administration of the chimeric anti-CD24 antibody 81A1F8 alone, Oxaliplatin alone, as well as the combination of the chimeric anti-CD24 antibody 81A1F8 and Oxaliplatin in C57BL/6J syngeneic mice model.

FIG. 11C shows summary of the in vivo efficacy test of the chimeric anti-CD24 antibody 81A1F8 alone, Oxaliplatin alone, as well as the combination of the chimeric anti-CD24 antibody 81A1F8 and Oxaliplatin in C57BL/6J syngeneic mice model.

FIG. 11D shows CD8/CD4 ratio and M1/M2 macrophage ratio induced by the combination treatment of the chimeric anti-CD24 antibody 81A1F8 and Oxaliplatin in C57BL/6J syngeneic mice model.

FIG. 12A shows the tumor volumes after the administration of the chimeric anti-CD24 antibody 81 A1F8-mIgG2a at different dosages in MC38-hCD24 syngeneic model.

FIG. 12B shows the tumor volumes after the administration of the chimeric anti-CD24 antibody 81 A1F8-mIgG2a at different dosages and the chimeric anti-CD24 antibody 81A1F8-mIgG2a after rechallenged in MC38-hCD24 syngeneic model.

FIG. 12C shows the tumor growth curves for each mouse after the administration of chimeric anti-CD24 antibody 81A1F8-mIgG2a at different dosages in MC38-hCD24 syngeneic model.

FIG. 12D shows summary for the in vivo efficacy of the chimeric anti-CD24 antibody 81 A1F8-mIgG2a at different dosages.

FIG. 13A shows the tumor growth curves after the administration of single agent (81A1F8-2141-mIgG2a, Oxaliplatin or Atezolizumab) or combination strategy.

FIG. 13B shows the tumor growth curves for each mouse of the treatment groups.

FIG. 13C shows summary for the in vivo efficacy of single agent (81A1F8-2141-mIgG2a, Oxaliplatin or Atezolizumab) or combination strategy.

FIG. 14A shows the tumor growth curves after the administration of single agent (101H9G9A2-mIgG2a, 81 A1F8-2141-mIgG2a, Oxaliplatin or Atezolizumab) or combination strategy.

FIG. 14B shows the body weights of the mice after the administration of single agent (101H9G9A2-mIgG2a, 81A1F8-2141-mIgG2a, Oxaliplatin or Atezolizumab) or combination strategy.

FIG. 14C shows summary for the in vivo efficacy of single agent (101H9G9A2-mIgG2a, 81A1F8-2141-mIgG2a, Oxaliplatin or Atezolizumab) or combination strategy.

FIG. 15 shows the MFI of the anti-cmCD24 antibodies provided herein on cmCD24-293F cells.

FIG. 16A shows the phagocytosis index of the anti-cmCD24 antibodies provided herein on cynoCD24-293F cells.

FIG. 16B shows the ADCC effect induced by the anti-cmCD24 antibodies provided herein on cmCD24-293F cells.

FIG. 16C shows the CDC effect induced by the anti-cmCD24 antibodies provided herein on cynoCD24-293F cells.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, and HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J.Mol.Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antibody molecule” as used herein refers to an antigen-binding protein or polypeptide comprising at least one antibody fragment (such as CDR, and/or variable region sequence). An antibody molecule includes, for example, a monoclonal antibody, an antibody fragment or domain, a fusion protein comprising an antibody fragment or domain, a polypeptide complex comprising an antibody fragment or domain, and so on.

The term “antigen-binding domain” (e.g. CD24-binding domain) as used herein refers to an antibody fragment formed from a portion of an antibody comprising one or more CDRs, or any other antibody fragment that binds to an antigen but does not comprise an intact native antibody structure. Examples of antigen-binding domain include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding domain is capable of binding to the same antigen to which the parent antibody binds. In certain embodiments, an antigen-binding domain may comprise one or more CDRs from a particular human antibody grafted to a framework region from one or more different human antibodies. For more and detailed formats of antigen-binding domain are described in Spiess et al, 2015, and Brinkman et al., mAbs, 9(2), pp. 182-212 (2017), which are incorporated herein by entirety reference.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

A “fragment difficult (Fd)” with regard to an antibody refers to the amino-terminal half of the heavy chain fragment that can be combined with the light chain to form Fab. For example, Fd fragment may consists of the VH and CH1 domains

“Fv” with regard to an antibody refers to the smallest fragment of the antibody to bear the complete antigen-binding site. An Fv fragment consists of the variable region of a single light chain bound to the variable region of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), and complement dependent cytotoxicity (CDC), but does not function in antigen binding.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes).

In certain embodiments, a “bispecific ds diabody” is a diabody target two different antigens (or epitopes).

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'S of the other moiety and form two binding sites which can target the same antigens (or epitopes) or different antigens (or epitopes).

In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “chimeric” as used herein, means an antibody or antigen-binding domain, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding domain comprises CDRs derived from non-human animals, FR regions derived from human, and when applicable, the constant regions derived from human.

The term “operably link” or “operably linked” refers to a juxtaposition, with or without a spacer or a linker or an intervening sequence, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. For another example, an antigen-binding domain can be operably linked to another antigen-binding domain with an intervening sequence there between, and such intervening sequence can be a spacer or can comprise a much longer sequence such as a constant region of an antibody. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

An “antigen” as used herein refers to a compound, composition, peptide, polypeptide, protein or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

The term “CD24” as used herein is intended to encompass any form of CD24, for example, 1) native unprocessed CD24 molecule, “full-length” CD24 chain or naturally occurring variants of CD24, including, for example, splice variants or allelic variants; 2) any form of CD24 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CD24 subunit generated through recombinant method.

The term “anti-CD24 antibody”, “anti-CD24 binding domain” or “CD24-binding domain” refers to an antibody or antigen-binding domain that is capable of specifically binding to CD24 (e.g. human, mouse or cynomolgus monkey CD24).

The term “specific binding” or “specifically binds” as used herein refers to a non-random binding reaction between two molecules, such as for example between an antibody and an antigen.

Binding of the antibodies to recombinant CD24 or CD24 expressed on surface of cells can also be represented by “half maximal effective concentration” (EC50) value, which refers to the concentration of an antibody where 50% of its maximal effect (e.g., binding or inhibition etc.) is observed. The EC50 value can be measured by methods known in the art, for example, sandwich assay such as ELISA, Western Blot, flow cytometry assay, and other binding assay. In certain embodiments, the antibodies and the fragments thereof provided herein specifically bind to recombinant human CD24 at an EC50 (i.e. 50% binding concentration) of no more than 0.05 nM, no more than 0.06 nM, no more than 0.07 nM, no more than 0.08 nM, no more than 0.09 nM, no more than 0.1 nM, no more than 0.2 nM, no more than 0.3 nM, no more than 0.4 nM, no more than 0.5 nM, no more than 0.6 nM, no more than 0.7 nM, no more than 0.8 nM, no more than 0.9 nM, no more than 1 nM, no more than 2 nM, no more than 3 nM, no more than 4 nM, no more than 5 nM, no more than 6 nM, no more than 7 nM, no more than 8 nM, no more than 9 nM, no more than 10 nM, no more than 20 nM, no more than 25 nM, no more than 30 nM, no more than 35 nM, no more than 40 nM, no more than 50 nM, no more than 60 nM, no more than 70 nM, no more than 80 nM, no more than 90 nM, no more than 100 nM, no more than 110 nM, no more than 120 nM, no more than 122 nM, no more than 124 nM, no more than 126 nM, no more than 128 nM, no more than 130 nM, no more than 132 nM, no more than 134 nM, no more than 136 nM, no more than 138 nM, no more than 140 nM, no more than 200 nM, no more than 210 nM, no more than 250 nM, no more than 300 nM, or no more than 400 nM by flow cytometry assay.

The ability to “block binding” or “compete for the same epitope” as used herein refers to the ability of an antibody or antigen-binding domain to inhibit the binding interaction between two molecules (e.g. human CD24 and Siglec-10) to any detectable degree. In certain embodiments, an antibody or antigen-binding domain that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation. Two antibodies may bind the same or a closely related epitope within an antigen if they exhibit competitive binding for the antigen. For example, if an antibody or antigen-binding domain blocks binding of a reference antibody to the antigen by at least 85%, or at least 90%, or at least 95%, then the antibody or antigen-binding domain may be considered to bind the same/closely related epitope as the reference antibody.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “homolog” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex, Fc receptor and effector cell (e.g., macrophage). Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody-dependent cellular phagocytosis (ADCP) induced by binding of Fc region of an antibody to phagocytosis. It has become well established that the specific glycan structures associated with the conserved bi-antennary glycan in the Fc-CH2 domain can strongly influence the interaction with the FcyRs that mediate ADCC and ADCP and with C1q binding, the initial binding event leading to CDC (see Reusch D, Tejada M L. Fc glycans of therapeutic antibodies as critical quality attributes. Glycobiology 2015; 25: 1325-34).

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector.

The phrase “host cell” as used herein refers to a cell into which an exogenous polynucleotide and/or a vector has been introduced.

A “CD24-related” disease or condition as used herein refers to any disease or condition caused by, exacerbated by, or otherwise linked to increased or decreased expression or activities of CD24. In some embodiments, the CD24 related condition is immune-related disorder, such as, for example, cancer, autoimmune disease, inflammatory disease or infectious disease.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

The term “chemoresistant cancer” as used herein refers to a type of cancer that are not responsive to the effects of chemotherapy. For example, a cancer that has been responding to a chemotherapy or a combination of different chemotherapies suddenly begins to grow can be referred to as a chemoresistant cancer.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Anti-CD24 Antibody

The present disclosure provides anti-CD24 antibodies and antigen-binding fragments thereof comprising one or more (e.g. 1, 2, 3, 4, 5, or 6) CDR sequences of an anti-CD24 antibody 33E7E12, 71A4F12, 56A7E3, 58F10-1F6, 73H5-1G2, 110D4G4, 81A1F8, 100F2E3, 111F3A2, 81A7A10, 107D10D11, 101H9G9A2, 173B1C1, 107D10E11, 109G10A6, 94G12D11, 92F5B8, 185B11E3, 168B11E2 or 188H6D3. In certain embodiments, the anti-CD24 antibodies and antigen-binding fragments thereof provided herein is capable of specifically binding to CD24. Optionally, the CD24 are derived from human or cynomolgus monkey. In certain embodiments, the CD24 is a recombinant CD24 or a CD24 expressed on a cell surface.

“33E7E12” as used herein refers to a mouse monoclonal antibody having a heavy chain variable region of SEQ ID NO: 50, and a light chain variable region of SEQ ID NO: 52.

“71A4F12” as used herein refers to a mouse monoclonal antibody having a heavy chain variable region of SEQ ID NO: 54, and a light chain variable region of SEQ ID NO: 56.

“56A7E3” as used herein refers to a mouse monoclonal antibody having a heavy chain variable region of SEQ ID NO: 58, and a light chain variable region of SEQ ID NO: 64.

“58F10-1F6” as used herein refers to a mouse monoclonal antibody having a heavy chain variable region of SEQ ID NO: 62, and a light chain variable region of SEQ ID NO: 64.

“73H5-1G2” as used herein refers to a mouse monoclonal antibody having a heavy chain variable region of SEQ ID NO: 66, and a light chain variable region of SEQ ID NO: 68.

“110D4G4” as used herein refers to a mouse monoclonal antibody having a heavy chain variable region of SEQ ID NO: 70, and a light chain variable region of SEQ ID NO: 72.

“81A1F8” as used herein refers to a mouse monoclonal antibody having a heavy chain variable region of SEQ ID NO: 74, and a light chain variable region of SEQ ID NO: 76.

“100F2E3” as used herein refers to a mouse monoclonal antibody having a heavy chain variable region of SEQ ID NO: 78, and a light chain variable region of SEQ ID NO: 80.

“111F3A2” as used herein refers to a mouse monoclonal antibody having a heavy chain variable region of SEQ ID NO: 82, and a light chain variable region of SEQ ID NO: 84.

“81A7A10” as used herein refers to a mouse monoclonal antibody having a heavy chain variable region of SEQ ID NO: 86, and a light chain variable region of SEQ ID NO: 88.

“107D10D11” as used herein refers to a mouse monoclonal antibody having a heavy chain variable region of SEQ ID NO: 101, and a light chain variable region of SEQ ID NO: 103.

“101H9G9A2” as used herein refers to a mouse monoclonal antibody having a heavy chain variable region of SEQ ID NO: 109, and a light chain variable region of SEQ ID NO: 111.

“173B1C1” as used herein refers to a mouse monoclonal antibody having a heavy chain variable region of SEQ ID NO: 118, and a light chain variable region of SEQ ID NO: 120 or SEQ ID NO: 221.

“107D11E11” as used herein refers to a mouse monoclonal antibody having a heavy chain variable region of SEQ ID NO: 122, and a light chain variable region of SEQ ID NO: 124.

“109G10A6” as used herein refers to a mouse monoclonal antibody having a heavy chain variable region of SEQ ID NO: 132, and a light chain variable region of SEQ ID NO: 134.

“94G12D11” as used herein refers to a mouse monoclonal antibody having a heavy chain variable region of SEQ ID NO: 141, and a light chain variable region of SEQ ID NO: 143.

“92F5B8” as used herein refers to a mouse monoclonal antibody having a heavy chain variable region of SEQ ID NO: 146, and a light chain variable region of SEQ ID NO: 146.

“185B11E3” as used herein refers to a mouse monoclonal antibody having a heavy chain variable region of SEQ ID NO: 156, and a light chain variable region of SEQ ID NO: 158.

“168B11E2” as used herein refers to a mouse monoclonal antibody having a heavy chain variable region of SEQ ID NO: 164, and a light chain variable region of SEQ ID NO: 166.

“188H6D3” as used herein refers to a mouse monoclonal antibody having a heavy chain variable region of SEQ ID NO: 174, and a light chain variable region of SEQ ID NO: 176.

Table 1 shows the CDR sequences of these 20 anti-CD24 antibodies according to IMGT numbering. The heavy chain and light chain variable region sequences are also provided below.

TABLE 1

| Antibody ID: | | CDR1 | CDR2 | CDR3 |
|---|---|---|---|---|
| 33E7E12 | VH | SEQ ID NO: 1 | SEQ ID NO: 3 | SEQ ID NO: 5 |
| | | NSWMN | QIQLKSDNYATR | GTDY |
| | | | YVESVRG | |
| 33E7E12 | VL | SEQ ID NO: 2 | SEQ ID NO: 4 | SEQ ID NO: 6 |
| | | KSSQSLLFSNGKTY | LVSNLDS | VQGTHFPYT |
| | | LN | | |
| 71A4F12 | VH | SEQ ID NO: 7 | SEQ ID NO: 9 | SEQ ID NO: 11 |
| | | AYNMD | DINPNNGDTVYN | RGSYYYGSSHY |
| | | | QNFKG | ALDF |
| 71A4F12 | VL | SEQ ID NO: 8 | SEQ ID NO: 10 | SEQ ID NO: 12 |
| | | RASQDIRNYLN | YTSRLLS | QQDNSLPRT |
| 56A7E3 | VH | SEQ ID NO: 7 | SEQ ID NO: 14 | SEQ ID NO: 11 |
| | | AYNMD | DINPINGGTIYNQ | RGSYYYGSSHY |
| | | | NFEG | ALDF |
| 56A7E3 | VL | SEQ ID NO: 13 | SEQ ID NO: 10 | SEQ ID NO: 15 |
| | | RASQDISNYLN | YTSRLLS | QQDHTLPRT |
| 58F10-1F6 | VH | SEQ ID NO: 16 | SEQ ID NO: 17 | SEQ ID NO: 18 |
| | | GYHMN | EINPITSGITYNQI | RDYGTSLDY |
| | | | FKA | |
| 58F10-1F6 | VL | SEQ ID NO: 13 | SEQ ID NO: 10 | SEQ ID NO: 15 |
| | | RASQDISNYLN | YTSRLLS | QQDHTLPRT |
| 73H5-1G2 | VH | SEQ ID NO: 19 | SEQ ID NO: 21 | SEQ ID NO: 23 |
| | | NYGVH | VIWRGGSTDYN | NYGYDCFAY |
| | | | AAFMS | |
| 73H5-1G2 | VL | SEQ ID NO: 20 | SEQ ID NO: 22 | SEQ ID NO: 24 |
| | | RASKSIIKYLA | SGSTLQS | QQHNEYPLT |
| 110D4G4 | VH | SEQ ID NO: 25 | SEQ ID NO: 27 | SEQ ID NO: 28 |
| | | GYYMN | EINPNTGDPRYD | RDYGTSL |
| | | | QKFKA | |
| 110D4G4 | VL | SEQ ID NO: 26 | SEQ ID NO: 22 | SEQ ID NO: 24 |
| | | RASKSISKYLAW | SGSTLQS | QQHNEYPLT |
| 81A1F8 | VH | SEQ ID NO: 25 | SEQ ID NO: 30 | SEQ ID NO: 28 |
| | | GYYMN | EINPNTGDTNNN | RDYGTSL |
| | | | QKFKA | |
| 81A1F8 | VL | SEQ ID NO: 29 | SEQ ID NO: 22 | SEQ ID NO: 31 |
| | | RASKSINKYLA | SGSTLQS | QQHNEYPIT |
| 100F2E3 | VH | SEQ ID NO: 32 | SEQ ID NO: 34 | SEQ ID NO: 36 |
| | | SYWMD | NIYPSDSKTHSN | RGGYYGYAMD |
| | | | QKFRD | Y |
| 100F2E3 | VL | SEQ ID NO: 33 | SEQ ID NO: 35 | SEQ ID NO: 37 |
| | | KSSQSLLNSGNQD | GASIRES | QNDHSYPYT |
| | | NYLA | | |
| 111F3A2 | VH | SEQ ID NO: 38 | SEQ ID NO: 40 | SEQ ID NO: 42 |
| | | TYGVS | VIWGDGTTNYH | YYGYPFFAY |
| | | | SALKP | |
| 111F3A2 | VL | SEQ ID NO: 39 | SEQ ID NO: 41 | SEQ ID NO: 43 |
| | | KASEDIYNRLA | GATNLVT | QQYWSSPPT |
| 81A7A10 | VH | SEQ ID NO: 44 | SEQ ID NO: 46 | SEQ ID NO: 48 |
| | | SYGVS | VIWGDGSTDYH | FYGYDEGFAY |
| | | | STLTS | |
| 81A7A10 | VL | SEQ ID NO: 45 | SEQ ID NO: 47 | SEQ ID NO: 49 |
| | | RASSTVNYMY | YTSRLAP | QQFTSSPLT |
| 107D10D11 | VH | SEQ ID NO: 95 | SEQ ID NO: 96 | SEQ ID NO: 97 |
| | | SYGVN | MIWSGGNTDYN | IWYYGMDY |
| | | | AAFIS | |
| 107D10D11 | VL | SEQ ID NO: 98 | SEQ ID NO: 99 | SEQ ID NO: 100 |
| | | KASQNVGTAVA | SASIRYP | LQYITFPR |
| 101H9G9A2 | VH | SEQ ID NO: 104 | SEQ ID NO: 105 | SEQ ID NO: 106 |
| | | DYTFTSYW | IYPSDSKT | ARRGGYYAYA |
| | | | | MDY |
| 101H9G9A2 | VI | SEQ ID NO: 107 | SEQ ID NO: 108 | SEQ ID NO: 37 |
| | | QSLLNSGNQDNY | GAR | QNDHSYPYT |
| 173B1C1 | VH | SEQ ID NO: 113 | SEQ ID NO: 114 | SEQ ID NO: 115 |
| | | SNTMH | YINPGSAYTNYN | LATYYDNDGY |
| | | | QKFND | AMDY |
| 173B1C1 | VL | SEQ ID NO: 13 | SEQ ID NO: 116 | SEQ ID NO: 117 |
| | | RASQDISNYLN | YTSKLHS | QQGHTLPWT |
| 107D10E11 | VH | SEQ ID NO: 222 | SEQ ID NO: 223 | SEQ ID NO: 224 |
| | | SYWIT | DISPAGGGRNYN | GDSTVDLDY |
| | | | ERFKN | |
| 107D10E11 | VL | SEQ ID NO: 225 | SEQ ID NO: 226 | SEQ ID NO: 227 |
| | | RSSKSLLHSNGNT | RVSNLAS | MQHLEYPY |
| | | YLY | | |
| 109G10A6 | VH | SEQ ID NO: 126 | SEQ ID NO: 127 | SEQ ID NO: 128 |
| | | DYNMD | DINPHNGDTIYN | RGAYYYGSSHY |
| | | | QKFKG | ALDF |
| 109G10A6 | VL | SEQ ID NO: 129 | SEQ ID NO: 130 | SEQ ID NO: 131 |
| | | RASQDITNYLN | YTSRLFS | QQGNTLPW |
| 94G12D11 | VH | SEQ ID NO: 25 | SEQ ID NO: 136 | SEQ ID NO: 137 |
| | | GYYMN | EINPNKGDSNLN | RDYGTSLDH |
| | | | QNFKA | |
| 94G12D11 | VL | SEQ ID NO: 138 | SEQ ID NO: 139 | SEQ ID NO: 140 |
| | | RASKSISKYLA | SGSTLHT | QQHNEYPI |
| 92F5B8 | VH | SEQ ID NO: 25 | SEQ ID NO: 30 | SEQ ID NO: 28 |
| | | GYYMN | EINPNTGDTNNN | RDYGTSL |
| | | | QKFKA | |
| 92FSB8 | VL | SEQ ID NO: 29 | SEQ ID NO: 145 | SEQ ID NO: 24 |
| | | RASKSINKYLA | SGSTLHS | QQHNEYPLT |
| 185B11E3 | VH | SEQ ID NO: 150 | SEQ ID NO: 151 | SEQ ID NO: 152 |
| | | GYTFTSNT | INPGSAYT | ARLATYYDNDG |
| | | | | YAMDY |
| 185B11E3 | VL | SEQ ID NO: 153 | SEQ ID NO: 154 | SEQ ID NO: 155 |
| | | SSVNY | GTS | FQGSGYPYT |
| 168B11E2 | VH | SEQ ID NO: 160 | SEQ ID NO: 161 | SEQ ID NO: 162 |
| | | GFSLTSYG | IWGDGNT | ANLYVLFAY |
| 168B11E2 | VL | SEQ ID NO: 163 | SEQ ID NO: 154 | SEQ ID NO: 155 |
| | | SSVSY | GTS | FQGSGYPYT |
| 188H6D3 | VH | SEQ ID NO: 168 | SEQ ID NO: 169 | SEQ ID NO: 170 |
| | | GYTFTNYW | INPTNGGT | ARSGGYDFDY |
| 188H6D3 | VL | SEQ ID NO: 171 | SEQ ID NO: 172 | SEQ ID NO: 173 |
| | | QSLLDSDGKTY | LVS | WQGTYFPLT |

Heavy or light chain variable region sequences of 33E7E12, 71A4F12, 56A7E3, 58F10-1F6, 73H5-1G2, 110D4G4, 81A1F8, 100F2E3, 111F3A2, 81A7A10, 107D10D11 101H9G9A2, 173B1C1, 107D10E11, 109G10A6, 94G12D11, 92F5B8, 185B11E3, 168B11E2 or 188H6D3 antibodies are provided below.

33E7E12-VH
Amino acid sequence (SEQ ID NO: 50):
EVKLEESGGGLVQPGGSMKLSCFVSGFTFSNSWMNWVRQSPEKGLEWVAQI
QLKSDNYATRYVESVRGRFIISRDDSKSSVSLQMNNLRTEDTGIYYCSSGTDY
WGQGTTLTVSS
Nucleic acid sequence (SEQ ID NO: 51):
GAAGTGAAGCTTGAGGAGTCTGGAGGAGGCTTGGTGCAACCTGGAGGAT
CCATGAAACTCTCCTGTTTTGTCTCTGGATTCACTTTCAGTAACTCCTGGA
TGAACTGGGTCCGCCAGTCTCCAGAGAAGGGGCTTGAGTGGGTTGCTCAG
ATTCAATTGAAATCTGATAATTATGCAACACGTTATGTGGAGTCTGTGAG
AGGGAGGTTCATCATCTCAAGAGATGATTCCAAAAGTAGTGTCTCCCTGC
AAATGAACAACTTAAGGACTGAAGACACTGGAATTTATTACTGTTCCTCT
GGGACCGACTACTGGGGCCAAGGCACCACTCTCACAGTCTCCTCA
33E7E12-VL
Amino acid sequence (SEQ ID NO: 52):
DVVMTQTPLALSVIIGQPASISCKSSQSLLFSNGKTYLNWLLQRPGQSPKRLIY
LVSNLDSGVPDRFTGSGSGTDFTLKIGRVEAEDLGVYYCVQGTHFPYTFGGG
TKLEIK
Nucleic acid sequence (SEQ ID NO: 53):
GATGTTGTGATGACCCAGACTCCACTCGCTTTGTCGGTTATCATTGGACA
ACCAGCCTCTATCTCTTGCAAGTCAAGTCAGAGCCTCTTATTTAGTAATG
GAAAAACCTATTTGAATTGGTTATTACAGAGGCCAGGCCAGTCTCCAAAG
CGCCTAATCTATCTGGTGTCTAATCTGGACTCTGGAGTCCCTGACAGGTT
CACTGGCAGTGGATCAGGAACAGATTTTACACTGAAAATCGGCAGAGTGG
AGGCTGAAGATTTGGGAGTTTATTACTGCGTGCAAGGTACACATTTTCCA
TACACGTTCGGTGGGGGGACCAAGTTGGAAATAAAA
71A4F12-VH
Amino acid sequence (SEQ ID NO: 54):
EVQLQQSGPELVKPGASVKIPCKASGYTFTAYNMDWVKQSHGMSLEWIGDI
NPNNGDTVYNQNFKGKATLTVDKSSSTAYMELRSLTSEDTAVYYCARRGSY
YYGSSHYALDFWGQGTSVSVSS
Nucleic acid sequence (SEQ ID NO: 55):
GAGGTCCAGCTGCAACAGTCTGGACCTGAGCTGGTGAAGCCTGGGGCTTC
AGTGAAGATACCCTGCAAGGCTTCTGGATACACATTCACTGCCTACAACA
TGGACTGGGTGAAGCAGAGCCATGGAATGAGCCTTGAGTGGATTGGAGAT
ATTAATCCTAACAATGGTGATACTGTCTACAACCAGAATTTCAAGGGCAA
GGCCACATTGACTGTAGACAAGTCCTCCAGCACAGCCTACATGGAACTCC
GCAGCCTGACATCTGAGGACACTGCAGTCTATTACTGTGCAAGAAGGGGG
TCCTATTACTACGGTAGTAGTCACTATGCTCTGGACTTCTGGGGTCAAGG
AACCTCAGTCTCCGTCTCCTCA
71A4F12-VL
Amino acid sequence (SEQ ID NO: 56):
DIQMTQITSSLSASLGNRVTISCRASQDIRNYLNWYQQKPDGTVKLLIYYTSR
LLSGVPSRFSGSGSGTDYSLTISNLEQEDIATYFCQQDNSLPRTFGGGTRLEIR
Nucleic acid sequence (SEQ ID NO: 57):
GATATCCAGATGACACAGATTACATCCTCCCTGTCTGCCTCTCTGGGAAA
CAGAGTCACCATCAGTTGCAGGGCAAGTCAGGACATTAGGAATTATTTAA
ACTGGTATCAGCAGAAACCAGATGGAACTGTTAAACTCCTGATCTACTAC
ACATCAAGATTACTCTCAGGAGTCCCATCACGGTTCAGTGGCAGTGGGTC
TGGAACAGATTATTCTCTCACCATTAGCAACCTGGAGCAAGAAGATATTG
CCACTTACTTTTGCCAACAGGATAATTCGCTTCCTCGGACGTTCGGTGGA
GGCACCAGGCTGGAAATCAGA
56A7E3-VH
Amino acid sequence (SEQ ID NO: 58):
EVQLQQSGPELVKPGASVKIPCKASGYTFTAYNMDWVKQSHGMSLEWIGDI
NPINGGTIYNQNFEGKATLTVDKSSSTAYLELRSLTSEDTAVYYCARRGSYYY
GSSHYALDFWGQGTSVSVSS
Nucleic acid sequence (SEQ ID NO: 59):
GAGGTCCAGCTGCAACAGTCTGGACCTGAGCTGGTGAAGCCTGGGGCTTC
AGTGAAGATACCCTGCAAGGCTTCTGGATACACATTCACTGCCTACAACA
TGGACTGGGTGAAGCAGAGCCATGGAATGAGCCTTGAGTGGATTGGAGAT
ATTAATCCTATCAATGGTGGTACTATCTACAACCAGAATTTCGAGGGCAA
GGCCACATTGACTGTAGACAAGTCTTCCAGCACAGCCTACTTGGAACTCC
GCAGCCTGACATCTGAGGACACTGCAGTCTATTACTGTGCAAGAAGGGGG
TCCTATTACTACGGTAGTAGCCACTATGCTTTGGACTTCTGGGGTCAAGG
AACCTCAGTCTCCGTCTCCTCA
56A7E3-VL
Amino acid sequence (SEQ ID NO: 64):
DIQMTQITSSLSASLGDRVTITCRASQDISNYLNWYQQKPDGTVKLLIYYTSRL
LSGVPSRFSGSGSGTDYSLTISNLEQEDIATYFCQQDHTLPRTFGGGTKLEIR
Nucleic acid sequence (SEQ ID NO: 61):
GATATCCAGATGACACAGATTACATCCTCCCTGTCTGCCTCTCTGGGAGA
CAGAGTCACCATCACTTGCAGGGCAAGTCAGGACATTAGCAATTATTTAA
ACTGGTATCAGCAGAAACCAGATGGAACTGTTAAACTCCTGATCTACTAC
ACATCAAGATTACTCTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGATCATACGCTTCCTCGGACGTTCGGTGGA
GGCACCAAGCTGGAAATCAGA
58F10-1F6-VH
Amino acid sequence (SEQ ID NO: 62):
EVQLRQSGPELVKPGAAVKISCKASGYSFTGYHMNWVKQSPEKSLEWIGEIN
PITSGITYNQIFKAKATLTVDKSSSTAYLQLKSLTSEDSAVYYCTRRDYGTSLD
YWGQGTTLTVSS
Nucleic acid sequence (SEQ ID NO: 63):
GAGGTCCAGCTGCGGCAGTCTGGACCTGAGCTGGTGAAGCCTGGGGCTGC
GGTGAAGATATCCTGCAAGGCTTCTGGTTACTCATTCACTGGCTACCACA
TGAACTGGGTGAAGCAAAGTCCTGAAAAGAGCCTTGAGTGGATTGGAGA
GATTAATCCTATCACTAGTGGTATTACCTACAACCAGATTTTCAAGGCCAA
GGCCACATTGACTGTAGACAAATCCTCCAGCACAGCCTACTTGCAGCTCA
AGAGCCTGACATCTGAGGACTCTGCAGTCTATTACTGTACAAGAAGGGAC
TACGGTACTAGCCTTGACTACTGGGGCCAAGGCACCACTCTCACAGTCTC
CTCA
58F10-1F6-VL
Amino acid sequence (SEQ ID NO: 64):
DIQMTQITSSLSASLGDRVTITCRASQDISNYLNWYQQKPDGTVKLLIYYTSRL
LSGVPSRFSGSGSGTDYSLTISNLEQEDIATYFCQQDHTLPRTFGGGTKLEIR
Nucleic acid sequence (SEQ ID NO: 65):
GATATCCAGATGACACAGATTACATCCTCCCTGTCTGCCTCTCTGGGAGA
CAGAGTCACCATCACTTGCAGGGCAAGTCAGGACATTAGCAATTATTTAA
ACTGGTATCAGCAGAAACCAGATGGAACTGTTAAACTCCTGATCTACTAC
ACATCAAGATTACTCTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGATCATACGCTTCCTCGGACGTTCGGTGGA
GGCACCAAGCTGGAAATCAGA
73H5-1G2-VH
Amino acid sequence (SEQ ID NO: 66):
QVQLKQSGPGLVQPSQSLSITCTVSGFSLTNYGVHWIRQSPGKGLEWLGVIW
RGGSTDYNAAFMSRLSITKDNSKSQVFFQMNSLQAADTAIYYCANNYGYDC
FAYWGQGTLVTVSA
Nucleic acid sequence (SEQ ID NO: 67):
CAGGTGCAGCTGAAGCAGTCAGGACCTGGCCTAGTGCAGCCCTCACAGAG
CCTGTCCATAACCTGCACAGTCTCTGGTTTCTCATTAACTAACTATGGTG
TACACTGGATTCGCCAGTCTCCAGGAAAGGGTCTGGAGTGGCTGGGAGTG
ATATGGAGAGGTGGAAGCACAGACTACAATGCAGCTTTCATGTCCAGACT
GAGCATCACCAAGGACAACTCCAAGAGCCAAGTTTTCTTTCAAATGAACA
GTCTGCAAGCTGCTGACACTGCCATATATTACTGTGCCAACAACTATGGT
TACGACTGCTTTGCTTACTGGGGCCAAGGGACTCTGGTCACTGTCTCTGC
A
73H5-1G2-VL
Amino acid sequence (SEQ ID NO: 68):
DVQITQSPSYLAASPGETITINCRASKSIIKYLAWYQEKPGKTNKLLIYSGSTLQ
SGIPSRFSGSGSGTDFTLTITSLEPEDFAMYYCQQHNEYPLTFGAGTRLELK
Nucleic acid sequence (SEQ ID NO: 69):
GATGTCCAAATAACCCAGTCTCCATCTTATCTTGCTGCATCTCCTGGAGA
AACCATTACTATTAATTGCAGGGCAAGTAAGAGTATTATCAAATATTTAG
CCTGGTATCAAGAGAAACCTGGGAAAACTAATAAGCTTCTTATCTACTCT
GGATCCACTTTGCAATCTGGAATTCCATCAAGGTTCAGTGGCAGTGGATC
TGGTACAGATTTCACTCTCACCATCACTAGCCTGGAGCCTGAAGATTTTG
CAATGTATTACTGTCAACAGCATAATGAATACCCGCTCACGTTCGGTGCT
GGGACCAGGCTGGAGCTGAAA
110D4G4-VH
Amino acid sequence (SEQ ID NO: 70):
EVQLQQSGPGLVKPGASVKISCRASGYLFTGYYMNWVKQSPEKSLEWIGEIN
PNTGDPRYDQKFKAKATLTVDRSSSTAYMHLKSLTSEDSAVYFCARRDYGTS
LDYWGQGTTLTVTS
Nucleic acid sequence (SEQ ID NO: 71):
GAGGTTCAGCTGCAGCAGTCTGGACCTGGGCTGGTGAAGCCTGGGGCTTC
AGTGAAGATATCCTGCAGGGCTTCTGGTTACCTATTCACTGGCTACTACA
TGAACTGGGTGAAGCAAAGTCCTGAAAAGAGCCTTGAGTGGATTGGAGA
GATTAATCCTAACACTGGTGATCCTAGATACGACCAGAAGTTTAAGGCCA
AGGCCACATTGACTGTAGACAGATCCTCCAGCACAGCCTACATGCACCTC
AAGAGCCTGACATCTGAAGACTCTGCAGTCTATTTCTGTGCCAGGAGGGA
CTACGGTACTAGCCTTGACTACTGGGGCCAAGGCACCACTCTCACAGTCA
CCTCA
110D4G4-VL
Amino acid sequence (SEQ ID) NO: 72):
DVQITQSPSYLAASPGETITINCRASKSISKYLAWYQEKPGKTNKLLIYSGSTL
QSGIPSRFSGSGSGSDFTLTISSLEPEDFAMYYCQQHNEYPLTFGAGTKLELK
Nucleic acid sequence (SEQ ID NO: 73):
GATGTCCAGATAACCCAGTCTCCATCTTATCTTGCTGCATCTCCTGGAGA
AACCATTACTATTAATTGCAGGGCAAGTAAGAGCATTAGCAAATATTTAG
CCTGGTATCAAGAGAAACCTGGGAAAACTAATAAGCTTCTTATCTACTCT
GGATCCACTTTGCAATCTGGAATTCCATCAAGGTTCAGTGGCAGTGGATC
TGGTTCAGATTTCACTCTCACCATCAGTAGCCTGGAGCCTGAAGATTTTG
CAATGTATTACTGTCAACAGCATAATGAATATCCGCTCACGTTCGGTGCT
GGGACCAAGCTGGAGCTGAAA
81A1F8-VH
Amino acid sequence (SEQ ID NO: 74):
EVQLQQSGPELVKPGASVKISCKATGYLFTGYYMNWVKQSPEKSLEWIGEIN
PNTGDTNNNQKFKAKATMTVDRSSSTAYMQLKRLTSEDSAVYYCVRRDYG
TSLDYWGQGTTLTVSS
Nucleic acid sequence (SEQ ID NO: 75):
GAGGTTCAGCTGCAGCAGTCTGGACCTGAGTTGGTGAAGCCTGGGGCTTC
AGTGAAGATATCCTGCAAGGCTACTGGTTACTTATTCACTGGCTACTACA
TGAACTGGGTGAAACAAAGTCCTGAAAAGAGCCTTGAGTGGATTGGAGA
GATCAATCCTAACACTGGTGATACTAACAACAACCAGAAGTTCAAGGCCA
AGGCCACAATGACTGTTGACAGATCCTCCAGCACAGCCTACATGCAGCTC
AAGAGGCTGACATCTGAGGACTCTGCAGTCTATTATTGTGTAAGAAGGGA
CTACGGTACTAGTCTTGACTACTGGGGCCAAGGCACCACTCTCACAGTCTC
CTCA
81A1F8-VL
Amino acid sequence (SEQ ID NO: 76):
DVQITQSPSYLAASPGETITINCRASKSINKYLAWYQEKPGKTNKLLIHSGSTL
QSGIPSRFSGSRSGTDFTLTISNLEPEDFAMYYCQQHNEYPITFGAGTKLELK
Nucleic acid sequence (SEQ ID NO: 77):
GATGTCCAGATAACCCAGTCTCCATCTTATCTTGCTGCATCTCCTGGAGA
AACCATTACTATTAATTGCAGGGCAAGTAAGAGTATTAACAAATATTTAG
CCTGGTATCAAGAGAAACCTGGGAAAACTAATAAGCTTCTTATCCACTCT
GGATCCACTTTGCAATCTGGAATTCCATCAAGGTTCAGTGGCAGTAGATC
TGGAACAGATTTCACTCTCACCATCAGTAACCTGGAGCCTGAAGATTTTG
CAATGTATTACTGTCAACAACATAATGAATATCCGATCACGTTCGGTGCT
GGGACCAAGCTGGAGCTGAAA
100F2E3-VH
Amino acid sequence (SEQ ID NO: 78):
QVQLQQPGAELVRPGSSVKLSCKTSDYTFTSYWMDWVKQRPGQGLEWIGNI
YPSDSKTHSNQKFRDKATLTVDKSSSTAYMQLNSLTSEDSAVYYCARRGGY
YGYAMDYWGQGTSVTVSS
Nucleic acid sequence (SEQ ID NO: 79):
CAGGTCCAACTGCAGCAGCCTGGGGCTGAACTGGTGAGGCCTGGGTCTTC
AGTGAAGCTGTCCTGCAAGACTTCTGACTACACCTTCACCAGCTACTGGA
TGGATTGGGTGAAGCAGAGGCCTGGACAAGGCCTTGAATGGATTGGTAAC
ATTTACCCTTCTGATAGTAAAACTCACTCCAATCAAAAGTTCAGGGACAA
GGCCACATTGACTGTAGACAAATCCTCCAGCACAGCCTACATGCAGCTCA
ACAGCCTGACATCTGAAGACTCTGCGGTCTATTACTGTGCAAGAAGAGGT
GGTTACTACGGCTATGCTATGGACTACTGGGGTCAAGGAACCTCAGTCAC
CGTCTCCTCA
100F2E3-VL
Amino acid sequence (SEQ ID NO: 80):
DIVMTQSPSSLSVSAGEKVTMSCKSSQSLLNSGNQDNYLAWYQQKPGQPPKL
LIYGASIRESGVPDRFTGSGSGTDFTLTISSVQAEDLAVYYCQNDHSYPYTFGG
GTKLEIK
Nucleic acid sequence (SEQ ID NO: 81):
GACATTGTGATGACACAGTCTCCATCCTCCCTGAGTGTGTCAGCAGGAGA
GAAGGTCACTATGAGCTGCAAGTCCAGTCAGAGTCTGTTAAATAGTGGAA
ATCAAGACAACTACTTGGCCTGGTACCAGCAGAAACCAGGGCAGCCTCCT
AAACTGTTGATCTACGGGGCATCCATTAGGGAATCTGGGGTCCCTGATCG
CTTCACAGGCAGTGGATCTGGAACCGATTTCACTCTTACCATCAGTAGTG
TGCAGGCTGAAGACCTGGCAGTTTATTACTGTCAAAATGATCATAGTTAT
CCGTACACGTTCGGAGGGGGGACCAAGTTGGAAATAAAA
111F3A2-VH
Amino acid sequence (SEQ ID NO: 82):
QVQLKESGPGLVAPSQSLSITCTVSGFSLITYGVSWVRQPPGKGLEWLGVIWG
DGTTNYHSALKPRLTISKDDSKSQVLLKLNSLQTDDTATYYCANYYGYPFFA
YWGQGTLVTVSA
Nucleic acid sequence (SEQ ID NO: 83):
CAGGTGCAGCTGAAGGAGTCAGGACCTGGCCTGGTGGCGCCCTCACAGAG
CCTGTCCATCACATGCACTGTCTCAGGATTCTCATTAATCACCTATGGTG
TAAGCTGGGTTCGCCAGCCTCCAGGAAAGGGTCTGGAGTGGCTGGGAGTA
ATATGGGGTGACGGGACCACAAATTATCATTCAGCTCTCAAACCCAGACT
GACCATCAGCAAGGATGACTCCAAGAGCCAAGTCCTCTTAAAATTGAACA
GTCTGCAAACTGATGACACAGCCACGTACTACTGTGCCAACTACTATGGT
TACCCCTTTTTTGCTTACTGGGGCCAAGGGACTCTGGTCACTGTCTCTGC
A
111F3A2-VL
Amino acid sequence (SEQ ID NO: 84):
DIQMTQSSSSFSVSLGDRVTITCKASEDIYNRLAWYQQKPGSAPGVLISGATN
LVTGIPSRFSGSGSGKDYTLTITSLQTEDVATYYCQQYWSSPPTFGGGTKLEIK
Nucleic acid sequence (SEQ ID NO: 85):
GACATCCAGATGACACAATCTTCATCCTCCTTTTCTGTATCTCTAGGAGAC
AGAGTCACCATTACTTGCAAGGCAAGTGAGGACATATATAATCGGTTAGC
CTGGTATCAACAGAAACCAGGAAGTGCTCCTGGGGTCTTAATATCTGGTG
CAACCAATTTGGTAACTGGGATTCCTTCAAGATTCAGTGGCAGTGGATCT
GGAAAGGATTACACTCTCACCATTACCAGTCTTCAGACTGAAGATGTTGC
TACTTATTACTGTCAACAGTATTGGAGTAGTCCTCCGACGTTCGGTGGAGG
CACCAAGCTGGAAATCAAA
81A7A10-VH
Amino acid sequence (SEQ ID NO: 86):
QVQLKESGPGLVAPLQSLSITCTVSGFSLSSYGVSWVRQLPGKGLEWLGVIW
GDGSTDYHSTLTSRLSFSKDNSESQVFLKLNSLQTDDTATYYCAIFYGYDEGF
AYWGQGTLVTVSA
Nucleic acid sequence (SEQ ID NO: 87):
CAGGTGCAGCTGAAGGAGTCAGGACCTGGCCTGGTGGCGCCCTTACAGAG
CCTGTCCATCACATGCACTGTCTCAGGGTTCTCATTAAGCAGTTATGGTGT
AAGCTGGGTTCGCCAGCTTCCAGGAAAGGGTCTGGAGTGGCTGGGAGTAA
TATGGGGTGACGGGAGCACAGATTATCATTCAACTCTCACATCCAGACTG
AGCTTCAGCAAGGATAATTCCGAGAGCCAAGTTTTCTTAAAACTGAATAG
TCTGCAAACTGATGACACAGCCACATACTACTGTGCCATTTTCTATGGTTA
CGACGAAGGGTTTGCTTATTGGGGCCAAGGGACTCTGGTCACTGTCTCTG
CA
81A7A10-VL
Amino acid sequence (SEQ ID NO: 88):
ENVLTQSPAIMSAFLGEKVTMSCRASSTVNYMYWYQQKSDASPKVWIYYTS
RLAPGVPARFSGSGSGNSFSLTISSMEGEDAATYYCQQFTSSPLTFGAGTKLEL
R
Nucleic acid sequence (SEQ ID NO: 89):
GAAAATGTGCTCACCCAGTCTCCAGCAATCATGTCTGCATTTCTAGGGGA
GAAGGTCACCATGAGCTGCAGGGCCAGCTCAACTGTAAATTACATGTACT
GGTACCAGCAGAAGTCAGATGCCTCCCCCAAAGTTTGGATTTATTACACA
TCCAGGTTGGCTCCTGGAGTCCCAGCTCGCTTCAGTGGCAGTGGGTCTGG
GAACTCTTTTTCTCTCACAATCAGCAGCATGGAGGGTGAAGATGCTGCCA
CTTATTACTGCCAGCAGTTTACTAGTTCCCCGCTCACGTTCGGTGCTGGGA
CCAAGCTGGAGCTGAGA
107D10D11-VH
Amino acid sequence (SEQ ID NO: 101):
QIQLKQSGPGLVQPSQSLSITCTVSGFSLSSYGVNWVRQSPGKGLEWLGMIWS
GGNTDYNAAFISRVSFTKDNSKSQVFFTMNSLQADDTAIYYCARIWYYGMD
YWGQGTSVTVSS
Nucleic acid sequence (SEQ ID NO: 102):
CAGATACAGCTGAAGCAGTCAGGACCTGGCCTAGTGCAGCCCTCACAGAG
TCTGTCCATCACCTGCACAGTCTCTGGTTTCTCATTAAGTAGTTATGGTGT
AAATTGGGTTCGCCAGTCTCCAGGAAAGGGTCTGGAATGGCTGGGAATGA
TATGGAGTGGTGGAAACACAGACTATAATGCAGCTTTCATATCCAGAGTG
AGCTTCACCAAGGACAATTCCAAGAGCCAAGTTTTCTTTACAATGAACAG
TCTGCAAGCTGATGACACAGCCATATATTACTGTGCCAGAATATGGTACT
ATGGTATGGACTACTGGGGTCAGGGAACCTCAGTCACCGTCTCCTCA
107D10D11-VL
Amino acid sequence (SEQ ID NO: 103):
DIVMTQSQKFMSTTVGDRVSITCKASQNVGTAVAWFQQKPGQSPKILIYSASI
RYPGVPDRFTGSGSGTDFTLTTSNVQSEDLADYFCLQYITFPRTFGGGTKLYIK
Nucleic acid sequence (SEQ ID NO: 110):
GACATTGTGATGACCCAGTCTCAAAAATTCATGTCCACAACAGTAGGAGA
CAGGGTCAGCATCACCTGCAAGGCCAGTCAGAATGTGGGTACTGCTGTAG
CCTGGTTTCAACAGAAACCAGGACAATCTCCTAAAATACTGATTTATTCA
GCATCCATTCGCTACCCTGGAGTCCCTGATCGCTTCACAGGCAGTGGATCT
GGGACAGATTTCACTCTCACCACTAGCAATGTGCAGTCTGAAGACCTGGC
AGATTATTTCTGTCTGCAATATATCACCTTTCCTCGGACGTTCGGTGGAGG
CACCAAGCTGTATATCAAA
101H9G9A2-VH
Amino acid sequence (SEQ ID NO: 109):
QVQLLQPGAELVRPGSSVKLSCKASDYTFTSYWMDWVKQRPGQGLEWIGNI
YPSDSKTHYNQKFKDKATLTVDKSSSTAYMQLNSLTSEDSAVYYCARRGGY
YAYAMDYWGQGTSVTVSS
Nucleic acid sequence (SEQ ID NO: 204):
CAGGTGCAGCTGCTGCAGCCCGGCGCTGAGCTGGTGAGACCCGGAAGCA
GCGTGAAGCTGAGCTGCAAAGCCAGCGACTACACATTCACAAGCTACTGG
ATGGACTGGGTGAAACAGAGACCTGGCCAGGGACTGGAATGGATTGGAA
ACATCTACCCCAGTGACAGCAAGACCCACTACAACCAGAAGTTCAAGGAC
AAAGCCACCCTGACCGTGGACAAGAGCAGCAGCACCGCCTACATGCAGCT
GAACAGCCTGACCAGCGAAGACAGCGCCGTGTACTACTGCGCCAGAAGA
GGCGGCTACTACGCCTATGCCATGGACTACTGGGGGCAGGGAACCAGCGT
GACCGTGAGCAGC
101H9G9A2-VL
Amino acid sequence (SEQ ID NO: 111):
DIVMTQSPSSLSVSAGEKVTMRCKSSQSLLNSGNQDNYLAWYQQKPGQPPKL
LLYGARIRESGVPDRFIGSGSGTDFTLTISSVQAEDLAVYYCQNDHSYPYTFG
GGTKVEIK
Nucleic acid sequence (SEQ ID NO: 205):
GACATTGTGATGACCCAGAGCCCCAGCAGCCTGAGCGTGAGCGCCGGAG
AAAAAGTGACCATGAGATGCAAGAGCAGCCAGAGCCTGCTGAACAGCGG
GAACCAGGACAACTACCTGGCCTGGTATCAACAGAAGCCCGGCCAGCCCC
CCAAACTGCTGCTGTATGGAGCCAGAATCAGAGAGAGCGGCGTGCCCGAC
AGATTCATCGGCAGCGGCAGCGGAACCGACTTCACCCTGACCATCAGCAG
CGTGCAGGCTGAGGACCTGGCCGTGTACTACTGCCAGAACGACCACAGCT
ACCCCTATACCTTTGGAGGCGGCACCAAGGTGGAGATCAAA
173B1C1-VH
Amino acid sequence (SEQ ID NO: 118):
QVQLQQSGAELARPGASVKMSCKASGYTFTSNTMHWVKQRPGQGLEWIGYI
NPGSAYTNYNQKFNDKATLTADKSSSTASMQLSSLTSEDSAVYYCARLATYY
DNDGYAMDYWGQGTSVTVSS
Nucleic acid sequence (SEQ ID NO: 206):
CAGGTCCAGCTGCAGCAGTCTGGGGCTGAACTGGCAAGACCTGGTGCCTC
AGTGAAGATGTCCTGCAAGGCTTCTGGCTACACCTTTACTAGCAACACGA
TGCACTGGGTAAAACAGAGGCCTGGACAGGGTCTGGAATGGATTGGATAC
ATTAATCCTGGCAGTGCTTATACTAACTACAATCAGAAGTTCAATGACAA
GGCCACATTGACTGCAGACAAGTCCTCCAGCACAGCCTCCATGCAACTGA
GCAGCCTGACATCTGAGGACTCTGCAGTCTATTACTGTGCAAGACTCGCG
ACCTACTATGATAACGACGGATATGCTATGGACTATTGGGGTCAAGGAAC
CTCAGTCACCGTCTCCTCA
173B1C1-VL(1)
Amino acid sequence (SEQ ID NO: 120):
DIQMTQSTSSLSASLGDRVTISCRASQDISNYLNWYQQKPDGTVKLLIYYTSK
LHSGVPSRFSGSGSGTDFSLTITNLEQEDFATYFCQQGHTLPWTFGGGTKLEIK
Nucleic acid sequence (SEQ ID NO: 207):
GACATCCAGATGACACAGTCTACATCCTCCCTGTCTGCCTCTCTGGGAGAC
AGAGTCACCATCAGTTGCAGGGCAAGTCAGGACATTAGTAATTATTTAAA
CTGGTATCAGCAGAAACCAGATGGAACTGTTAAACTCCTGATCTACTACA
CATCAAAATTACACTCAGGAGTCCCATCAAGGTTCAGTGGCAGTGGGTCT
GGAACAGATTTTTCTCTCACCATTACCAACCTGGAGCAAGAAGATTTTGCC
ACTTACTTTTGCCAACAGGGTCATACGCTTCCGTGGACGTTCGGTGGAGGC
ACCAAACTGGAGATCAAA
173B1C1-VL(2)
Amino acid sequence (SEQ ID NO: 221):
DIQMTQTTSSLSASLGDRVTISCRASQDISNYLNWYQQKPDGTVKLLIYYTSK
LHSGVPSRFSGSGSGTDFSLTITNLEQEDFATYFCQQGHTLPWTFGGGTKLEIK
Nucleic acid sequence (SEQ ID NO: 208):
GATATCCAGATGACACAGACTACATCCTCCCTGTCTGCCTCTCTGGGAGAC
AGAGTCACCATCAGTTGCAGGGCAAGTCAGGACATTAGTAATTATTTAAA
CTGGTATCAGCAGAAACCAGATGGAACTGTTAAACTCCTGATCTACTACA
CATCAAAATTACACTCAGGAGTCCCATCAAGGTTCAGTGGCAGTGGGTCT
GGAACAGATTTTTCTCTCACCATTACCAACCTGGAGCAAGAAGATTTTGCC
ACTTACTTTTGCCAACAGGGTCATACGCTTCCGTGGACGTTCGGTGGAGGC
ACCAAACTGGAGATCAAA
107D10E11-VH
Amino acid sequence (SEQ ID NO: 122):
EVQLQQSGTGLVKPGTSVKLSCKASGYSFTSYWITWVKQRPGQGLEWIGDIS
PAGGGRNYNERFKNKATLTVDASSSTAYMQLSSLTSEDSAVYYCVRGDSTV
DLDYWGQGTTLTVSS
Nucleic acid sequence (SEQ ID NO: 228):
GAGGTTCAGCTGCAGCAGTCTGGGACTGGACTTGTGAAGCCTGGGACTTC
AGTGAAGTTGTCCTGCAAGGCTTCTGGCTACAGCTTCACCAGCTACTGGAT
AACCTGGGTGAAACAGAGGCCTGGACAAGGCCTTGAGTGGATTGGAGAT
ATTTCTCCTGCTGGTGGTGGTCGTAACTACAATGAGAGATTCAAGAACAA
GGCCACACTGACTGTAGACGCATCCTCCAGCACAGCCTACATGCAGCTCA
GCAGCCTGACATCTGAGGACTCTGCGGTCTATTACTGTGTAAGAGGTGAT
AGTACGGTAGACTTAGACTACTGGGGCCAAGGCACCACTCTCACAGTCTC
CTCA
107D10E11-VL
Amino acid sequence (SEQ ID NO: 124):
DIVMTQAAPSVPVTPGESVSISCRSSKSLLHSNGNTYLYWFLQRPGQSPQLLIY
RVSNLASGVPDRFSGSGSGTAFTLRISRVEAEDVGVYYCMQHLEYPYTFGGG
TKLEIK
Nucleic acid sequence (SEQ ID NO: 229):
GATATTGTGATGACTCAGGCTGCACCCTCTGTACCTGTCACTCCTGGAGAG
TCAGTATCCATCTCCTGCAGGTCTAGTAAGAGTCTCCTGCATAGTAATGGC
AACACTTACTTGTATTGGTTCCTGCAGAGGCCAGGCCAGTCTCCTCAGCTC
CTGATATATCGGGTGTCCAACCTTGCCTCAGGAGTCCCAGACAGGTTCAG
TGGCAGTGGGTCAGGGACTGCTTTCACACTGAGAATCAGTAGAGTGGAGG
CTGAGGATGTGGGTGTTTATTACTGTATGCAACATCTAGAATATCCGTACA
CGTTCGGAGGGGGGACCAAGCTGGAAATAAAA
109G10A6-VH
Amino acid sequence (SEQ ID NO: 132):
EVLLQQSGPELVKPGASVKIPCKASGYGFTDYNMDWIKQSHGKSLEWIGDIN
PHNGDTIYNQKFKGKAKLTVDKSSTTAYMELRSLASEDTAVYYCARRGAYY
YGSSHYALDFWGQGTSVTVSS
Nucleic acid sequence (SEQ ID NO: 209):
GAGGTCCTGCTGCAACAGTCTGGACCTGAACTGGTGAAGCCTGGGGCTTC
AGTGAAGATACCCTGCAAGGCTTCTGGATACGGATTCACTGACTACAACA
TGGACTGGATAAAACAGAGCCATGGAAAGAGCCTTGAGTGGATTGGAGA
TATTAATCCTCACAATGGTGATACTATCTACAACCAGAAGTTCAAGGGCA
AGGCCAAATTGACTGTAGACAAGTCCTCCACCACTGCCTACATGGAACTC
CGCAGCCTGGCATCTGAGGACACCGCAGTCTATTACTGTGCAAGGAGGGG
GGCATATTACTACGGTAGTAGCCACTATGCTCTGGACTTCTGGGGTCAAG
GAACCTCAGTCACCGTCTCCTCA
109G10A6-VL
Amino acid sequence (SEQ ID NO: 134):
DIQMTQTTSSLSASLGDRVTISCRASQDITNYLNWYQQKPDGTVKLLIYYTSR
LFSGVPSRFSGSGSGTDYSLTISNLKREDIATYFCQQGNTLPWTFGGGTKLEIK
Nucleic acid sequence (SEQ ID NO: 210):
GATATCCAGATGACACAGACTACATCCTCCCTGTCTGCCTCTCTGGGAGAC
AGAGTCACCATCAGTTGCAGGGCAAGTCAGGACATTACCAATTATTTAAA
CTGGTATCAGCAGAAACCAGATGGAACTGTTAAACTCCTGATCTACTACA
CATCAAGATTATTCTCAGGAGTCCCATCAAGGTTCAGTGGCAGTGGGTCT
GGAACAGATTATTCTCTCACCATTAGCAACCTGAAACGAGAAGATATTGC
CACTTACTTTTGCCAACAGGGTAATACGCTTCCGTGGACGTTCGGTGGAG
GCACCAAGCTGGAAATCAAA
94G12D11-VH
Amino acid sequence (SEQ ID NO: 141):
EVQLQQSGPELVKPGASVKISCKASGYLFTGYYMNWVKQSPEKSLEWIGEIN
PNKGDSNLNQNFKAKATLTVDKSSSTAYMQFKSLTFEDSAVYFCVRRDYGTS
LDHWGQGTTLTVSS
Nucleic acid sequence (SEQ ID NO: 211):
GAGGTTCAGCTGCAGCAGTCTGGACCTGAGCTGGTGAAGCCTGGGGCTTC
AGTGAAGATATCCTGCAAGGCCTCTGGTTACTTATTCACTGGCTACTACAT
GAACTGGGTGAAACAAAGTCCTGAAAAGAGTCTTGAGTGGATTGGAGAG
ATTAATCCTAACAAAGGTGATAGTAATCTCAACCAGAATTTCAAGGCCAA
GGCCACATTGACTGTTGACAAATCCTCCAGCACAGCCTACATGCAGTTCA
AGAGCCTGACATTTGAGGACTCTGCAGTCTATTTCTGTGTAAGAAGGGAC
TACGGCACTAGTCTTGACCACTGGGGCCAAGGCACGACTCTCACAGTCTC
CTCA
94G12D11-VL
Amino acid sequence (SEQ ID NO: 143):
DVQITQSPSYLATSPGETITINCRASKSISKYLAWYQEKPGKTKKLLIYSGSTL
HTAIPSRFSGRGFGTDFTLTISSLEPEDFAMYYCQQHNEYPITFGGGTKLELK
Nucleic acid sequence (SEQ ID NO: 212):
GATGTCCAGATAACCCAGTCTCCATCTTATCTTGCTACATCTCCTGGAGAA
ACCATTACTATTAATTGCAGGGCAAGTAAGAGTATTAGCAAATATTTAGC
CTGGTATCAAGAGAAACCTGGGAAAACTAAGAAGCTTCTTATCTACTCTG
GATCCACTTTGCATACTGCAATTCCATCAAGGTTCAGTGGCCGTGGATTTG
GTACAGATTTCACTCTCACCATCAGTAGCCTGGAGCCTGAAGATTTTGCAA
TGTATTACTGTCAACAGCATAATGAATACCCAATCACATTCGGTGGTGGG
ACCAAGCTGGAGCTGAAA
92F5B8-VH
Amino acid sequence (SEQ ID NO: 146):
EVQLQQSGPELVKPGASVKISCKATGYLFTGYYMNWVKQSPEKSLEWIGEIN
PNTGDTNNNQKFKAKATMTVDRSSSTAYMQLKSLTSEDSAVYYCVRRDYGT
SLDYWGQGTTLTVSS
Nucleic acid sequence (SEQ ID NO: 213):
GAGGTCCAGCTGCAGCAGTCTGGACCTGAGTTGGTGAAGCCTGGGGCTTC
AGTGAAGATATCCTGCAAGGCTACTGGTTACTTATTCACTGGCTACTACAT
GAACTGGGTGAAGCAAAGTCCTGAAAAGAGCCTTGAGTGGATTGGAGAG
ATCAATCCTAACACTGGTGATACTAACAACAACCAGAAGTTCAAGGCCAA
GGCCACAATGACTGTTGACAGATCCTCCAGTACAGCCTACATGCAGCTCA
AGAGCCTGACATCTGAGGACTCTGCAGTCTATTATTGTGTAAGAAGGGAC
TACGGTACTAGCCTTGACTACTGGGGCCAAGGCACCACTCTCACAGTCTC
CTCA
92F5B8-VL
Amino acid sequence (SEQ ID NO: 148):
DVQITQSPSYLAASPGETITINCRASKSINKYLAWYQEKPGKTNKLLIHSGSTL
HSGIPSRFSGSRSGTDFTLTISNLEPEDFAMYYCQQHNEYPLTFGAGTKLELK
Nucleic acid sequence (SEQ ID NO: 214):
GATGTCCAGATAACCCAGTCTCCATCTTATCTTGCTGCATCTCCTGGAGAA
ACCATTACTATTAATTGCAGGGCAAGTAAGAGTATTAACAAATATTTAGC
CTGGTATCAAGAGAAACCTGGGAAAACTAATAAGCTTCTTATCCACTCTG
GATCCACTTTGCACTCTGGAATTCCATCAAGGTTCAGTGGCAGTAGATCTG
GAACAGATTTCACTCTCACCATCAGTAACCTGGAGCCTGAAGATTTTGCA
ATGTATTACTGTCAACAACATAATGAATACCCGCTCACGTTCGGTGCTGG
GACCAAGCTGGAGCTGAAA
185B11E3-VH
Amino acid sequence (SEQ ID NO: 156):
QVQLQQSGAELARPGASVKMSCKASGYTFTSNTMHWVKQRPGQGLEWIGYI
NPGSAYTNYNQKENDKATLTADKSSSTASMQLSSLTSEDSAVYYCARLATYY
DNDGYAMDYWGQGTSVTVSS
Nucleic acid sequence (SEQ ID NO: 215):
CAGGTGCAGCTGCAGCAGAGCGGCGCCGAGCTGGCTAGACCCGGAGCTTC
CGTGAAGATGAGCTGCAAAGCCAGCGGCTACACATTCACATCCAACACAA
TGCACTGGGTGAAACAGAGGCCTGGCCAGGGACTGGAGTGGATTGGGTA
CATCAACCCCGGCAGCGCCTACACCAACTACAACCAGAAATTCAATGACA
AAGCCACACTGACCGCCGACAAAAGCAGCAGCACAGCCAGCATGCAGCT
GAGCAGCCTGACCTCCGAGGACAGCGCTGTGTACTACTGCGCCAGACTGG
CCACCTACTACGACAACGACGGATACGCCATGGATTACTGGGGACAGGGA
ACCAGCGTGACCGTGAGCAGC
185B11E3-VL
Amino acid sequence (SEQ ID NO: 158):
ENVLTQSPAIMSASPGEKVTMTCSASSSVNYMYWYQQRSSTSPKLWIYGTSK
LASGVPGRFSGSGSGNSYSLTISSMEAEDVATYYCFQGSGYPYTFGGGTKLEI
K
Nucleic acid sequence (SEQ ID NO: 216):
GAGAATGTGCTGACCCAGAGCCCCGCCATCATGAGTGCCAGCCCAGGAGA
GAAGGTGACAATGACATGCAGCGCCAGCAGCAGCGTGAATTACATGTACT
GGTACCAGCAGAGAAGCAGCACCAGCCCCAAGCTGTGGATCTACGGCAC
CAGCAAGCTGGCCAGCGGAGTGCCCGGCAGATTCTCCGGAAGTGGAAGC
GGAAATAGCTACAGCCTGACTATCAGCAGCATGGAGGCCGAAGATGTTGC
TACCTACTACTGCTTTCAGGGCAGCGGCTACCCCTACACCTTCGGCGGCGG
CACCAAGCTGGAGATCAAG
168B11E2-VH
Amino acid sequence (SEQ ID NO: 164):
QVQLKESGPGLVAPSQSLSITCTVSGFSLTSYGVSWVRQPPGKGLEWLGVIW
GDGNTNYHSALISRLSISKDNSKSQVFLKLNSLQTDDTATYYCANLYVLFAY
WGQGTLVTVSA
Nucleic acid sequence (SEQ ID NO: 217):
CAGGTGCAGCTGAAGGAAAGCGGCCCCGGCCTGGTGGCCCCATCTCAGAG
TCTGAGCATCACCTGCACAGTGAGCGGCTTCAGCCTGACAAGCTACGGCG
TGAGCTGGGTGAGACAGCCCCCCGGAAAGGGCCTGGAATGGCTGGGCGT
GATTTGGGGCGACGGCAACACAAACTACCACAGCGCCCTGATCAGCAGAC
TGAGCATCAGCAAGGACAATAGCAAGAGCCAGGTGTTCCTGAAGCTGAA
CAGCCTGCAGACCGACGACACCGCCACCTACTACTGCGCCAACCTGTACG
TGCTGTTCGCCTACTGGGGCCAGGGCACCCTGGTGACCGTGAGCGCT
168B11E2-VL
Amino acid sequence (SEQ ID NO: 166):
ENVLTQSPAIMSASPGEKVTMTCSASSSVSYMHWYQQKSSTSPKLWIYGTSK
LASGVPGRFSGSGSGNSYSLTISSMEAEDVATYYCFQGSGYPYTFGGGTKLEI
K
Nucleic acid sequence (SEQ ID NO: 218):
GAGAATGTGCTGACCCAGAGCCCCGCCATCATGAGTGCCAGCCCAGGAGA
GAAGGTGACAATGACATGCAGCGCCAGCAGCAGCGTGAGTTACATGCACT
GGTACCAGCAGAAGAGCAGCACCAGCCCCAAACTGTGGATCTACGGGAC
CAGCAAGCTGGCCAGCGGAGTGCCCGGAAGATTCAGTGGCAGCGGAAGC
GGCAATAGCTACAGCCTGACTATCAGCAGCATGGAGGCCGAAGATGTTGC
TACCTACTACTGTTTTCAGGGAAGCGGCTACCCATACACCTTCGGGGGGG
GGACCAAGCTGGAAATCAAG
188H6D3-VH
Amino acid sequence (SEQ ID NO: 174):
QVQLQQPGTELVKPGASVKLSCKASGYTFTNYWIHWVKQRPGQGLEWIGDI
NPTNGGTNYNEKFKTKAILTVDRSSSTAYMQVSSLTSEDSAVYYCARSGGYD
FDYWGQGTTLTVSS
Nucleic acid sequence (SEQ ID NO: 219):
CAGGTGCAGCTGCAGCAGCCAGGCACAGAGCTGGTGAAGCCCGGCGCCA
GCGTGAAACTGAGCTGCAAAGCCAGCGGCTACACCTTCACAAACTACTGG
ATTCATTGGGTGAAGCAGAGGCCTGGCCAGGGACTGGAGTGGATCGGCG
ACATTAACCCCACAAACGGAGGCACAAACTACAACGAGAAATTCAAGAC
CAAAGCCATCCTGACCGTGGACAGAAGCAGTAGCACAGCCTACATGCAG
GTGAGCAGCCTGACCAGCGAGGACAGCGCCGTGTACTATTGCGCCAGAAG
CGGCGGCTACGACTTCGACTACTGGGGCCAGGGCACCACCCTGACCGTGA
GCAGC
188H6D3-VL
Amino acid sequence (SEQ ID NO: 176):
DVVMTQTPLTLSVTIGQPASISCKSSQSLLDSDGKTYLNWLLQRPGQSPKRLI
YLVSKLDSGVPDRFTGSGSGTDFTLKISRVEAEDLGVYYCWQGTYFPLTFGA
GTKLELK
Nucleic acid sequence (SEQ ID NO: 220):
GACGTGGTGATGACCCAGACCCCCCTGACACTGAGCGTGACCATCGGACA
GCCCGCCAGCATCAGCTGCAAGAGCAGCCAGAGCCTGCTGGACAGCGAC
GGAAAAACATATCTGAATTGGCTGCTGCAGAGGCCTGGCCAGAGCCCCAA
AAGACTGATCTACCTGGTGAGCAAGCTGGACAGCGGGGTGCCCGACAGAT
TCACCGGCAGCGGAAGCGGCACCGACTTCACCCTGAAGATCTCCAGAGTG
GAAGCCGAGGACCTGGGCGTGTACTACTGCTGGCAGGGAACCTACTTCCC
CCTGACCTTTGGAGCCGGCACCAAGCTGGAACTGAAG

CDRs are known to be responsible for antigen binding, however, it has been found that not all of the 6 CDRs are indispensable or unchangeable. In other words, it is possible to replace or change or modify one or more CDRs in anti-CD24 antibody 33E7E12, 71A4F12, 56A7E3, 58F10-1F6, 73H5-1G2, 110D4G4, 81A1F8, 100F2E3, 111F3A2, 81A7A10 107D10D11, 101H9G9A2, 173B1C1, 107D10E11, 109G10A6, 94G12D11, 92F5B8, 185B11E3, 168B11E2 or 188H6D3, yet substantially retain the specific binding affinity to CD24.

In certain embodiments, the anti-CD24 antibodies and the antigen-binding fragments provided herein comprise a heavy chain CDR3 sequence of one of the anti-CD24 antibodies 33E7E12, 71A4F12, 56A7E3, 58F10-1F6, 73H5-1G2, 110D4G4, 81A1F8, 100F2E3, 111F3A2, 81A7A10 107D10D11, 101H9G9A2, 173B1C1, 107D10E11, 109G10A6, 94G12D11, 92F5B8, 185B11E3, 168B11E2 or 188H6D3. In certain embodiments, the anti-CD24 antibodies and the antigen-binding fragments provided herein comprise a heavy chain CDR3 sequence selected from the group consisting of SEQ ID NOs: 1, 3, 5, 7, 9, 11, 14, 16, 17, 18, 19, 21, 23, 25, 27, 28, 30, 32, 34, 36, 38, 40, 42, 44, 46, 48, 97, 104, 105, 106, 113, 114, 115, 126, 127, 128, 136, 137, 150, 151, 152, 160, 161, 162, 168, 169 170, 222, 223 and 224. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the antibodies and/or antigen-binding fragments thereof provided herein comprise suitable framework region (FR) sequences, as long as the antibodies and/or antigen-binding fragments thereof can specifically bind to CD24. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and/or antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623). In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody.

The present disclosure also provides anti-CD24 antibodies and antigen-binding fragments thereof comprising one or more (e.g. 1, 2, 3, 4, 5, or 6) CDR sequences of an humanized anti-CD24 antibody 81A1F8-VH11/VL11, 81A1F8-VH21/VL11, 81A1F8-VH31/VL11, 81A1F8-VH11/VL21, 81A1F8-VH21/VL21, 81A1F8-VH31/VL21, 81A1F8-VH11/VL31, 81A1F8-VH21/VL31, 81A1F8-VH31/VL31, 81A1F8-VH1/VL41, 81A1F8-VH21NL41 or 81A1F8-VH31/VL41; or 101H9G9A2-mVH/mVL-V1, 101H9G9A2-mVH/mVL-V2, 101H9G9A2-mVH/mVL-V3, 101H9G9A2-mVH/mVL-V4, 101H9G9A2-mVH/mVL-V5, 101H9G9A2-hVH1/hVL1, 101H9G9A2-hVH1/hVL2, 101H9G9A2-hVH1/hVL3, 101H9G9A2-hVH1/hVL4, 101H9G9A2-hVH2/hVL1, 101H9G9A2-hVH2/hVL2, 101H9G9A2-hVH2/hVL3, 101H9G9A2-hVH2/hVL4, 101H9G9A2-hVH3/hVL1, 101H9G9A2-hVH3/hVL2, 101H9G9A2-hVH3/hVL3, 101H9G9A2-hVH3/hVL4, 101H9G9A2-hVH4/hVL1, 101H9G9A2-hVH4/hVL2, 101H9G9A2-hVH4/hVL3, 101H9G9A2-hVH4/hVL4, 101H9G9A2-hVH5/hVL1, 101H9G9A2-hVH5/hVL2, 101H9G9A2-hVH5/hVL3 or 101H9G9A2-hVH5/hVL4.

“81A1F8-VH11/VL11” as used herein refers to a humanized antibody based on 81A1F8 mouse/chimeric that comprises a heavy chain variable region of SEQ ID NO: 177, and a light chain variable region of SEQ ID NO: 180.

“81A1F8-VH21/VL11” as used herein refers to a humanized antibody based on 81A1F8 mouse/chimeric that comprises a heavy chain variable region of SEQ ID NO: 178, and a light chain variable region of SEQ ID NO: 180.

“81A1F8-VH31/VL11” as used herein refers to a humanized antibody based on 81A1F8 mouse/chimeric that comprises a heavy chain variable region of SEQ ID NO: 179, and a light chain variable region of SEQ ID NO: 180.

“81A1F8-VH11/VL21” as used herein refers to a humanized antibody based on 81A1F8 mouse/chimeric that comprises a heavy chain variable region of SEQ ID NO: 177, and a light chain variable region of SEQ ID NO: 181.

“81A1F8-VH21/VL21” as used herein refers to a humanized antibody based on 81A1F8 mouse/chimeric that comprises a heavy chain variable region of SEQ ID NO: 178, and a light chain variable region of SEQ ID NO: 181.

“81A1F8-VH31/VL21” as used herein refers to a humanized antibody based on 81A1F8 mouse/chimeric that comprises a heavy chain variable region of SEQ ID NO: 179, and a light chain variable region of SEQ ID NO: 181.

“81A1F8-VH11/VL31” as used herein refers to a humanized antibody based on 81A1F8 mouse/chimeric that comprises a heavy chain variable region of SEQ ID NO: 177, and a light chain variable region of SEQ ID NO: 182.

“81A1F8-VH21/VL31′” as used herein refers to a humanized antibody based on 81A1F8 mouse/chimeric that comprises a heavy chain variable region of SEQ ID NO: 178, and a light chain variable region of SEQ ID NO: 182.

“81 A1F8-VH31/VL31” as used herein refers to a humanized antibody based on 81A1F8 mouse/chimeric that comprises a heavy chain variable region of SEQ ID NO: 179, and a light chain variable region of SEQ ID NO: 182.

“81A1F8-VH11/VL41” as used herein refers to a humanized antibody based on 81A1F8 mouse/chimeric that comprises a heavy chain variable region of SEQ ID NO: 177, and a light chain variable region of SEQ ID NO: 183.

“81A1F8-VH21/VL41” as used herein refers to a humanized antibody based on 81A1F8 mouse/chimeric that comprises a heavy chain variable region of SEQ ID NO: 178, and a light chain variable region of SEQ ID NO: 183.

“81A1F8-VH31/VL41” as used herein refers to a humanized antibody based on 81A1F8 mouse/chimeric that comprises a heavy chain variable region of SEQ ID NO: 179, and a light chain variable region of SEQ ID NO: 183.

“101H9G9A2-mVH/mVL-V1” as used herein refers to a humanized antibody based on 101H9G9A2 mouse/chimeric that comprises a heavy chain variable region of SEQ ID NO: 189, and a light chain variable region of SEQ ID NO: 195.

“101H9G9A2-mVH/mVL-V2” as used herein refers to a humanized antibody based on 101H9G9A2 mouse/chimeric that comprises a heavy chain variable region of SEQ ID NO:189, and a light chain variable region of SEQ ID NO: 196.

“101H9G9A2-mVH/mVL-V3” as used herein refers to a humanized antibody based on 101H9G9A2 mouse/chimeric that comprises a heavy chain variable region of SEQ ID NO: 189, and a light chain variable region of SEQ ID NO: 197.

“101H9G9A2-mVH/mVL-V4” as used herein refers to a humanized antibody based on 101H9G9A2 mouse/chimeric that comprises a heavy chain variable region of SEQ ID NO: 189, and a light chain variable region of SEQ ID NO: 198.

“101H9G9A2-mVH/mVL-V5” as used herein refers to a humanized antibody based on 101H9G9A2 mouse/chimeric that comprises a heavy chain variable region of SEQ ID NO: 189, and a light chain variable region of SEQ ID NO: 199.

“101H9G9A2-hVH1/hVL1” as used herein refers to a humanized antibody based on 101H9G9A2 mouse/chimeric that comprises a heavy chain variable region of SEQ ID NO: 190, and a light chain variable region of SEQ ID NO: 200.

“101H9G9A2-hVH1/hVL2” as used herein refers to a humanized antibody based on 101H9G9A2 mouse/chimeric that comprises a heavy chain variable region of SEQ ID NO: 190, and a light chain variable region of SEQ ID NO: 201.

“101H9G9A2-hVH1/hVL3” as used herein refers to a humanized antibody based on 101H9G9A2 mouse/chimeric that comprises a heavy chain variable region of SEQ ID NO: 190, and a light chain variable region of SEQ ID NO: 202.

“101H9G9A2-hVH1/hVL4” as used herein refers to a humanized antibody based on 101H9G9A2 mouse/chimeric that comprises a heavy chain variable region of SEQ ID NO: 190, and a light chain variable region of SEQ ID NO: 203.

“101H9G9A2-hVH2/hVL1” as used herein refers to a humanized antibody based on 101H9G9A2 mouse/chimeric that comprises a heavy chain variable region of SEQ ID NO: 191, and a light chain variable region of SEQ ID NO: 200.

“101H9G9A2-hVH2/hVL2” as used herein refers to a humanized antibody based on 101H9G9A2 mouse/chimeric that comprises a heavy chain variable region of SEQ ID NO: 191, and a light chain variable region of SEQ ID NO: 201.

“101H9G9A2-hVH2/hVL3” as used herein refers to a humanized antibody based on 101H9G9A2 mouse/chimeric that comprises a heavy chain variable region of SEQ ID NO: 191, and a light chain variable region of SEQ ID NO: 202.

“101H9G9A2-hVH2/hVL4” as used herein refers to a humanized antibody based on 101H9G9A2 mouse/chimeric that comprises a heavy chain variable region of SEQ ID NO: 191, and a light chain variable region of SEQ ID NO: 203.

“101H9G9A2-hVH3/hVL1” as used herein refers to a humanized antibody based on 101H9G9A2 mouse/chimeric that comprises a heavy chain variable region of SEQ ID NO: 192, and a light chain variable region of SEQ ID NO: 200.

“101H9G9A2-hVH3/hVL2” as used herein refers to a humanized antibody based on 101H9G9A2 mouse/chimeric that comprises a heavy chain variable region of SEQ ID NO: 192, and a light chain variable region of SEQ ID NO: 201.

“101H9G9A2-hVH3/hVL3” as used herein refers to a humanized antibody based on 101H9G9A2 mouse/chimeric that comprises a heavy chain variable region of SEQ ID NO: 192, and a light chain variable region of SEQ ID NO: 202.

“101H9G9A2-hVH3/hVL4” as used herein refers to a humanized antibody based on 101H9G9A2 mouse/chimeric that comprises a heavy chain variable region of SEQ ID NO: 192, and a light chain variable region of SEQ ID NO: 203.

“101H9G9A2-hVH4/hVL1” as used herein refers to a humanized antibody based on 101H9G9A2 mouse/chimeric that comprises a heavy chain variable region of SEQ ID NO: 193, and a light chain variable region of SEQ ID NO: 200.

“101H9G9A2-hVH4/hVL2” as used herein refers to a humanized antibody based on 101H9G9A2 mouse/chimeric that comprises a heavy chain variable region of SEQ ID NO: 193, and a light chain variable region of SEQ ID NO: 201.

“101H9G9A2-hVH4/hVL3” as used herein refers to a humanized antibody based on 101H9G9A2 mouse/chimeric that comprises a heavy chain variable region of SEQ ID NO: 193, and a light chain variable region of SEQ ID NO: 202.

“101H9G9A2-hVH4/hVL4” as used herein refers to a humanized antibody based on 101H9G9A2 mouse/chimeric that comprises a heavy chain variable region of SEQ ID NO: 193, and a light chain variable region of SEQ ID NO: 203.

“101H9G9A2-hVH5/hVL1” as used herein refers to a humanized antibody based on 101H9G9A2 mouse/chimeric that comprises a heavy chain variable region of SEQ ID NO: 194, and a light chain variable region of SEQ ID NO: 200.

“101H9G9A2-hVH5/hVL2” as used herein refers to a humanized antibody based on 101H9G9A2 mouse/chimeric that comprises a heavy chain variable region of SEQ ID NO: 194, and a light chain variable region of SEQ ID NO: 201.

“101H9G9A2-hVH5/hVL3” as used herein refers to a humanized antibody based on 101H9G9A2 mouse/chimeric that comprises a heavy chain variable region of SEQ ID NO: 194, and a light chain variable region of SEQ ID NO: 202.

“101H9G9A2-hVH5/hVL4” as used herein refers to a humanized antibody based on 101H9G9A2 mouse/chimeric that comprises a heavy chain variable region of SEQ ID NO: 194, and a light chain variable region of SEQ ID NO: 203.

Table 2 shows the CDR sequences of these 37 humanized anti-CD24 antibodies according to IMGT numbering. The heavy chain and light chain variable region sequences are also provided below.

TABLE 2

| Antibody ID: | | CDR1 | CDR2 | CDR3 |
|---|---|---|---|---|
| 81A1F8-VH11/VL11 | VH | SEQ ID NO: 25 | SEQ ID NO: 30 | SEQ ID NO: 28 |
| 81A1F8-VH21/VL11 | | GYYMN | EINPNTGDTNN | RDYGTSL |
| 81A1F8-VH31/VL11 | | | NQKFKA | |
| 81A1F8-VH11/VL21 | VL | SEQ ID NO: 29 | SEQ ID NO: 22 | SEQ ID NO: 31 |
| 81A1F8-VH21/VL21 | | RASKSINKYLA | SGSTLQS | QQHNEYPIT |
| 81A1F8-VH31/VL21 | | | | |
| 81A1F8-VH11/VL31 | | | | |
| 81A1F8-VH21/VL31 | | | | |
| 81A1F8-VH31/VL31 | | | | |
| 81A1F8-VH11/VL41 | | | | |
| 81A1F8-VH21/VL41 | | | | |
| 81A1F8-VH31/VL41 | | | | |
| 101H9G9A2-mVH/mVL- | VH | SEQ ID NO: 104 | SEQ ID NO: 105 | SEQ ID NO: 106 |
| V1 | | DYTFTSYW | IYPSDSKT | ARRGGYYAYA |
| | | | | MDY |
| 101H9G9A2-mVH/mVL- | VL | SEQ ID NO: 184 | SEQ ID NO: 108 | SEQ ID NO: 37 |
| V1 | | QSLLQSGNQDN | GAR | QNDHSYPYT |
| | | Y | | |
| 101H9G9A2-mVH/mVL- | VH | SEQ ID NO: 104 | SEQ ID NO: 105 | SEQ ID NO: 106 |
| V2 | | DYTFTSYW | IYPSDSKT | ARRGGYYAYA |
| | | | | MDY |
| 101H9G9A2-mVH/mVL- | VL | SEQ ID NO: 185 | SEQ ID NO: 108 | SEQ ID NO: 37 |
| V2 | | QSLLNAGNQD | GAR | QNDHSYPYT |
| | | NY | | |
| 101H9G9A2-mVH/mVL- | VH | SEQ ID NO: 104 | SEQ ID NO: 105 | SEQ ID NO: 106 |
| V3 | | DYTFTSYW | IYPSDSKT | ARRGGYYAYA |
| | | | | MDY |
| | VL | SEQ ID NO: 186 | SEQ ID NO: 108 | SEQ ID NO: 37 |
| | | QSLLSSGNQDN | GAR | QNDHSYPYT |
| | | Y | | |
| 101H9G9A2-m VH/mVL- | VH | SEQ ID NO: 104 | SEQ ID NO: 105 | SEQ ID NO: 106 |
| V4 | | DYTFTSYW | IYPSDSKT | ARRGGYYAYA |
| | | | | MDY |
| 101H9G9A2-m VH/mVL- | VL | SEQ ID NO: 187 | SEQ ID NO: 108 | SEQ ID NO: 37 |
| V4 | | QSLLYSGNQDN | GAR | QNDHSYPYT |
| | | Y | | |
| 101H9G9A2-mVH/mVL- | VH | SEQ ID NO: 104 | SEQ ID NO: 105 | SEQ ID NO: 106 |
| V5 | | DYTFTSYW | IYPSDSKT | ARRGGYYAYA |
| | | | | MDY |
| 101H9G9A2-mVH/mVL- | VL | SEQ ID NO: 188 | SEQ ID NO: 108 | SEQ ID NO: 37 |
| V5 | | QSLLASGNQDN | GAR | QNDHSYPYT |
| | | Y | | |
| 101H9G9A2-hVH1/hVL1 | VH | SEQ ID NO: 104 | SEQ ID NO: 105 | SEQ ID NO: 106 |
| 101H9G9A2-hVH1/hVL2 | | DYTFTSYW | IYPSDSKT | ARRGGYYAYA |
| 101H9G9A2-hVH1/hVL3 | | | | MDY |
| 101H9G9A2-hVH1/hVL4 | VL | SEQ ID NO: 107 | SEQ ID NO: 108 | SEQ ID NO: 37 |
| 101H9G9A2-hVH2/hVL1 | | QSLLNSGNQDN | GAR | QNDHSYPYT |
| 101H9G9A2-hVH2/hVL2 | | Y | | |
| 101H9G9A2-hVH2/hVL3 | | | | |
| 101H9G9A2-hVH2/hVL4 | | | | |
| 101H9G9A2-hVH3/hVL1 | | | | |
| 101H9G9A2-hVH3/hVL2 | | | | |
| 101H9G9A2-hVH3/hVL3 | | | | |
| 101H9G9A2-hVH3/hVL4 | | | | |
| 101H9G9A2-hVH4/hVL1 | | | | |
| 101H9G9A2-hVH4/hVL2 | | | | |
| 101H9G9A2-hVH4/hVL3 | | | | |
| 101H9G9A2-hVH4/hVL4 | | | | |
| 101H9G9A2-hVH5/hVL1 | | | | |
| 101H9G9A2-hVHS/hVL2 | | | | |
| 101H9G9A2-hVHS/hVL3 | | | | |
| 101H9G9A2-hVH5/hVL4 | | | | |

Heavy or light chain variable region sequences of these humanized antibodies are provided below.

81A1F8-VH11/VL11
VH Amino acid sequence (SEQ ID NO: 177):
QVQLVQSGAEVKKPGASVKVSCKASGYLFTGYYMNWVRQAPGQGLEWMG
EINPNTGDTNNNQKFKARVTMTVDRSISTAYMELSRLRSDDTAVYYCVRRD
YGTSLDYWGQGTLVTVSS
VL Amino acid sequence (SEQ ID NO: 180):
DVQITQSPSSLSASVGDRVTITCRASKSINKYLAWYQQKPGKAPKLLIHSGSTL
QSGVPSRFSGSRSGTDFTLTISSLQPEDFATYYCQQHNEYPITFGQGTKLEIK
81A1F8-VH21/VL11
VH Amino acid sequence (SEQ ID NO: 178):
QVQLVQSGAEVKKPGASVKVSCKASGYLFTGYYMNWVRQAPGQGLEWMG
EINPNTGDTNNNQKFKARVTMTVDRSTSTAYMELSSLRSEDTAVYYCVRRD
YGTSLDYWGQGTLVTVSS
VL Amino acid sequence (SEQ ID NO: 180):
DVQITQSPSSLSASVGDRVTITCRASKSINKYLAWYQQKPGKAPKLLIHSGSTL
QSGVPSRFSGSRSGTDFTLTISSLQPEDFATYYCQQHNEYPITFGQGTKLEIK
81A1F8-VH31/VL11
VH Amino acid sequence (SEQ ID NO: 179):
QVQLVQSGAEVKKPGASVKVSCKASGYLFTGYYMNWVRQAPGQRLEWMG
EINPNTGDTNNNQKFKARVTITVDRSASTAYMELSSLRSEDTAVYYCVRRDY
GTSLDYWGQGTLVTVSS
VL Amino acid sequence (SEQ ID NO: 180):
DVQITQSPSSLSASVGDRVTITCRASKSINKYLAWYQQKPGKAPKLLIHSGSTL
QSGVPSRFSGSRSGTDFTLTISSLQPEDFATYYCQQHNEYPITFGQGTKLEIK
81A1F8-VH11/VL21
VH Amino acid sequence (SEQ ID NO: 177):
QVQLVQSGAEVKKPGASVKVSCKASGYLFTGYYMNWVRQAPGQGLEWMG
EINPNTGDTNNNQKFKARVTMTVDRSISTAYMELSRLRSDDTAVYYCVRRD
YGTSLDYWGQGTLVTVSS
VL Amino acid sequence (SEQ ID NO: 181):
DVQITQSPSFLSASVGDRVTITCRASKSINKYLAWYQQKPGKAPKLLIHSGSTL
QSGVPSRFSGSRSGTEFTLTISSLQPEDFATYYCQQHNEYPITFGQGTKLEIK
81A1F8-VH21/VL21
VH Amino acid sequence (SEQ ID NO: 178):
QVQLVQSGAEVKKPGASVKVSCKASGYLFTGYYMNWVRQAPGQGLEWMG
EINPNTGDTNNNQKFKARVTMTVDRSTSTAYMELSSLRSEDTAVYYCVRRD
YGTSLDYWGQGTLVTVSS
VL Amino acid sequence (SEQ ID NO: 181):
DVQITQSPSFLSASVGDRVTITCRASKSINKYLAWYQQKPGKAPKLLIHSGSTL
QSGVPSRFSGSRSGTEFTLTISSLQPEDFATYYCQQHNEYPITFGQGTKLEIK
81A1F8-VH31/VL21
VH Amino acid sequence (SEQ ID NO: 179):
QVQLVQSGAEVKKPGASVKVSCKASGYLFTGYYMNWVRQAPGQRLEWMG
EINPNTGDTNNNQKFKARVTITVDRSASTAYMELSSLRSEDTAVYYCVRRDY
GTSLDYWGQGTLVTVSS
VL Amino acid sequence (SEQ ID NO: 181):
DVQITQSPSFLSASVGDRVTITCRASKSINKYLAWYQQKPGKAPKLLIHSGSTL
QSGVPSRFSGSRSGTEFTLTISSLQPEDFATYYCQQHNEYPITFGQGTKLEIK
81A1F8-VH11/VL31
VH Amino acid sequence (SEQ ID NO: 177):
QVQLVQSGAEVKKPGASVKVSCKASGYLFTGYYMNWVRQAPGQGLEWMG
EINPNTGDTNNNQKFKARVTMTVDRSISTAYMELSRLRSDDTAVYYCVRRD
YGTSLDYWGQGTLVTVSS
VL Amino acid sequence (SEQ ID NO: 182):
DVQITQSPSSLSASVGDRVTITCRASKSINKYLAWYQQKPGKVPKLLIHSGSTL
QSGVPSRFSGSRSGTDFTLTISSLQPEDFATYYCQQHNEYPITFGQGTKLEIK
81A1F8-VH21/VL31
VH Amino acid sequence (SEQ ID NO: 178):
QVQLVQSGAEVKKPGASVKVSCKASGYLFTGYYMNWVRQAPGQGLEWMG
EINPNTGDTNNNQKFKARVTMTVDRSTSTAYMELSSLRSEDTAVYYCVRRD
YGTSLDYWGQGTLVTVSS
VL Amino acid sequence (SEQ ID NO: 182):
DVQITQSPSSLSASVGDRVTITCRASKSINKYLAWYQQKPGKVPKLLIHSGSTL
QSGVPSRFSGSRSGTDFTLTISSLQPEDFATYYCQQHNEYPITFGQGTKLEIK
81A1F8-VH31/VL31
VH Amino acid sequence (SEQ ID NO: 179):
QVQLVQSGAEVKKPGASVKVSCKASGYLFTGYYMNWVRQAPGQRLEWMG
EINPNTGDTNNNQKFKARVTITVDRSASTAYMELSSLRSEDTAVYYCVRRDY
GTSLDYWGQGTLVTVSS
VL Amino acid sequence (SEQ ID NO: 182):
DVQITQSPSSLSASVGDRVTITCRASKSINKYLAWYQQKPGKVPKLLIHSGSTL
QSGVPSRFSGSRSGTDFTLTISSLQPEDFATYYCQQHNEYPITFGQGTKLEIK
81A1F8-VH11/VL41
VH Amino acid sequence (SEQ ID NO: 177):
QVQLVQSGAEVKKPGASVKVSCKASGYLFTGYYMNWVRQAPGQGLEWMG
EINPNTGDTNNNQKFKARVTMTVDRSISTAYMELSRLRSDDTAVYYCVRRD
YGTSLDYWGQGTLVTVSS
VL Amino acid sequence (SEQ ID NO: 183):
DVQITQSPSTLSASVGDRVTITCRASKSINKYLAWYQQKPGKAPKLLIHSGSTL
QSGVPSRFSGSRSGTEFTLTISSLQPDDFATYYCQQHNEYPITFGQGTKLEIK
81A1F8-VH21/VL41
VH Amino acid sequence (SEQ ID NO: 178):
QVQLVQSGAEVKKPGASVKVSCKASGYLFTGYYMNWVRQAPGQGLEWMG
EINPNTGDTNNNQKFKARVTMTVDRSTSTAYMELSSLRSEDTAVYYCVRRD
YGTSLDYWGQGTLVTVSS
VL Amino acid sequence (SEQ ID NO: 183):
DVQITQSPSTLSASVGDRVTITCRASKSINKYLAWYQQKPGKAPKLLIHSGSTL
QSGVPSRFSGSRSGTEFTLTISSLQPDDFATYYCQQHNEYPITFGQGTKLEIK
81A1F8-VH31/VL41
VH Amino acid sequence (SEQ ID NO: 179):
QVQLVQSGAEVKKPGASVKVSCKASGYLFTGYYMNWVRQAPGQRLEWMG
EINPNTGDTNNNQKFKARVTITVDRSASTAYMELSSLRSEDTAVYYCVRRDY
GTSLDYWGQGTLVTVSS
VL Amino acid sequence (SEQ ID NO: 183):
DVQITQSPSTLSASVGDRVTITCRASKSINKYLAWYQQKPGKAPKLLIHSGSTL
QSGVPSRFSGSRSGTEFTLTISSLQPDDFATYYCQQHNEYPITFGQGTKLEIK
101H9G9A2-mVH/mVL-V1
VH Amino acid sequence (SEQ ID NO: 189):
QVQLLQPGAELVRPGSSVKLSCKASDYTFTSYWMDWVKQRPGQGLEWIGNI
YPSDSKTHYNQKFKDKATLTVDKSSSTAYMQLNSLTSEDSAVYYCARRGGY
YAYAMDYWGQGTSVTVSS
VL Amino acid sequence (SEQ ID NO: 195):
DIVMTQSPSSLSVSAGEKVTMRCKSSQSLLQSGNQDNYLAWYQQKPGQPPKL
LLYGARIRESGVPDRFIGSGSGTDFTLTISSVQAEDLAVYYCQNDHSYPYTFG
GGTKVEIK
101H9G9A2-mVH/mVL-V2
VH Amino acid sequence (SEQ ID NO: 189):
QVQLLQPGAELVRPGSSVKLSCKASDYTFTSYWMDWVKQRPGQGLEWIGNI
YPSDSKTHYNQKFKDKATLTVDKSSSTAYMOLNSLTSEDSAVYYCARRGGY
YAYAMDYWGQGTSVTVSS
VL Amino acid sequence (SEQ ID NO: 196):
DIVMTQSPSSLSVSAGEKVTMRCKSSQSLLNAGNQDNYLAWYQQKPGQPPK
LLLYGARIRESGVPDRFIGSGSGTDFTLTISSVQAEDLAVYYCQNDHSYPYTFG
GGTKVEIK
101H9G9A2-mVH/mVL-V3
VH Amino acid sequence (SEQ ID NO: 189):
QVQLLQPGAELVRPGSSVKLSCKASDYTFTSYWMDWVKQRPGQGLEWIGNI
YPSDSKTHYNQKFKDKATLTVDKSSSTAYMQLNSLTSEDSAVYYCARRGGY
YAYAMDYWGQGTSVTVSS
VL Amino acid sequence (SEQ ID NO: 197):
DIVMTQSPSSLSVSAGEKVTMRCKSSQSLLSSGNQDNYLAWYQQKPGQPPKL
LLYGARIRESGVPDRFIGSGSGTDFTLTISSVQAEDLAVYYCQNDHSYPYTFG
GGTKVEIK
101H9G9A2-mVH/mVL-V4
VH Amino acid sequence (SEQ ID NO: 189):
QVQLLQPGAELVRPGSSVKLSCKASDYTFTSYWMDWVKQRPGQGLEWIGNI
YPSDSKTHYNQKFKDKATLTVDKSSSTAYMQLNSLTSEDSAVYYCARRGGY
YAYAMDYWGQGTSVTVSS
VL Amino acid sequence (SEQ ID NO: 198):
DIVMTQSPSSLSVSAGEKVTMRCKSSQSLLYSGNQDNYLAWYQQKPGQPPKL
LLYGARIRESGVPDRFIGSGSGTDFTLTISSVQAEDLAVYYCQNDHSYPYTFG
GGTKVEIK
101H9G9A2-mVH/mVL-V5
VH Amino acid sequence (SEQ ID NO: 189):
QVQLLQPGAELVRPGSSVKLSCKASDYTFTSYWMDWVKQRPGQGLEWIGNI
YPSDSKTHYNQKFKDKATLTVDKSSSTAYMQLNSLTSEDSAVYYCARRGGY
YAYAMDYWGQGTSVTVSS
VL Amino acid sequence (SEQ ID NO: 199):
DIVMTQSPSSLSVSAGEKVTMRCKSSQSLLASGNQDNYLAWYQQKPGQPPKL
LLYGARIRESGVPDRFIGSGSGTDFTLTISSVQAEDLAVYYCQNDHSYPYTFG
GGTKVEIK
101H9G9A2-hVH1/hVL1
VH Amino acid sequence (SEQ ID NO: 190):
QVQLVQSGAEVKKPGASVKVSCKASDYTFTSYWMDWVRQAPGQGLEWMG
NIYPSDSKTHYNQKFKDRVTMTVDKSTSTAYMELSSLRSEDTAVYYCARRG
GYYAYAMDYWGQGTLVTVSS
VL Amino acid sequence (SEQ ID NO: 200):
DIVMTQSPDSLAVSLGERATINCKSSQSLLNSGNQDNYLAWYQQKPGQPPKL
LIYGARIRESGVPDRFSGSGSGTDFTLTISSLQAEDLAVYYCQNDHSYPYTFGG
GTKVEIK
101H9G9A2-hVH1/hVL2
VH Amino acid sequence (SEQ ID NO: 190):
QVQLVQSGAEVKKPGASVKVSCKASDYTFTSYWMDWVRQAPGQGLEWMG
NIYPSDSKTHYNQKFKDRVTMTVDKSTSTAYMELSSLRSEDTAVYYCARRG
GYYAYAMDYWGQGTLVTVSS
VL Amino acid sequence (SEQ ID NO: 201):
DIVMTQSPLSLPVTPGEPASISCKSSQSLLNSGNQDNYLAWYLQKPGQSPQLLI
YGARIRESGVPDRFSGSGSGTDFTLKISRVEAEDLGVYYCQNDHSYPYTFGGG
TKVEIK
101H9G9A2-hVH1/hVL3
VH Amino acid sequence (SEQ ID NO: 190):
QVQLVQSGAEVKKPGASVKVSCKASDYTFTSYWMDWVRQAPGQGLEWMG
NIYPSDSKTHYNQKFKDRVTMTVDKSTSTAYMELSSLRSEDTAVYYCARRG
GYYAYAMDYWGQGTLVTVSS
VL Amino acid sequence (SEQ ID NO: 202):
EIVMTQSPATLSVSPGERATLSCKSSQSLLNSGNQDNYLAWYQQKPGQAPRL
LIYGARIRESGIPARFSGSGSGTEFTLTISSLQSEDLAVYYCQNDHSYPYTFGGG
TKVEIK
101H9G9A2-hVH1/hVL4
VH Amino acid sequence (SEQ ID NO: 190):
QVQLVQSGAEVKKPGASVKVSCKASDYTFTSYWMDWVRQAPGQGLEWMG
NIYPSDSKTHYNQKFKDRVTMTVDKSTSTAYMELSSLRSEDTAVYYCARRG
GYYAYAMDYWGQGTLVTVSS
VL Amino acid sequence (SEQ ID NO: 203):
DIQMTQSPSSLSASVGDRVTITCKSSQSLLNSGNQDNYLAWYQQKPGKAPKL
LIYGARIRESGVPSRFSGSGSGTDFTLTISSLQPEDLATYYCQNDHSYPYTFGG
GTKVEIK
101H9G9A2-hVH2/hVL1
VH Amino acid sequence (SEQ ID NO: 191):
QVQLVQSGAEVKKPGASVKVSCKASDYTFTSYWMDWVRQAPGQRLEWMG
NIYPSDSKTHYNQKFKDRVTITVDKSASTAYMELSSLRSEDTAVYYCARRGG
YYAYAMDYWGQGTLVTVSS
VL Amino acid sequence (SEQ ID NO: 200):
DIVMTQSPDSLAVSLGERATINCKSSQSLLNSGNQDNYLAWYQQKPGQPPKL
LIYGARIRESGVPDRFSGSGSGTDFTLTISSLQAEDLAVYYCQNDHSYPYTFGG
GTKVEIK
101H9G9A2-h VH2/hVL2
VH Amino acid sequence (SEQ ID NO: 191):
QVQLVQSGAEVKKPGASVKVSCKASDYTFTSYWMDWVRQAPGQRLEWMG
NIYPSDSKTHYNQKFKDRVTITVDKSASTAYMELSSLRSEDTAVYYCARRGG
YYAYAMDYWGQGTLVTVSS
VL Amino acid sequence (SEQ ID NO: 201):
DIVMTQSPLSLPVTPGEPASISCKSSQSLLNSGNQDNYLAWYLQKPGQSPQLLI
YGARIRESGVPDRFSGSGSGTDFTLKISRVEAEDLGVYYCQNDHSYPYTFGGG
TKVEIK
101H9G9A2-h VH2/hVL3
VH Amino acid sequence (SEQ ID NO: 191):
QVQLVQSGAEVKKPGASVKVSCKASDYTFTSYWMDWVRQAPGQRLEWMG
NIYPSDSKTHYNQKFKDRVTITVDKSASTAYMELSSLRSEDTAVYYCARRGG
YYAYAMDYWGQGTLVTVSS
VL Amino acid sequence (SEQ ID NO: 202):
EIVMTQSPATLSVSPGERATLSCKSSQSLLNSGNQDNYLAWYQQKPGQAPRL
LIYGARIRESGIPARFSGSGSGTEFTLTISSLQSEDLAVYYCQNDHSYPYTFGGG
TKVEIK
101H9G9A2-hVH2/hVL4
VH Amino acid sequence (SEQ ID NO: 191):
QVQLVQSGAEVKKPGASVKVSCKASDYTFTSYWMDWVRQAPGQRLEWMG
NIYPSDSKTHYNQKFKDRVTITVDKSASTAYMELSSLRSEDTAVYYCARRGG
YYAYAMDYWGQGTLVTVSS
VL Amino acid sequence (SEQ ID NO: 203):
DIQMTQSPSSLSASVGDRVTITCKSSQSLLNSGNQDNYLAWYQQKPGKAPKL
LIYGARIRESGVPSRFSGSGSGTDFTLTISSLQPEDLATYYCQNDHSYPYTFGG
GTKVEIK
101H9G9A2-hVH3/hVL1
VH Amino acid sequence (SEQ ID NO: 192):
QVQLVQSGAEVKKPGASVKVSCKASDYTFTSYWMDWVRQAPGQGLEWMG
NIYPSDSKTHYNQKFKDRVTMTVDKSISTAYMELSRLRSDDTAVYYCARRGG
YYAYAMDYWGQGTLVTVSS
VL Amino acid sequence (SEQ ID NO: 200):
DIVMTQSPDSLAVSLGERATINCKSSQSLLNSGNQDNYLAWYQQKPGQPPKL
LIYGARIRESGVPDRFSGSGSGTDFTLTISSLQAEDLAVYYCQNDHSYPYTFGG
GTKVEIK
101H9G9A2-hVH3/hVL2
VH Amino acid sequencE (SEQ ID NO: 192):
QVQLVQSGAEVKKPGASVKVSCKASDYTFTSYWMDWVRQAPGQGLEWMG
NIYPSDSKTHYNQKFKDRVTMTVDKSISTAYMELSRLRSDDTAVYYCARRGG
YYAYAMDYWGQGTLVTVSS
VL Amino acid sequence (SEQ ID NO: 201):
DIVMTQSPLSLPVTPGEPASISCKSSQSLLNSGNQDNYLAWYLQKPGQSPQLLI
YGARIRESGVPDRFSGSGSGTDFTLKISRVEAEDLGVYYCQNDHSYPYTFGGG
TKVEIK
101H9G9A2-h VH3/hVL3
VH Amino acid sequence (SEQ ID NO: 192):
QVQLVQSGAEVKKPGASVKVSCKASDYTFTSYWMDWVRQAPGQGLEWMG
NIYPSDSKTHYNQKFKDRVTMTVDKSISTAYMELSRLRSDDTAVYYCARRGG
YYAYAMDYWGQGTLVTVSS
VL Amino acid sequence (SEQ ID NO: 202):
EIVMTQSPATLSVSPGERATLSCKSSQSLLNSGNQDNYLAWYQQKPGQAPRL
LIYGARIRESGIPARFSGSGSGTEFTLTISSLQSEDLAVYYCQNDHSYPYTFGGG
TKVEIK
101H9G9A2-hVH3/hVL4
VH Amino acid sequence (SEQ ID NO: 192):
QVQLVQSGAEVKKPGASVKVSCKASDYTFTSYWMDWVRQAPGQGLEWMG
NIYPSDSKTHYNQKFKDRVTMTVDKSISTAYMELSRLRSDDTAVYYCARRGG
YYAYAMDYWGQGTLVTVSS
VL Amino acid sequence (SEQ ID NO: 203):
DIQMTQSPSSLSASVGDRVTITCKSSQSLLNSGNQDNYLAWYQQKPGKAPKL
LIYGARIRESGVPSRFSGSGSGTDFTLTISSLQPEDLATYYCQNDHSYPYTFGG
GTKVEIK
101H9G9A2-hVH4/hVL1
VH Amino acid sequence (SEQ ID NO: 193):
QVQLVQSGAEVKKPGSSVKVSCKASDYTFTSYWMDWVRQAPGQGLEWMG
NIYPSDSKTHYNQKFKDRVTITVDKSTSTAYMELSSLRSEDTAVYYCARRGG
YYAYAMDYWGQGTLVTVSS
VL Amino acid sequence (SEQ ID NO: 200):
DIVMTQSPDSLAVSLGERATINCKSSQSLLNSGNQDNYLAWYQQKPGQPPKL
LIYGARIRESGVPDRFSGSGSGTDFTLTISSLQAEDLAVYYCQNDHSYPYTFGG
GTKVEIK
101H9G9A2-hVH4/hVL2
VH Amino acid sequence (SEQ ID NO: 193):
QVQLVQSGAEVKKPGSSVKVSCKASDYTFTSYWMDWVRQAPGQGLEWMG
NIYPSDSKTHYNQKFKDRVTITVDKSTSTAYMELSSLRSEDTAVYYCARRGG
YYAYAMDYWGQGTLVTVSS
VL Amino acid sequence (SEQ ID NO: 201):
DIVMTQSPLSLPVTPGEPASISCKSSQSLLNSGNQDNYLAWYLQKPGQSPQLLI
YGARIRESGVPDRFSGSGSGTDFTLKISRVEAEDLGVYYCQNDHSYPYTFGGG
TKVEIK
101H9G9A2-h VH4/hVL3
VH Amino acid sequence (SEQ ID NO: 193):
QVQLVQSGAEVKKPGSSVKVSCKASDYTFTSYWMDWVRQAPGQGLEWMG
NIYPSDSKTHYNQKFKDRVTITVDKSTSTAYMELSSLRSEDTAVYYCARRGG
YYAYAMDYWGQGTLVTVSS
VL Amino acid sequence (SEQ ID NO: 202):
EIVMTQSPATLSVSPGERATLSCKSSQSLLNSGNQDNYLAWYQQKPGQAPRL
LIYGARIRESGIPARFSGSGSGTEFTLTISSLQSEDLAVYYCQNDHSYPYTFGGG
TKVEIK
101H9G9A2-hVH4/hVL4
VH Amino acid sequence (SEQ ID NO: 193):
QVQLVQSGAEVKKPGSSVKVSCKASDYTFTSYWMDWVRQAPGQGLEWMG
NIYPSDSKTHYNQKFKDRVTITVDKSTSTAYMELSSLRSEDTAVYYCARRGG
YYAYAMDYWGQGTLVTVSS
VL Amino acid sequence (SEQ ID NO: 203):
DIQMTQSPSSLSASVGDRVTITCKSSQSLLNSGNQDNYLAWYQQKPGKAPKL
LIYGARIRESGVPSRFSGSGSGTDFTLTISSLQPEDLATYYCQNDHSYPYTFGG
GTKVEIK
101H9G9A2-hVH5/hVL1
VH Amino acid sequence (SEQ ID NO: 194):
QVQLVQSGAEVKKPGASVKVSCKASDYTFTSYWMDWVRQAPGQGLEWMG
NIYPSDSKTHYNQKFKDRVTMTVDKSTSTAYMELRSLRSDDTAVYYCARRG
GYYAYAMDYWGQGTLVTVSS
VL Amino acid sequence (SEQ ID NO: 200):
DIVMTQSPDSLAVSLGERATINCKSSQSLLNSGNQDNYLAWYQQKPGQPPKL
LIYGARIRESGVPDRFSGSGSGTDFTLTISSLQAEDLAVYYCQNDHSYPYTFGG
GTKVEIK
101H9G9A2-hVH5/hVL2
VH Amino acid sequence (SEQ ID NO: 194):
QVQLVQSGAEVKKPGASVKVSCKASDYTFTSYWMDWVRQAPGQGLEWMG
NIYPSDSKTHYNQKFKDRVTMTVDKSTSTAYMELRSLRSDDTAVYYCARRG
GYYAYAMDYWGQGTLVTVSS
VL Amino acid sequence (SEQ ID NO: 201):
DIVMTQSPLSLPVTPGEPASISCKSSQSLLNSGNQDNYLAWYLQKPGQSPQLLI
YGARIRESGVPDRFSGSGSGTDFTLKISRVEAEDLGVYYCQNDHSYPYTFGGG
TKVEIK
101H9G9A2-hVH5/hVL3
VH Amino acid sequence (SEQ ID NO: 194):
QVQLVQSGAEVKKPGASVKVSCKASDYTFTSYWMDWVRQAPGQGLEWMG
NIYPSDSKTHYNQKFKDRVTMTVDKSTSTAYMELRSLRSDDTAVYYCARRG
GYYAYAMDYWGQGTLVTVSS
VL Amino acid sequence (SEQ ID NO: 202):
EIVMTQSPATLSVSPGERATLSCKSSQSLLNSGNQDNYLAWYQQKPGQAPRL
LIYGARIRESGIPARFSGSGSGTEFTLTISSLQSEDLAVYYCQNDHSYPYTFGGG
TKVEIK
101H9G9A2-hVH5/hVL4
VH Amino acid sequence (SEQ ID NO: 194):
QVQLVQSGAEVKKPGASVKVSCKASDYTFTSYWMDWVRQAPGQGLEWMG
NIYPSDSKTHYNQKFKDRVTMTVDKSTSTAYMELRSLRSDDTAVYYCARRG
GYYAYAMDYWGQGTLVTVSS
VL Amino acid sequence (SEQ ID NO: 203):
DIQMTQSPSSLSASVGDRVTITCKSSQSLLNSGNQDNYLAWYQQKPGKAPKL
LIYGARIRESGVPSRFSGSGSGTDFTLTISSLQPEDLATYYCQNDHSYPYTFGG
GTKVEIK

In certain embodiments, the anti-CD24 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In some embodiments, the anti-CD24 antibodies and antigen-binding fragments thereof provided herein have a constant region of an immunoglobulin (Ig), optionally a human Ig, optionally a human IgG. In some embodiments, the anti-CD24 antibodies and antigen-binding fragments thereof provided herein comprises a constant region of human IgG1, IgG2, IgG3, or IgG4.

Human IgG isotypes (the subclasses of mature gamma globulin class G antibodies; IgG1, IgG2, IgG3 and IgG4) exhibit differential capacity to recruit effector functions. For example, ADCC is promoted by IgG1 and IgG3, ADCP is promoted by IgG1, IgG2, IgG3 and IgG4, and CDC is promoted by IgG1 and IgG3. Isotype-specific engagement of such effector functions is based on selectivity for Fc receptors on distinct immune cells and the ability to bind C1q thereby activating the assembly of a membrane attack complex (MAC). Among the various isotypes, relative affinity for Fcγ receptors, which include FcγRI, FcγRIIa/b/c, and FcγRIIIa/b is high for IgG1 and IgG3. However, Fcγ affinity for IgG2 is considerably lower with the exception of FcγRIIa H131 polymorphism and IgG4 only has measurable affinity for FcγRI.

In certain embodiments, the anti-CD24 antibodies and antigen-binding fragments thereof provided herein comprises a constant region of IgG1 isotype, which could induce ADCC, CDC or ADCP, or a constant region of IgG4 or IgG2 isotype, which has reduced or depleted effector function. Effector functions such as ADCC and CDC can lead to cytotoxicity to cells expressing CD24. Effector functions can be evaluated using various assays such as Fc receptor binding assay, C1q binding assay, and cell lysis assay.

Antibody Variants

The present disclosure also encompasses various variants of the antibodies and/or antigen-binding fragments thereof provided herein. In certain embodiments, the present disclosure encompasses various types of variants of an exemplary antibody provided herein, i.e., 33E7E12, 71A4F12, 56A7E3, 58F10-1F6, 73H5-1G2, 110D4G4, 81 A1F8, 100F2E3, 111F3A2, 81A7A10 or 107D10D11.

In certain embodiments, the antibody variants comprise one or more modifications or substitutions in one or more CDR sequences as provided in Table 1, one or more FR sequences, the heavy or light chain variable region sequences provided herein, and/or the constant region (e.g. Fc region). Such variants retain specific binding affinity to CD24 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

Affinity Variant

Affinity variant may contain modifications or substitutions in one or more CDR sequences as provided in Table 1, one or more FR sequences, or the heavy or light chain variable region sequences provided herein. The affinity variants retain specific binding affinity to CD24 of the parent antibody, or even have improved CD24 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences provided in Table 1 and FR sequences, one or more amino acid residues may be substituted yet the resulting antibody or antigen-binding fragment still retain the binding affinity to CD24, or even have an improved binding affinity. Various methods known in the art can be used to achieve this purpose. For example, a library of antibody variants (such as Fab or scFv variants) can be generated and expressed with phage display technology, and then screened for the binding affinity to human CD24. For another example, computer software can be used to virtually simulate the binding of the antibodies to human CD24, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-CD24 antibodies and antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CD24 at a level similar to or even higher than its parent antibody.

In certain embodiments, the anti-CD24 antibodies and antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CD24 at a level similar to or even higher than its parent antibody. In some embodiments, a total of 1 to 10 amino acids have been substituted, inserted, or deleted in a sequence selected from that (or those) listed in Table 1. In some embodiments, the substitutions, insertions, or deletions occur in regions outside the CDRs (i.e., in the FRs).

Glycosylation Variant

The anti-CD24 antibodies and antigen-binding fragments provided herein also encompass a glycosylation variant, which can be obtained to either increase or decrease the extent of glycosylation of the antibody or antigen binding fragment.

The antibody or antigen binding fragment thereof may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

Cysteine-Engineered Variant

The anti-CD24 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisotope among others, at the site of the engineered cysteine, through for example a maleimide or haloacetyl. Methods for engineering antibodies or antigen-binding fragments to introduce free cysteine residues are known in the art, see, for example, WO2006/034488.

Fc Variant

The anti-CD24 antibodies and antigen-binding fragments provided herein also encompass an Fc variant, which comprises one or more amino acid residue modifications or substitutions at its Fc region and/or hinge region.

In certain embodiments, the anti-CD24 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the anti-CD24 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276(9): 6591-604; Idusogie E E. et al., J Immunol. 2000. 164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the anti-CD24 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants.

In certain embodiments, the anti-CD24 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

Antigen-Binding Fragments

Provided herein are also anti-CD24 antigen-binding fragments. Various types of antigen-binding fragments are known in the art and can be developed based on the anti-CD24 antibodies provided herein, including for example, the exemplary antibodies whose CDR and FR sequences are shown in Table 1, and their different variants (such as affinity variants, glycosylation variants, Fc variants, cysteine-engineered variants and so on).

In certain embodiments, an anti-CD24 antigen-binding fragment provided herein is a camelized single domain antibody, a diabody, a single chain Fv fragment (scFv), an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, a single domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxy terminus to provide for a fusion protein (see, for example, Antibody Engineering, ed. Borrebaeck).

Bispecific Antibodies, Multivalent Antibodies

In certain embodiments, the antibodies or antigen-binding fragments thereof provided herein are bivalent, tetravalent, hexavalent, or multivalent. In certain embodiments, the antibodies and/or antigen-binding fragments thereof provided herein are monospecific, or bispecific.

The term “valent” as used herein refers to the presence of a specified number of antigen binding sites in a given molecule. As such, the terms “bivalent”, “tetravalent”, and “hexavalent” denote the presence of two binding sites, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope).

In certain embodiments, the antibodies or antigen-binding fragments thereof provided herein can be monospecific but bivalent, trivalent, or tetravalent, with at least two binding sites specific for the same antigen or epitope. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences), or structurally different (i.e. having different sequences albeit with the same specificity).

In certain embodiments, the antibodies or antigen-binding fragments thereof provided herein are bispecific. In some embodiments, the bispecific antibodies and antigen-binding fragments thereof provided herein binds to a first and a second epitope of CD24, wherein the first and the second epitope of CD24 are different.

In some embodiments, the bispecific antibodies or antigen-binding fragments thereof provided herein binds to a first epitope of CD24, and a second epitope of an antigen other than CD24.

The bispecific antibody molecules provided herein can be in any suitable bispecific format known in the art. Examples of reference bispecific antibody formats known in the art include, without limitation, (i) a bispecific antibody with symmetric Fc, (ii) a bispecific antibody with asymmetric Fc, (iii) a regular antibody appended with an additional antigen-binding moiety, (iv) a bispecific antibody fragment, (v) a regular antibody fragment appended with an additional antigen-binding moiety, (vi) a bispecific antibody appended with human albumin or human albumin-binding peptide.

Bispecific IgG-like antibodies (BsIgG) is monovalent for each antigen and can be produced by co-expression of the two light and two heavy chains in a single host cell. An appending IgG is engineered to form bispecific IgG by appending either the amino or carboxyl termini of either light or heavy chains with additional antigen-binding units. The additional antigen-binding units can be single domain antibodies (unpaired VL or VH), such as DVD-Ig, paired antibody variable domains (e.g. Fv or scFv) or engineered protein scaffolds.

Bispecific antibody fragments are antigen-binding fragments that are derived from an antibody but lack some or all of the antibody constant domains. Examples of such a bispecific antibody fragment include, for example, such as single domain antibody, Fv, Fab and diabody etc.

In certain embodiments, the bispecific antibody molecules as provided herein are based on the format of a “whole” antibody, such as whole IgG or IgG-like molecules, and small recombinant formats, such as tandem single chain variable fragment molecules (taFvs), diabodies (Dbs), single chain diabodies (scDbs) and various other derivatives of these (cf. bispecific antibody formats as described by Byrne H. et al. (2013) Trends Biotech, 31 (11): 621-632. Examples of bispecific antibody is based on a format which include, but is not limited to, quadroma, chemically coupled Fab (fragment antigen binding), and BiTE (bispecific T cell engager).

In certain embodiments, the bispecific antibody molecules as provided herein are in a bispecific format selected from Triomabs; hybrid hybridoma (quadroma); Multispecific anticalin platform (Pieris); Diabodies; Single chain diabodies; Tandem single chain Fv fragments; TandAbs, Trispecific Abs (Affimed); Darts (dual affinity retargeting; Macrogenics); Bispecific Xmabs (Xencor); Bispecific T cell engagers (Bites; Amgen; 55 kDa); Triplebodies; Tribody (Fab-scFv) Fusion Protein (CreativeBiolabs) multifunctional recombinant antibody derivates; Duobody platform (Genmab); Dock and lock platform; Knob into hole (KIH) platform; Humanized bispecific IgG antibody (REGN1979) (Regeneron); Mab2 bispecific antibodies (F-Star); DVD-Ig (dual variable domain immunoglobulin) (Abbvie); kappa-lambda bodies; TBTI (tetravalent bispecific tandem Ig); and CrossMab.

In certain embodiments, the bispecific antibody molecules as provided herein are in a bispecific format selected from BsIgG comprising CrossMab; DAF (two-in-one); DAF (four-in-one); DutaMab; DT-IgG; Knobs-in-holes common LC; Knobs-in-holes assembly; Charge pair; Fab-arm exchange; SEEDbody; Triomab; LUZ-Y; Fcab; kappa-lamda-body; and Orthogonal Fab. For detailed description of the bispecific antibody formats please see Spiess C., Zhai Q. and Carter P. J. (2015) Molecular Immunology 67: 95-106, which is incorporated herein by reference to its entirety.

In certain embodiments, the bispecific antibody molecules as provided herein are in a bispecific format selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)—IgG; IgG(L)-V; V(L)-IgG; KIH IgG-scFab; 2scFv-IgG; IgG-2scFv; scFv4-Ig; scFv4-Ig; Zybody; and DVI-IgG (four-in-one) (see Id.).

In certain embodiments, the bispecific antibody molecules as provided herein are in a format selected from bispecific antibody fragments comprising Nanobody; Nanobody-HAS; BiTE; Diabody; DART; TandAb; scDiabody; sc-Diabody-CH3; Diabody-CH3; Triple Body; Miniantibody; Minibody; TriBi minibody; scFv-CH3 KIH; Fab-scFv; scFv-CH-CL-scFv; F(ab′)2; F(ab′)2-scFv2; scFv-KIH; Fab-scFv-Fc; Tetravalent HCAb; scDiabody-Fc; Diabody-Fc; Tandem scFv-Fc; and Intrabody (see Id.).

In certain embodiments, the bispecific antibody molecules as provided herein are in a bispecific format such as Dock and Lock; ImmTAC; HSAbody; scDiabody-HAS; and Tandem scFv-Toxin (see Id.).

In certain embodiments, the bispecific antibody molecules as provided herein are based on a format selected from bispecific antibody conjugates comprising IgG-IgG; Cov-X-Body; and scFv1-PEG-scFv2 (see Id.).

In certain embodiments, the second antigen-binding domain is operably linked to the N terminus or the C terminus of the CD24-binding domain. In certain embodiments, the CD24-binding domain is operably linked to the N terminus or the C terminus of the second antigen-binding domain.

The operable linkage can be a direct chemical bond linkage or an indirect linkage via a spacer or via an intervening sequence. The term “spacer” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more binding domains, such as a scFv and a Fab or IgG. The spacer can be identical to or different from the peptide linker in the scFv. In certain embodiment, the spacer comprises 1, 2, 3, 4 or more sequential or tandem repeats of SEQ ID NOs: 90, 91, 92 and 93. In certain embodiments, the spacer comprises GGGGS (SEQ ID NO: 90). In certain embodiments, the spacer comprises GGGGSGGGGS (SEQ ID NO: 91), GGGGSGGGGSGGGGS (SEQ ID NO: 92), GGGGSGGGGSGGGGSGGGGS (SEQ ID NO: 93). The intervening sequence as used herein can be any amino acid sequence located between the CD24-binding domain and the second antigen-binding domain, as long as both the CD24-binding domain and the second antigen-binding domain are capable of binding to its respective antigen. In an illustrative example, the intervening sequence can comprise a heavy chain constant region, or a light chain constant region.

In certain embodiments, the second antigen-binding domain comprises a scFv and the CD24-binding domain comprises a Fab or IgG. In certain embodiments, the second antigen-binding scFv can be operably linked to the N terminus or the C-terminus of the heavy chain of the CD24-binding Fab or IgG (e.g. the C-terminus of the heavy chain constant region following the CD24-binding Fab), or to the N terminus or the C-terminus of the light chain of the CD24-binding Fab or IgG, or any combination thereof, and vice versa.

In an illustrative example, the bispecific antibody molecule can comprise a heavy chain in the format of: VH(anti-CD24)-CH1-Hinge-CH2-CH3-spacer-second antigen-binding scFv or second antigen-binding scFv-spacer-VH(anti-CD24)-CH1-Hinge-CH2-CH3, and a light chain in the format of VL(anti-CD24)-CL. As used herein, VH(anti-CD24) and VL(anti-CD24) refer respectively to the heavy and light chain variable domain of the CD24 binding domain; second antigen-binding scFv refers to scFv of the second antigen-binding domain, CL refers to the light chain constant region; and CH1-Hinge-CH2-CH3 are collectively heavy chain constant region.

In another illustrative example, the bispecific antibody molecule can comprise a light chain in the format of: second antigen-binding scFv-spacer-VL(anti-CD24)-CL or VL(anti-CD24)-CL-spacer-second antigen-binding scFv, and a heavy chain in the format of VH(anti-CD24)-CH1-Hinge-CH2-CH3, by the same token.

In certain embodiments, the CD24-binding domain comprises scFv and the second antigen-binding domain comprises Fab or IgG. In certain embodiments, the CD24-binding domain scFv can be operably linked to the N terminus or the C-terminus of the heavy chain of the second antigen-binding Fab or IgG (e.g. the C-terminus of the heavy chain constant region following the CD24-binding Fab), or to the N terminus or the C-terminus of the light chain of the second antigen-binding Fab or IgG, or any combination thereof, and vice versa.

In an illustrative example, the bispecific antibody molecule can comprise a heavy chain in the format of: second antigen-binding VH-CH1-Hinge-CH2-CH3-spacer-scFv(anti-CD24) or scFv(anti-CD24)-spacer-second antigen-binding VH-CH1-Hinge-CH2-CH3, and a light chain in the format of second antigen-binding VL-CL, by the same token. In another illustrative example, the bispecific antibody molecule can comprise a light chain in the format of: scFv (anti-CD24)-spacer-second antigen-binding VL-CL or second antigen-binding VL-CL-spacer-scFv (anti-CD24), and a heavy chain second antigen-binding VH-CH1-Hinge-CH2-CH3, by the same token.

In the bispecific antibody molecule provided herein, the second antigen-binding domain may be monovalent (i.e. one scFv or Fab) or multivalent (e.g. more than one scFv or Fab), and/or the CD24-binding domain may be monovalent or multivalent.

In certain embodiments, the second antigen is an immune related target, optionally selected from the group consisting of: PD-L1, PD-L2, PD-1, CLTA-4, TIM-3, LAG3, CD160, 2B4, TGF R, VISTA, BTLA, TIGIT, LAIR1, OX40, CD2, CD27, ICAM-1, NKG2C, SLAMF7, NKp80, CD160, B7-H3, LFA-1, ICOS, 4-1BB, GITR, CD30, CD40, BAFFR, HVEM, CD7, LIGHT, IL-2, IL-15, CD3, CD16, SIRPα, Siglec 10, LILRB2, Clever-1, Macro, LILRB4, Siglec15, CSF1R, PSGL-1, VSIG-4, B2M and CD83.

In certain embodiments, the second antigen is a tumor associated antigen or an epitope thereof. The term “tumor associated antigen” used interchangeably with the term “tumor antigen”, refers to an antigen that is or can be presented on a tumor cell surface and that is located on or within tumor cells. In some embodiments, the tumor associated antigens can be presented only by tumor cells and not by normal, i.e. non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but is overexpressed on tumor cells when compared to non-tumor cells or are accessible for antibody binding in tumor cells due to the less compact structure of the tumor tissue compared to non-tumor tissue.

In some embodiments, the tumor antigen is selected from the group consisting of CA-125, gangliosides G(D2), G(M2) and G(D3), CD19, CD20, CD33, CD47, CD52, Ep-CAM, CEA, CLDN18.2, bombesin-like peptides, PSA, HER2/neu, epidermal growth factor receptor (EGFR), erbB2, erbB3/HER3, erbB4, CD44v6, CD44v9, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R, CDH6, CEA, FOLR1, TROP2, PTK7, SLITRK6, CD142, NECTIN-4, ROR1, ROR2, CD142, CD123, CD22, CD79b, DLL3, SLC family or CO17-1A.

In certain embodiments, the second antigen is an immunoinhibitory molecule selected from the group consisting of PD-L1, SIRPα, CD47 and B2M.

In certain embodiments, the second antigen is CD47.

In some embodiments, the CD47-binding scFv is operably linked to the C terminus of the heavy chain constant region following the CD24-binding Fab.

In certain embodiments, the bispecific antibody molecule comprise a heavy chain in the format of: VH(anti-CD24)-CH1-Hinge-CH2-CH3-spacer-scFv(anti-CD47), associated with a light chain in the format of VL(anti-CD24)-CL.

In certain embodiments, the CD47-binding scFv is 5F9 derived scFv and comprises an amino acid sequence of SEQ ID NO: 94 (QVQLVQSGAEVKKPGASVKVSCKASGYTFTNYNMHWVRQAPGQRLEWMG TIYPGNDDTSYNQKFKDRVTITADTSASTAYMELSSLRSEDTAVYYCARGGY RAMDYWGQGTLVTVSSGGGGSGGGGSGGGGSDIVMTQSPLSLPVTPGEPASI SCRSSQSIVYSNGNTYLGWYLQKPGQSPQLLIYKVSNRFSGVPDRFSGSGSGT DFTLKISRVEAEDVGVYYCFQGSHVPYTFGQGTKLEIK).

Other illustrative examples of a tumor associated antigen are CD10, CD21, CD22, CD25, CD30, CD34, CD37, CD38, CD123, GPRC5D, CD44v6, CD45, CD133, Fms-like tyrosine kinase 3 (FLT-3, CD135), chondroitin sulfate proteoglycan 4 (CSPG4, melanoma-associated chondroitin sulfate proteoglycan), Her2, Her3, IGFR, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-alpha (CD140a), PDGFR-beta (CD140b), Endoglin, CLEC14, CLEC12a, Tem1-8, and Tie2. Further examples may include A33, CAMPATH-1 (CDw52), Carcinoembryonic antigen (CEA), Carboanhydrase IX (MN/CA IX), de2-7, EGFRvIII, EpCAM, Folate-binding protein, G250, c-Kit (CD117), CSF1R (CD115), HLA-DR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72.

The bispecific antibodies and antigen-binding fragments provided herein can be made with any suitable methods known in the art. In a conventional approach, two immunoglobulin heavy chain-light chain pairs having different antigenic specificities can be co-expressed in a host cell to produce bispecific antibodies in a recombinant way (see, for example, Milstein and Cuello, Nature, 305: 537 (1983)), followed by purification by affinity chromatography.

Recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g. Gruber et al., J. Immunol., 152:5368 (1994).)

In another method, leucine zipper peptides from the Fos and Jun proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980; WO 91/00360, WO 92/00373, and EP 03089). For another example, the two antibodies or antigen-binding fragments can be cross-linked by conventional methods known in the art, for example, as disclosed in U.S. Pat. No. 4,676,980.

Bispecific antigen-binding fragments may be generated from a bispecific antibody, for example, by proteolytic cleavage, or by chemical linking. For example, an antigen-binding fragment (e.g. Fab′) of an antibody may be prepared and converted to Fab′-thiol derivative and then mixed and reacted with another converted Fab′ derivative having a different antigenic specificity to form a bispecific antigen-binding fragment (see, for example, Brennan et al., Science, 229: 81 (1985)).

In certain embodiments, the bispecific antibody or antigen-binding fragments may be engineered at the interface so that a knob-into-hole association can be formed to promote heterodimerization of the two different antigen-binding sites. “Knob-into-hole” as used herein, refers to an interaction between two polypeptides (such as CH3 domain), where one polypeptide has a protuberance (i.e. “knob”) due to presence of an amino acid residue having a bulky side chain (e.g. tyrosine or tryptophan), and the other polypeptide has a cavity (i.e. “hole”) where a small side chain amino acid residue resides (e.g. alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

Conjugates

In some embodiments, the anti-CD24 antibodies and antigen-binding fragments thereof is linked to one or more conjugates, optionally, wherein the conjugate is covalently attached either directly or via a linker. A conjugate is a non-proteinaceous moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In some embodiments, the conjugate comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In certain embodiments, the antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the antibodies may be linked to a conjugate indirectly, or through another conjugate. For example, the antibody or antigen-binding fragments may be conjugated to biotin, then indirectly conjugated to a second conjugate that is conjugated to avidin. The conjugate can be a toxin (e.g., a chemotherapeutic agent), a detectable label (e.g., a radioactive isotope, a lanthanide, a luminescent label, a fluorescent label, or an enzyme-substrate label).

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luciferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵Sm, ²¹²Bi, and ³²P, other lanthanides, luminescent labels), chromophoric moiety, digoxigenin, biotin/avidin, a DNA molecule or gold for detection.

In certain embodiments, the conjugate can be a pharmacokinetic modifying moiety which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate can be a purification moiety such as a magnetic bead.

In certain embodiments, the antibodies and/or antigen-binding fragments thereof provided herein is used for a base for a conjugate.

Polynucleotides and Recombinant Methods

The present disclosure provides isolated polynucleotides that encode the anti-CD24 antibodies and antigen-binding fragments thereof. In certain embodiments, the isolated polynucleotides comprise one or more nucleotide sequences as shown in SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 102, SEQ ID NO: 110, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 228, SEQ ID NO: 229 and/or SEQ ID NO: 220, which encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CD24 antibodies and antigen-binding fragments thereof (e.g. including the sequences in as shown in SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 102, SEQ ID NO: 110, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 228, SEQ ID NO: 229 and/or SEQ ID NO: 220) can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

In some embodiments, the vector system includes mammalian, bacterial, yeast systems, etc, and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMD18-T, pMONO, pSELECT, pUNO, pDUO, Psg5L, pBABE, pWPXL, pBI, p15TV-L, pPro18, pTD, pRS420, pLexA, pACT2.2 etc, and other laboratorial and commercially available vectors. Suitable vectors may include, plasmid, or viral vectors (e.g., replication defective retroviruses, adenoviruses and adeno-associated viruses).

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CD24 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N Y. Acad. Sci. 383:44-68 (1982)); MRC 5 cells; FS4 cells; and a human hepatoma line (Hep G2).

Host cells are transformed with the above-described expression or cloning vectors for anti-CD24 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD24 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX.™. resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

CAR

In another aspect, the present disclosure also provides chimeric antigen receptor (CAR) comprising an antigen binding domain, a transmembrane domain, a costimulatory signaling region, and a TCR signaling domain, wherein the antigen binding domain specifically binds to CD24 and comprises an antigen binding fragment provided herein. In certain embodiments, the antigen binding fragment is a Fab or a scFv. In certain embodiments, the CAR provided herein is bispecific. The CAR is capable of further specifically binding to a second antigen other than CD24, or a second epitope on CD24. In certain embodiments, the second antigen is a tumor antigen, tumor associated antigen, or an immune related target as mentioned above. The TCR signaling domain can be selected from the group consisting of: an intracellular signal regions sequence of CD3ζ, FccRIγ, CD27, CD28, CD137, CD134, MyD88, CD40, CD278, TLRs, or a combination thereof. The transmembrane region can comprise a transmembrane region of CD3, CD4, CD8 or CD28. In certain embodiments, the second antigen is an immunoinhibitory molecule, such as PD-L1, SIRPα, CD47 or B2M.

In another aspect, the present disclosure also provides a nucleic acid sequence encoding the CAR as mentioned above as well as a cell or a vector comprising such nucleic acid sequence or a cell genetically modified to express the CAR provided herein. The cell can be an immune cell, optionally, wherein the immune cell is T lymphocyte, NK cell, monocyte, macrophage or NKT lymphocyte.

Pharmaceutical Composition

The present disclosure further provides pharmaceutical compositions comprising the anti-CD24 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcellulose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CD24 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Methods of Use

The present disclosure also provides therapeutic methods comprising: administering a therapeutically effective amount of the antibody or antigen-binding fragment as provided herein to a subject in need thereof, thereby treating or preventing a CD24-related disease or condition. In some embodiment, the CD24-related disease or condition is cancer, autoimmune disease, inflammatory disease, adaptive immune disease or infectious disease.

Examples of cancer include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In certain embodiments, the cancer is lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, adenocarcinoma, leukemia, myeloma and lymphoma. In certain embodiments, the cancer is chemoresistant.

In certain embodiments, the disease or condition is hematological cancer chosen from B-cell lymphomas. Examples of B-cell lymphomas includes but not limited to, Hodgkin's lymphoma, non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), diffuse large B cell lymphoma (DLBCL), Marginal zone B-cell lymphoma (MZL), Mantle cell lymphoma (MCL), Richter's syndrome, Burkitt's lymphoma or follicular lymphoma.

Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis herpetiformis; chronic fatigue immune dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury. In some embodiments, the CD24 associated conditions are inflammatory diseases such as systemic lupus erythematosus (SLE), intestinal mucosal inflammation, wasting disease associated with colitis, multiple sclerosis, viral infections, rheumatoid arthritis, osteoarthritis, Cohn's disease, and inflammatory bowel disease, psoriasis, systemic scleroderma, autoimmune diabetes and the like.

Infectious disease include, but are not limited to, fungus infection, parasite/protozoan infection or chronic viral infection, for example, malaria, coccidioidomycosis immitis, histoplasmosis, onychomycosis, aspergillosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fascioloidiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic viruse I, human T lymphotrophic viruse II, varicella zoster, JC virus or BK virus.

In certain embodiments, the subject is human.

In another aspect, methods are provided to treat a disease or condition in a subject that would benefit from modulation of CD24 activity, comprising administering a therapeutically effective amount of the antibody or antigen-binding fragment as provided herein to a subject in need thereof. The term “disease or condition” as used herein can be used interchangeably with the term “CD24-related disease or condition”.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, an antibody or antigen-binding fragment as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. In some embodiments, the antibodies or antigen-binding fragments disclosed herein may be administered alone or in combination with a second therapeutic agent. For example, the antibodies or antigen-binding fragments disclosed herein may be administered in combination with a second therapeutic agent, for example, a chemotherapeutic agent or an anti-cancer drug. In certain embodiments, the antibodies or antigen-binding fragments disclosed herein may be administered in combination with an antagonist of one or more immunoinhibitory molecule, e.g., CD47, SIRPα, PD-L1, or the beta-2 microglobulin subunit of the major histocompatibility class I complex (B2M). The term “antagonist” as used herein comprises can refer to any small molecule, small or micro RNAs, or antibodies or antigen-binding fragments thereof that blocks or inhibits binding of CD47, SIRPα, PD-L1 or B2M to their respective binding partners so as to prevent elicit of immunoinhibitory signals. In certain embodiments, the antibodies or antigen-binding fragments disclosed herein is administered in combination with a CD47 antagonist, such as a SIRPα-Fc fusion protein or variant thereof, an anti-CD47 antibody or antigen-binding fragment thereof. In certain embodiments, the antibodies or antigen-binding fragments disclosed herein is administered in combination with a SIRPα antagonist, such as a soluble CD47 or variant thereof, an anti-SIRPα antibody or an antigen-binding fragment thereof.

The term “anti-CD47 antibody” can refer to any known anti-CD47 antibodies or antigen-binding fragment thereof, including without limitation, Hu5F9 as disclosed in U.S. Pat. No. 9,382,320B2. The Hu5F9-G4 is also used, which is an anti-human CD47 antibody and was humanized by grafting its complementarity determining regions (CDRs) onto a human IgG4 format and results in the humanized 5F9 antibody (Hu5F9-G4) (Liu J. et al., PLoS One, 2015, 10: e0137345).

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In some embodiments, the present disclosure provides methods of detecting presence or amount of CD24 in a sample, comprising contacting the sample with the antibody or antigen-binding fragment thereof, and determining the presence or the amount of CD24 in the sample.

In some embodiments, the present disclosure provides methods of diagnosing a CD24 related disease or condition in a subject, comprising: a) obtaining a sample from the subject; b) contacting the sample obtained from the subject with the antibody or antigen-binding fragment thereof provided herein; c) determining presence or amount of CD24 in the sample; and d) correlating the existence of the CD24 to the CD24 related disease or condition in the subject.

In some embodiments, the present disclosure also provides use of the antibody or antigen-binding fragment thereof provided herein in the manufacture of a medicament for treating a CD24 related disease or condition in a subject, in the manufacture of a diagnostic reagent for diagnosing a CD24 related disease or condition.

In another aspect, the present disclosure also provides a method of modulating CD24 activity in a CD24-expressing cell, comprising exposing the CD24-expressing cell to the antibody or antigen-binding fragment thereof provided herein.

In another aspect, the present disclosure also provides a method of detecting presence or amount of CD24 in a sample, comprising contacting the sample with the antibody or antigen-binding fragment thereof provided herein, and determining the presence or the amount of CD24 in the sample.

In another aspect, the present disclosure also provides a method of diagnosing a CD24 related disease or condition in a subject, comprising: a) obtaining a sample from the subject; b) contacting the sample obtained from the subject with the antibody or antigen-binding fragment thereof provided herein; c) determining presence or amount of CD24 in the sample; and d) correlating the presence or the amount of CD24 to existence or status of the CD24 related disease or condition in the subject.

In another aspect, the present disclosure also provides use of the antibody or antigen-binding fragment thereof provided herein in the manufacture of a medicament for treating a CD24 related disease or condition in a subject. The medicament can further comprise a second therapeutic agent, e.g., a CD47 antagonist, optionally, wherein the CD47 antagonist is a SIRPα-Fc fusion protein or variant thereof, or an anti-CD47 antibody or antigen-binding fragment thereof.

In another aspect, the present disclosure also provides use of the antibody or antigen-binding fragment thereof provided herein in the manufacture of a diagnostic reagent for diagnosing a CD24 related disease or condition.

In another aspect, the present disclosure also provides a kit comprising the antibody or antigen-binding fragment thereof provided herein, useful in detecting CD24, optionally recombinant CD24, CD24 expressed on cell surface, or CD24-expressing cells. The term “recombinant” as used herein refers to artificial manipulation of one or more biological molecules such as polynucleotide or polypeptide molecules using one or more molecular biology techniques to make such biological molecule(s) into something other than its natural state.

In another aspect, the present disclosure also provides a method for stimulating a T cell-mediated immune response to a CD24-expressing cell or tissue in a mammal, the method comprising administering to the mammal an effective amount of a cell genetically modified to express the CAR comprising an antigen binding domain, a transmembrane domain, a costimulatory signaling region, and a TCR signaling domain, wherein the antigen binding domain specifically binds to CD24 and comprises an antigen binding fragment provided herein. In certain embodiments, the antigen binding fragment is a Fab or a scFv. In certain embodiments, the CAR provided herein is bispecific. The CAR is capable of further specifically binding to a second antigen other than CD24 or a second epitope on CD24. In certain embodiments, the second antigen is a tumor antigen as mentioned above. The TCR signaling domain can be selected from the group consisting of: an intracellular signal region sequence of CD3ζ, FccRIγ, CD27, CD28, CD137, CD134, MyD88, CD40, CD278, TLRs, or a combination thereof. The transmembrane region can comprise a transmembrane region of CD3, CD4, CD8 or CD28. In certain embodiments, the second antigen is an immunoinhibitory molecule, e.g., PD-L1, SIRPα, CD47 or B2M.

In another aspect, the present disclosure also provides method for treating a mammal having a CD24 related disease or condition, comprising administering to the mammal an effective amount of a cell that is genetically modified to express the CAR provided herein (e.g., an autologous T cell), thereby treating the mammal. In certain embodiments, the CD24 related disease or condition is cancer. In certain embodiments, the mammal is a human subject.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Hybridoma Development

1. Methods

1.1 Immunization and Serum Titer Determination

1.1.1 Immunogen and Immunization Strategies

Cell immunization: Human CD24 overexpressing cells (HEK293F-hCD24)

Genetic immunization: Full length human CD24 expression construct

Protein immunization: Recombinant human CD24 protein

Balb/c and SJL mice were immunized as shown in Table 2. The primary immunization were followed by several boosts until animals developed satisfactory antiserum titers suitable for hybridoma development. All the immunization strategies were carried out in parallel in order to compare the performance and immune response in serum level.

TABLE 2

| | | | Animal/ | Group | |
|---|---|---|---|---|---|
| Group | Immunogen | Route | Strain | size | Dosage |
| 1 | hCD24 cells | I.P. | SJL | 5 | 0.5-1 × 10⁷ |
| | | | | | cells |
| 2 | hCD24 cells | I.P. | Balb/c | 5 | 0.5-1 × 10⁷ |
| | | | | | cells |
| 3 | hCD24 expression | Gene | SJL | 5 | 4 μg |
| | construct | gun | | | |
| 4 | hCD24 expression | Gene | Balb/c | 5 | 4 μg |
| | construct | gun | | | |
| 5 | Recombinant hCD24 | I.P | SJL | 5 | 25-50 μg |
| | protein | | | | |

1.1.2 Immunization Schedules

TABLE 3
Immunization schedule (Cell immunization)

| Day 0 | Pre-bleed (15-30 μL serum/mouse) |
|---|---|
| | Primary: 0.5-1 × 10⁶/mouse IP |
| 14 | Boost 1: 0.5-1 × 10⁷ cell per mouse IP |
| 21 | Test Bleed (15-30 μL serum/mouse) (TB1) |
| 22 | Test bleed FACS |
| 35 | Boost 2: IP, 0.5-1 × 10⁷ cell per mouse IP |
| 42 | Test Bleed (15-30 AμL serum/mouse) (TB2) |
| 43 | Test bleed FACS |
| 44 | Data analysis and phase conclusion |
| 56 | Pre-fusion (final) Boost, IP, 0.5-1 × 10⁷ cell per mouse |

Animals not selected for cell fusion will be maintained in cage and may be given additional boost immunizations.

TABLE 4
Immunization schedule (Genetic immunization)

| Day 0 | Pre-bleed 15-30 μL serum/mouse) |
|---|---|
| | Primary immunization, 1 μg/shot, 4 shots/animal |
| 14 | Boost 1: 1 μg/shot, 4 shots/animal |
| 21 | Test Bleed (15-30 μL serum/mouse) |
| 22 | Test bleed FACS |
| 28 | Boost 2: 1 μg/shot, 4 shots/animal |
| 35 | Test Bleed (15-30 μL serum/mouse) |
| 36 | Test bleed FACS |
| 42 | Boost 3: 1 μg/shot, 4 shots/animal |
| 49 | Test Bleed (15-30 μL serum/mouse) |
| 50 | Test bleed FACS |
| 51 | Data analysis and phase conclusion |
| 63 | Pre-fusion (final) Boost: 0.5-1 × 10⁷ per mouse |
| 67 | Fusion |

Animals not selected for cell fusion will be maintained in cage and may be given additional boost immunizations.

TABLE 5
Immunization schedule (Protein immunization)

| Day 0 | Pre-bleed (15-30 μL serum/mouse) |
|---|---|
| | Primary: 50 μg/mouse IP, CFA |
| 14 | Boost 1: 25 μg/mouse IP, IFA |
| 21 | Test Bleed (15-30 μL serum/mouse) (TB1) |
| 22 | Test bleed FACS |
| 35 | Boost 2: IP, 25 μg/mouse IP, IFA |
| 42 | Test Bleed (15-30 μL serum/mouse) (TB2) |
| 43 | Test bleed FACS |
| 44 | Data analysis and phase conclusion |
| 56 | Pre-fusion (final) Boost, IP, 25 μg/mouse |

Animals not selected for cell fusion will be maintained in cage andmay be given additional boost immunizations.

1.1.3 Test Bleed Antiserum Analysis

Screening: Test bleeds were performed and evaluated by testing using FACS on 293F cell line stably over-expressing human CD24 (293F-hCD24) and MCF7 cells.

1.2 Hybridoma Generation and Screening

1.2.1 Cell Fusion and Screening

Fusion: Splenocyte fusions were performed on the mice which responded the best to the immunizations as determined by test bleed FACS. The lymphocytes from spleens and lymph nodes were fused to a Sp2/0-Ag14 cell line using an optimized electrofusion protocol. Multiple fusions were performed to ensure success of the project.

Screening and expansion: The fusion was plated (10⁴ to 10⁵ per well) into a stack of 96-well plates. Plates were monitored for growth and fed weekly. Wells with cell growth were screened by primary screening assays in 10-14 days with Acumen and/or other feasible assays. Multiple fusions for each targeting antigen were performed and be screened by Acumen. The positive parental clones which showed positive binding with 293F-hCD24 from primary screening were expanded into 24-well plates for secondary screening.

Additional antibody screening: Following primary screening, positive parental clones expanded into 24-well plates were screened again by the assay described in the hybridoma screening funnel below.

Hybridomas of interest were chosen to proceed to subcloning.

1.2.2 Hybridoma Subcloning, Screening and Cryopreservation

Subcloning: The parental hybridomas with desired reactivity and isotypes from the screening funnel above were then subcloned by multiple rounds of limiting dilution or single cell sorting until mono-clones are obtained.

Screening & Expansion: The subcloning plates were screened by Acumen assay and the subclones with good binding ability were expanded to 24-wells for confirmation tests. The specificity and cross-reactivity of these subclones were confirmed with FACs analysis. Briefly, parental 293F cells, 293F-hCD24, breast cancer cells (MCF7, MDA-MB-468), bladder cancer cells (5637), chronic myelocytic leukemia cells (Nalm1), CKOK1-hCD24 cells, colorectal cancer cells (HT29), ovarian cancer cells (SKOV3), and Lung carcinoma cells (H1975 and A549) were incubated with antibodies produced by each subclone respectively. Fluorescent dye-conjugated secondary antibody was used to detect the binding of the primary antibody with the cells. Median fluorescence intensity (MFI) was measured by FACs analysis.

Cryopreservation—The desired sub-clonal cell lines were sequenced and further expanded into culture flasks for cryopreservation. 4-6 vials per cell line at 0.5-1.0×10⁷ cells/vial were initially cryopreserved. Master cell bank and working cell bank could be established for the selected most valuable cell lines if desired.

2. Results

We discovered a number of antibodies with unique sequences showing positive binding with 293F cell stably over-expressing human CD24 protein (293F-hCD24). The FACS MFI of the antibodies staining 293F cells, 293F-hCD24, breast cancer cells (MCF7, MDA-MB-468), bladder cancer cells (5637), chronic myelocytic leukemia cells (Nalm1), CKOK1-hCD24 cells, colorectal cancer cells (HT29), ovarian cancer cells (SKOV3), and Lung carcinoma cells (H1975 and A549) were summarized in the Table 6 below. Two clones 110D4G4 and 81A1F8 were also applied to stain DLBCL cells OCI-LY1, OCI-LY7 and SU-DHL-10 cells too (Table 7).

TABLE 6
MFI of antibodies binding with different cell lines

| | | | | FACS | | |
|---|---|---|---|---|---|---|
| | | | | MFI | | |
| | FACS | FACS | FACS | MDA- | FACS | FACS |
| | MFI | MFI | MFI | MIB- | MFI | MFI |
| | 293F | 293F- | MCF7 | 468 | Nalm1 | 5637 |
| Clone | ctrl | hCD24 | cells | cells | cells | cells |
| mIgG ctrl | 176 | 185 | 187 | 190 | 175 | 185 |
| SN3 | 187 | 6560 | 1077 | 6692 | 11830 | 9396 |
| 33E7E12 | 182 | 24235 | 5412 | 10907 | 45524 | 14097 |
| 71A4F12 | 499 | 7351 | 2425 | 32523 | 45015 | 18649 |
| 56A7E3 | 833 | 26425 | 9191 | 15265 | 74602 | 18461 |
| 58F10-1F6 | 774 | 7421 | 17352 | 49793 | 20923 | 34233 |
| 73H5-1G2 | 466 | 20125 | 373 | 6404 | 58514 | 6358 |
| 110D4G4 | 1225 | 56479 | 21225 | 52775 | 76230 | 47100 |
| 81A7A10 | 529 | 54424 | 3838 | 45877 | 74041 | 23184 |
| 111F3A2 | 382 | 58833 | 2251 | 35842 | 73783 | 18221 |
| 100F2E3 | 249 | 44060 | 7162 | 43976 | 58827 | 22138 |
| 81A1F8 | 698 | 10301 | 11477 | 24869 | 24797 | 22198 |

TABLE 7
MFI of antibodies binding with different cell lines

| | FACS | | | | FACS | FACS | FACS |
|---|---|---|---|---|---|---|---|
| | MFI | FACS | FACS | FACS | MFI | MFI | MFI |
| | CHOK1- | MFI | MFI | MFI | OCI- | OCI- | SU- |
| Clone | hCD24 | HT-29 | A549 | H1975 | LY1 | LY7 | DHL6 |
| mIgG ctrl | 177 | 194 | 167 | 203 | 197 | 186 | 188 |
| SN3 | 29701 | 5897 | 8820 | 5206 | | | |
| 33E7E12 | 24117 | 2453 | | 6113 | | | |
| 71A4F12 | 28808 | 4780 | 4409 | 7195 | | | |
| 56A7E3 | 36721 | 1461 | 3319 | 2189 | | | |
| 58F10-1F6 | 28095 | 4902 | 12604 | 17462 | | | |
| 73H5-1G2 | 19885 | 4817 | 5696 | 2198 | | | |
| 110D4G4 | 28908 | 5610 | 18352 | 24926 | 4353 | 2930 | 3286 |
| 81A7A10 | 27774 | 5890 | 12218 | 10855 | | | |
| 111F3A2 | 33600 | 4332 | 16253 | 10533 | | | |
| 100F2E3 | 22261 | 2419 | 8667 | 6898 | | | |
| 81A1F8 | 45923 | 3053 | 8659 | 10644 | 5339 | 3855 | 3824 |

Compared with SN3, several of our antibodies showed much higher MFI on the cells tested. Most of our antibodies showed stronger signals on CD24 low-expression MCF7 cells.

### Example 2: Antibody Characterization: Affinity

1. Methods

1.1 Cell Based Binding Affinity on Human CD24

Sequences of some of antibodies from Table 1 were selected to generate and produce human IgG4 chimeric antibodies. The binding affinity of these antibodies and bench mark antibody, SN3 (see. e.g., T Fukukawa et al., Exp Hematol. 1986 October; 14(9):850-5) with 293F-hCD24 cells and human breast cancer cells (MCF7, MDA-MB-468), bladder cancer cells(5637), chronic myelocytic leukemia cells (Nalm1), CKOK1-hCD24 cells, colorectal cancer cells(HT29), ovarian cancer cells(SKOV3), and Lung carcinoma cells (H1975 and A549), was determined by FACs analysis. The protocol for FACs analysis is described as follows:
a. Digested cells using TrypLE™ Express Enzyme (IX); Centrifuged the harvested cells at 400 g for 5 min and discarding the supernatant.
b. Washed the cells 2 times with cold FACS buffer by centrifuging at 400 g for 5 min and discarding the supernatant.
c. Resuspended the cells, and 2*10⁵ cell/well was seeded into the assay plate in 50 ul FACS buffer, then added 50 μl primary antibody (primary antibody final concentration: 50.00, 16.67, 5.56, 1.85, 0.62, 0.21, 0.07, 0.02, 0.01, 0.00 μg/ml). Incubate at 40° C. for 1 h.
d. Washed the cells twice by using the condition in step b. Resuspended the cells with 100 μl/well diluted 2nd antibody, incubated at 4 degree for 1 h in the dark.
e. Washed the cells twice by using the condition in step b. Resuspended the cells with 100 μl/well cold FACS buffer. Kept the cells in dark for FACS analysis.

2. Result

The binding affinity of the antibodies provided herein on 293F-hCD24 cells and human breast cancer cell line, breast cancer cells (MCF7 and MDA-MB-468), bladder cancer cells (5637), chronic myelocytic leukemia cells (Nalm1), acute lymphoblastic leukemia cells (Nalm6), CKOK1-hCD24 cells, colorectal cancer cells (HT29), ovarian cancer cells (SKOV3), lung carcinoma cells (NCI-H1975 and A549), B cell lymphoma cells (SU-DHL6 and DoHH2), human B cells and hepatocellular carcinoma cells (Huh7) were tested. Some were comparable with bench mark antibody SN3. However, our mAb-110D4G4 and mAb-81A7F8 behaved much better than that of SN3 (Table 8A-8B).

Compared with SN3, the max MFI (primary antibody concentration at 50 ug/ml) of some antibodies (110D4G4, 81A1F8) on CD24-low expressing cell MCF7 and A549 was higher than that of SN3. (Table 8 and Table 9).

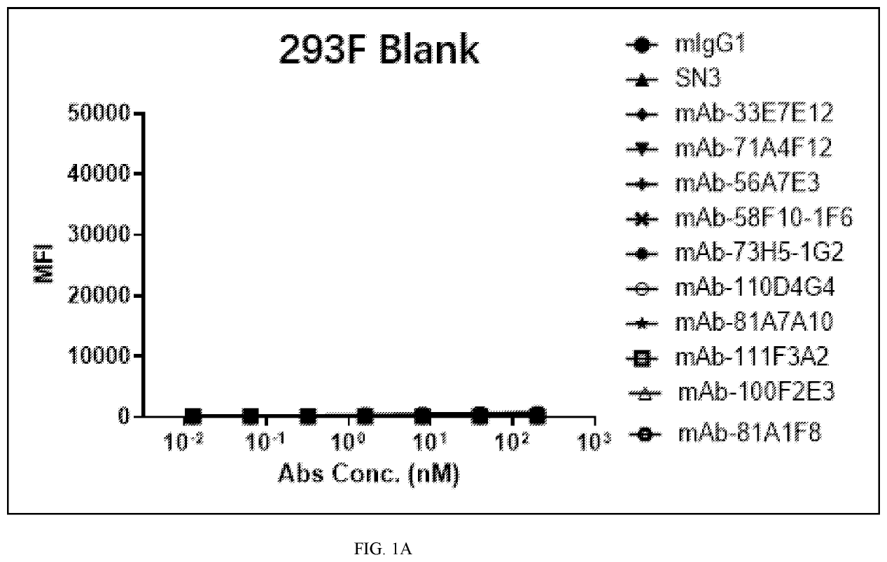

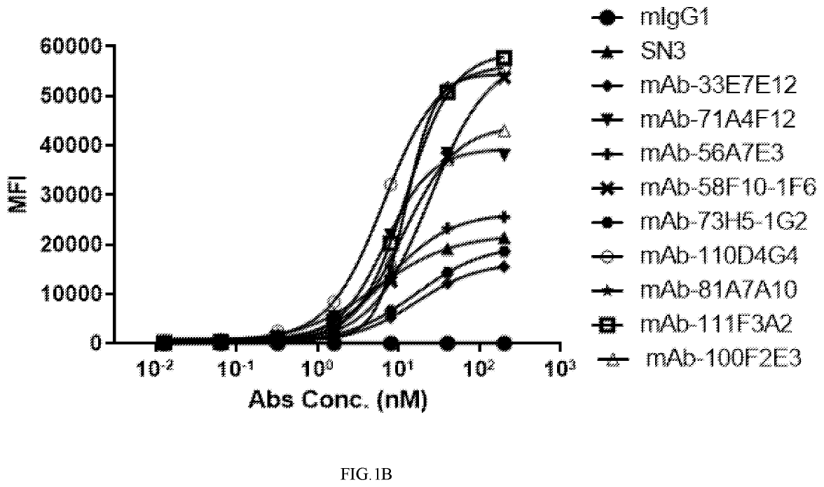

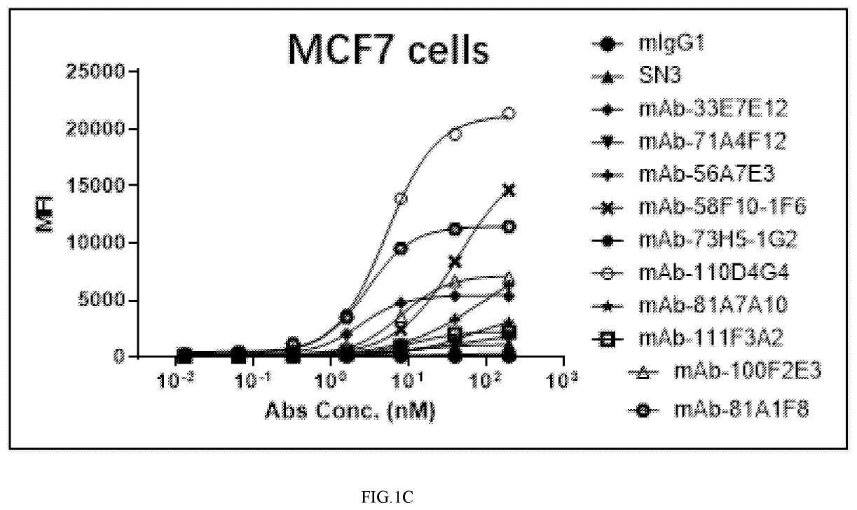

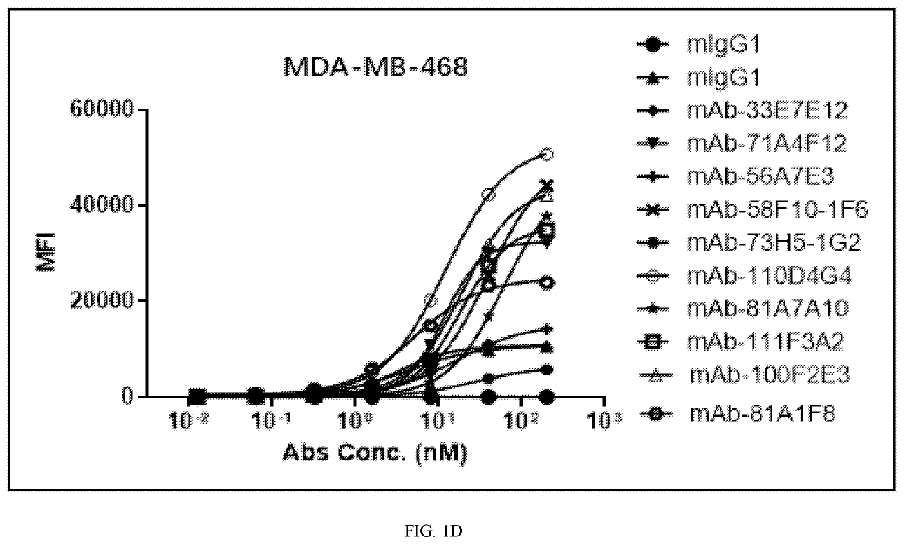

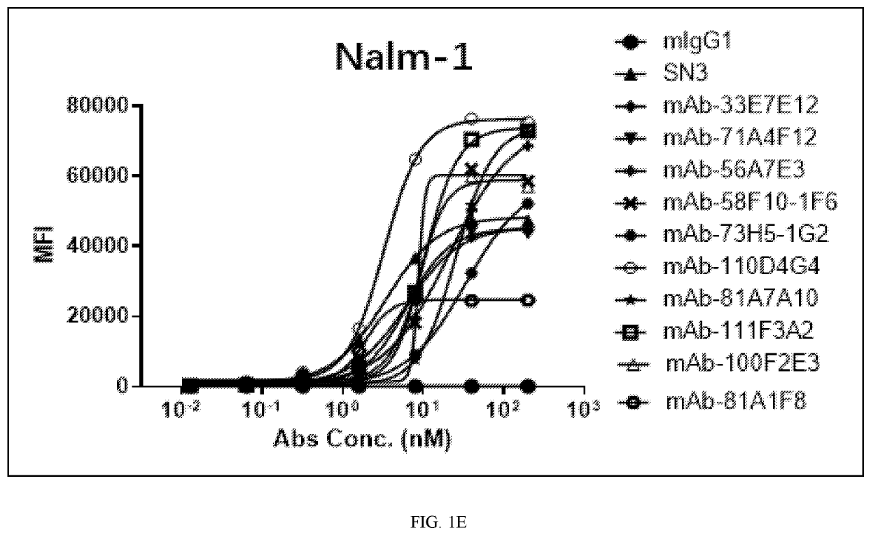

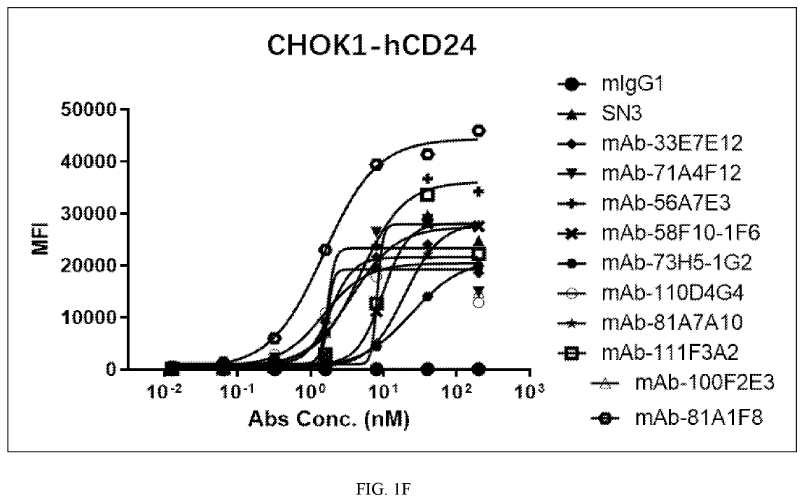

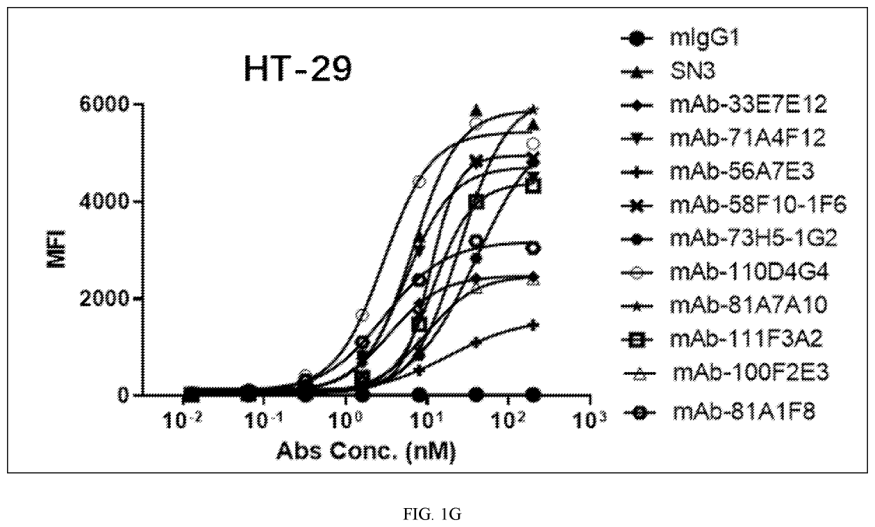

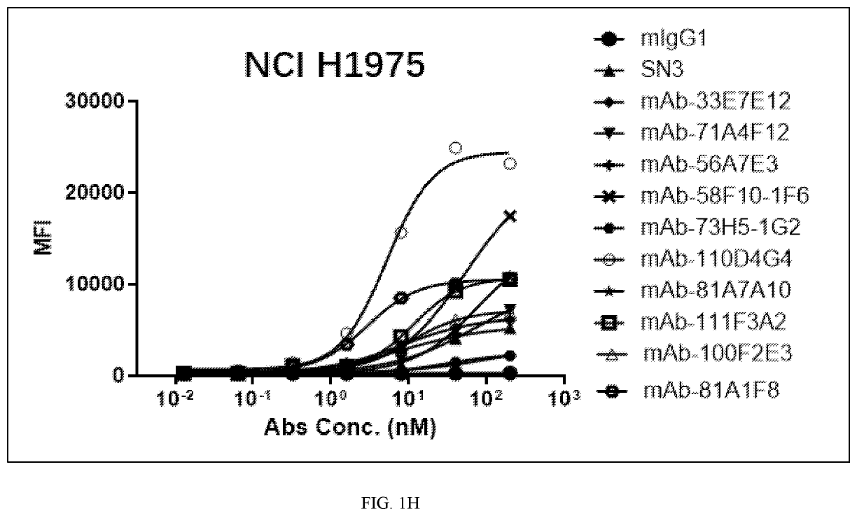

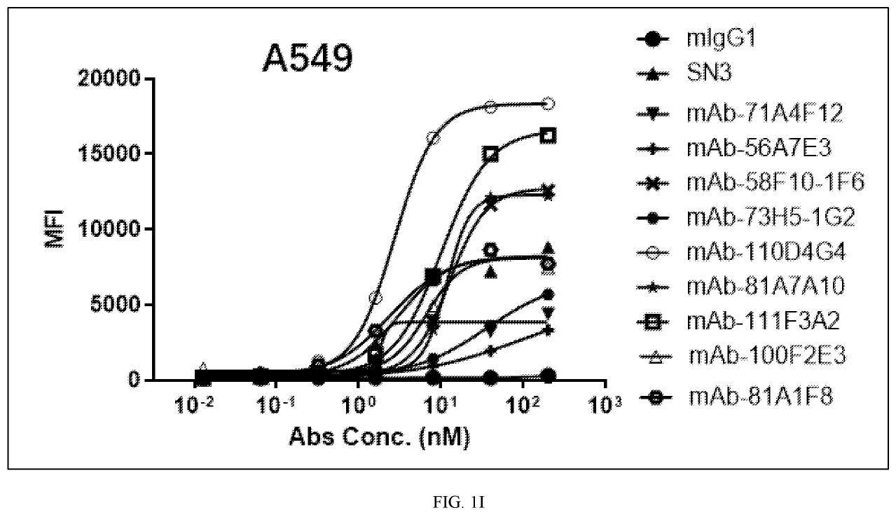

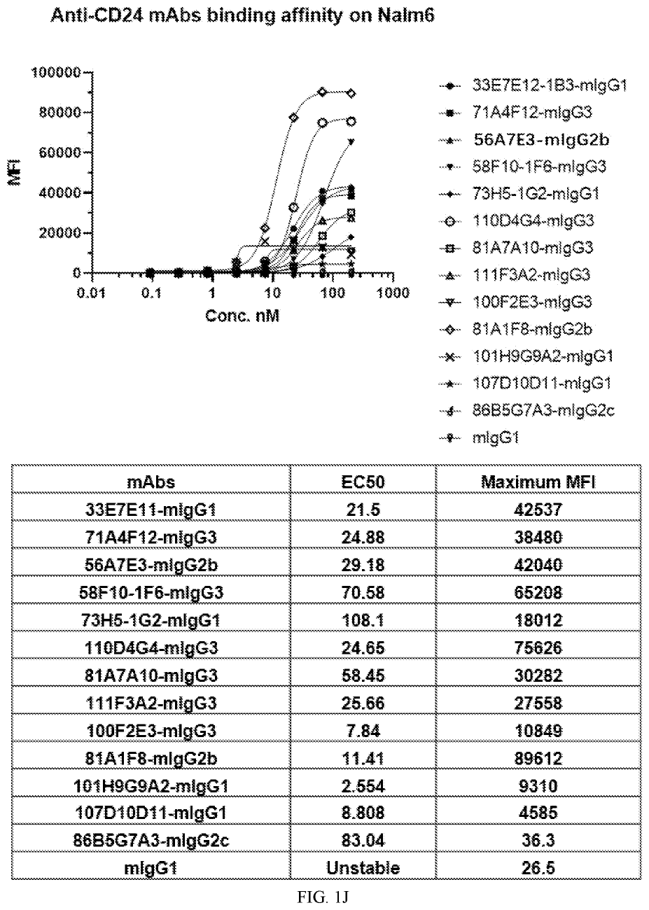

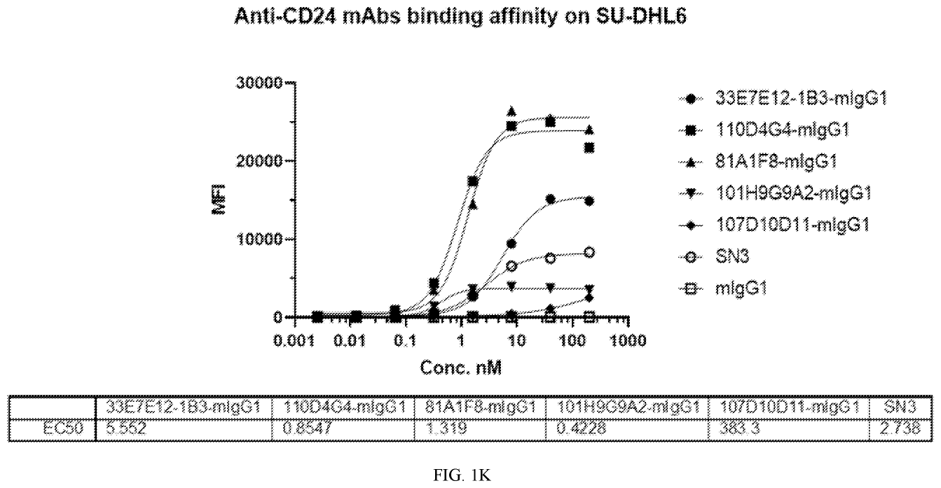

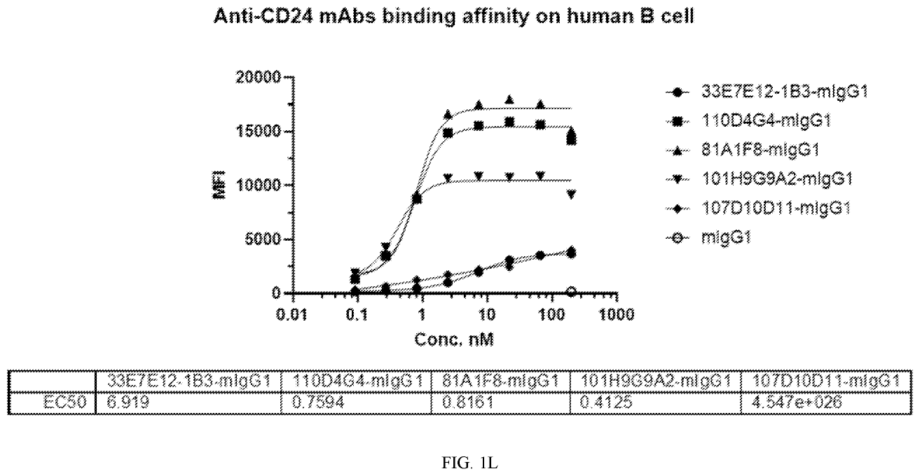

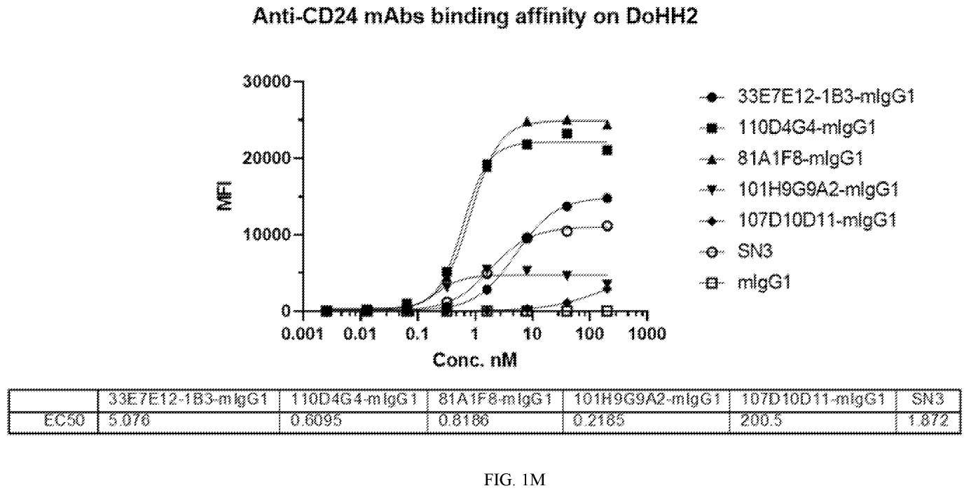

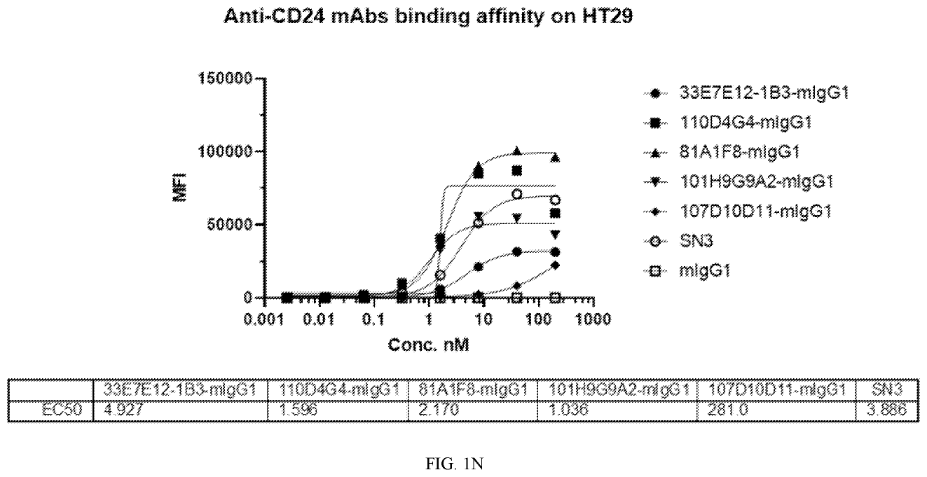

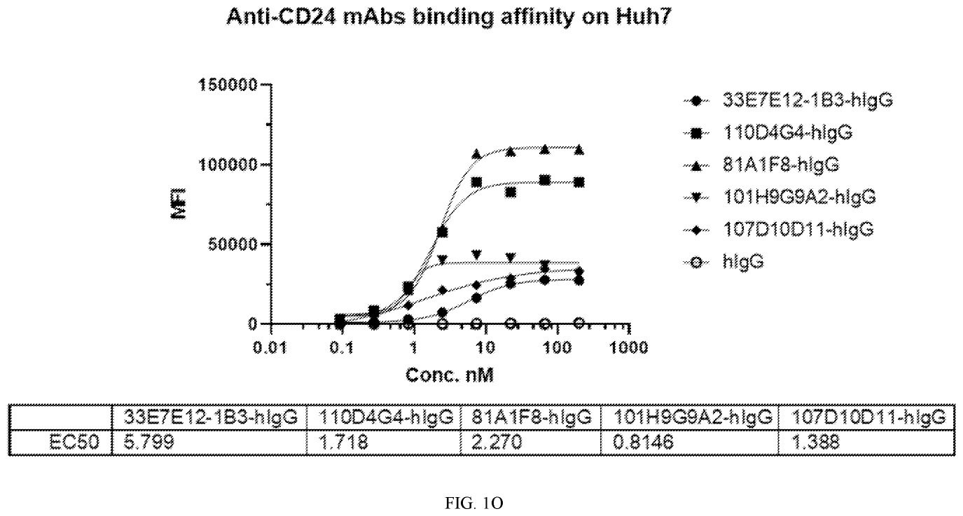

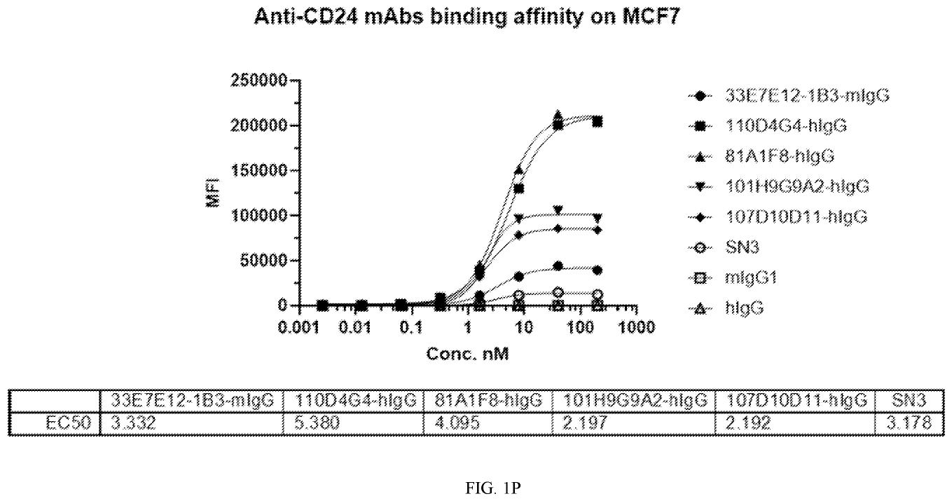

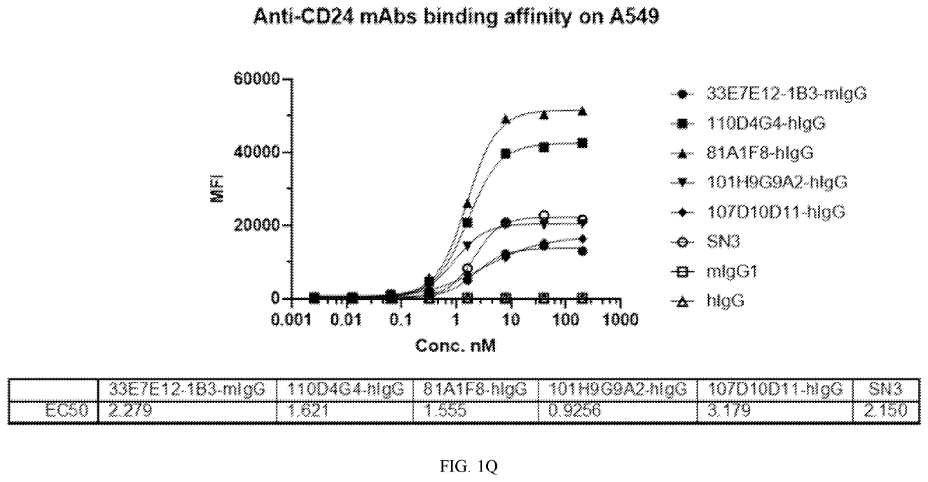

Some of our antibodies (110D4G4-hlgG4, 81A1F8-hIgG4) were more sensitive on CD24-low expressing cell MCF7 and A549 compared with SN3 (FIG. 1), with higher max MFI and higher or comparable binding affinity (Table 8A-8B, FIG. 1).

TABLE 8A

| | | | 293F- | | | | | | MDA-MB- | | | |
|---|---|---|---|---|---|---|---|---|---|---|---|---|
| | 293F blank | | hCD24 | | 5637 | | MCF7 | | 468 | | Nalm1 | |
| | Max | EC50 | Max | EC50 | Max | EC50 | Max | EC50 | Max | EC50 | Max | EC50 |
| Clone | MFI | (nM) | MFI | (nM) | MFI | (nM) | MFI | (nM) | MFI | (nM) | MFI | (nM) |
| mIgG ctrl | 176 | / | 185 | / | 185 | / | 187 | / | 190 | / | 175 | / |
| SN3 | 187 | / | 34070 | 3.25 | 9396 | 2.7 | 761 | 8.0 | 10831 | 4.2 | 48376 | 3.43 |
| 33E7E12 | 182 | / | 24235 | 2.4 | 14097 | 4.37 | 5412 | 2.38 | 10907 | 3.45 | 45524 | 6.2 |
| 71A4F12 | 499 | / | 39416 | 6.74 | 18649 | 39.66 | 12572 | 120.7 | 32523 | 11.71 | 45015 | 6.28 |
| 56A7E3 | 833 | / | 26425 | 7.52 | 18461 | 31.64 | 9191 | 81.67 | 15265 | 16.67 | 74602 | 20.92 |
| 58F10-1F6 | 774 | / | 58228 | 24.11 | 34233 | 22.38 | 17352 | 43.38 | 49793 | 38.86 | 60299 | 8.723 |
| 73H5-1G2 | 466 | / | 20125 | 16.24 | 7442 | 24.64 | 373 | 45.91 | 6404 | 27.98 | 58514 | 33.82 |
| 110D4G4 | 1225 | / | 56479 | 6.55 | 47100 | 5.889 | 21225 | 5.2 | 52775 | 12.23 | 76230 | 3.27 |
| 81A7A10 | 529 | / | 54424 | 12.14 | 23184 | 37.65 | 3838 | 61.21 | 45877 | 60.99 | 74041 | 25.75 |
| 111F3A2 | 382 | / | 58833 | 12.3 | 18221 | 10.56 | 2251 | 10.81 | 35842 | 18.79 | 73783 | 10.61 |
| 100F2E3 | 249 | / | 44060 | 11.43 | 22138 | 9.474 | 7162 | 8.08 | 43976 | 19.65 | 58827 | 8.88 |
| 81A1F8 | 698 | / | 10301 | 1.702 | 22198 | 3.31 | 11477 | 3 | 24869 | 5.23 | 24797 | 1.9 |

TABLE 8B

| | | | CHOK1- | | | | | | MDA-MB- | | | | | | 293F- | |
|---|---|---|---|---|---|---|---|---|---|---|---|---|---|---|---|---|
| | CHOK1 | | hCD24 | | MCF7 | | Nalm1 | | 468 | | 5637 | | 293F | | hCD24 | |
| | | EC50 | | EC50 | | EC50 | | EC50 | | EC50 | | EC50 | | EC50 | | EC50 |
| | MFI | (nM) | MFI | (nM) | MFI | (nM) | MFI | (nM) | MFI | (nM) | MFI | (nM) | MFI | (nM) | MFI | (nM) |
| mIgG1 | / | / | / | / | / | / | / | / | / | / | / | / | / | / | / | / |
| SN3 | / | / | 12177 | 8.67 | 570 | 6.537 | 54730 | 5.08 | 5082 | 7.371 | 3884 | 5.74 | / | / | 10513 | 3.5 |
| 107D10D11 | / | / | 18041 | 1.3 | 3814 | 25.36 | 92981 | 1.61 | 34194 | 4.294 | 17713 | 5.322 | / | / | 23562 | 1.778 |

TABLE 9

| | CHOK1-hCD24 | | HT-29 | | A549 | | H1975 | |
|---|---|---|---|---|---|---|---|---|
| | Max | EC50 | Max | EC50 | Max | EC50 | Max | EC50 |
| Clone | MFI | (nM) | MFI | (nM) | MFI | (nM) | MFI | (nM) |
| mIgG ctrl | 189 | / | 194 | / | 167 | / | 203 | / |
| SN3 | 29701 | 3.632 | 5897 | 6.614 | 8820 | 2.996 | 5206 | 8.262 |
| 33E7E12 | 24117 | 1.864 | 2453 | 3.397 | | | 6113 | 8.148 |
| 71A4F12 | 28808 | 1.715 | 4780 | 5.458 | 4409 | 1.972 | 7195 | 71.88 |
| 56A7E3 | 36721 | 4.851 | 1461 | 18.07 | 3319 | 92.42 | 2189 | 93.09 |
| 58F10-1F6 | 28095 | 9.7 | 4902 | 10.81 | 12604 | 12.64 | 17462 | 53.11 |
| 73H5-1G2 | 19885 | 23.8 | 4817 | 39.95 | 5696 | 36 | 2198 | 31.3 |
| 110D4G4 | 28908 | 1.52 | 5610 | 2.921 | 18352 | 2.719 | 24926 | 5.219 |
| 81A7A10 | 27774 | 19.36 | 5890 | 25.51 | 12218 | 11.7 | 10855 | 75.32 |
| 111F3A2 | 33600 | 8.189 | 4332 | 11.91 | 16253 | 10.05 | 10533 | 11.53 |
| 100F2E3 | 22261 | 1.706 | 2419 | 9.552 | 8667 | 6.584 | 6898 | 10.04 |
| 81A1F8 | 45923 | 1.525 | 3053 | 2.931 | 8659 | 2.374 | 10644 | 2.032 |

| | 293F blank | | 293F-hCD24 | | 5637 | | MCF7 | | MDA-MB-468 | | Nalm1 | | CHOCK1-hCD24 | |
|---|---|---|---|---|---|---|---|---|---|---|---|---|---|---|
| | Max | EC50 | Max | EC50 | Max | EC50 | Max | EC50 | Max | EC50 | Max | EC50 | Max | EC50 |
| Clone | MFI | (nM) | MFI | (nM) | MFI | (nM) | MFI | (nM) | MFI | (nM) | MFI | (nM) | MFI | (nM) |
| hIgG ctrl | | | | | | | | | | | | | | |
| ch33E7E12- | 231 | / | 80907 | 3.55 | 42895 | 6.44 | 20614 | 4.35 | 56743 | 9.07 | 108680 | 5.8 | 62147 | 2.265 |
| hIgG1 | | | | | | | | | | | | | | |
| ch33E7E12- | 130 | / | 55225 | 4.56 | 25604 | 7.27 | 16084 | 4.75 | 20088 | 6.8 | 34434 | 11.81 | 56567 | 2.664 |
| hIgG4 | | | | | | | | | | | | | | |
| ch56A7E3- | 485 | / | 57107 | 3.7 | 61080 | 138.8 | 22756 | 83.68 | 54153 | 18.73 | 80263 | 66.44 | 54119 | 3.04 |
| hIgG4 | | | | | | | | | | | | | | |
| ch110D4G4- | 1027 | / | 112190 | 2.82 | 49913 | 2.18 | 22713 | 2.01 | 89748 | 5.44 | 110671 | 1.945 | 99530 | 35.36 |
| hIgG4 | | | | | | | | | | | | | | |
| ch111F3A2- | 468 | / | 89638 | 2.94 | 21894 | 22.89 | 3649 | 92.64 | 40367 | 8.79 | 74662 | 4.22 | 88076 | 10.49 |
| hIgG4 | | | | | | | | | | | | | | |
| ch100F2E3- | 295 | / | 60388 | 3.73 | 20184 | 3.42 | 7959 | 6.85 | 71893 | 7.39 | 77702 | 2.78 | 59308 | >100 |
| hIgG4 | | | | | | | | | | | | | | |
| ch81A1F8- | 1382 | / | 116917 | 3.35 | 54063 | 2.47 | 25390 | 2.31 | 90847 | 5.75 | 101261 | 1.31 | 85348 | 3239 |
| hIgG4 | | | | | | | | | | | | | | |

Table 10 shows binding affinity of the antibodies on 293F cells, 293F-hCD24, breast cancer cells (MCF7, MDA-MB-468), bladder cancer cells (5637), chronic myelocytic leukemia cells (Nalm1), CKOK1 cells, CKOK1-hCD24 cells, colorectal cancer cells (HT29), ovarian cancer cells (SKOV3), and Lung carcinoma cells (H1975 and A549). Prefix “ch-” means chimeric, wherein hIgG1 represents heavy chain constant region of human IgG1 isotype, and hIgG4 represents heavy chain constant region of human IgG4 isotype.

FIGS. 1J-1R show binding affinity of the antibodies on acute lymphoblastic leukemia cells (Nalm6), B cell lymphoma cells (SU-DHL6 and DoHH2), human B cells, colorectal cancer cells (HT29), hepatocellular carcinoma cells (Huh7), breast cancer cells (MCF7) and lung carcinoma cells (NCI-H1975 and A549).

### Example 3. Antibody Characterization: Phagocytosis

1. Methods

Monocytes were separated from PBMC using Easysep™ human CD14 Positive selection kit II (StemCell, 17858). Monocyte-derived macrophages (MDMs) were induced by culturing the monocytes for 7 days in RPMI 1640 medium supplemented with 10% FBS and 100 ng/mL rhM-CSF; Replaced half of the medium and supply cytokines every 3 to 4 days. Add 100 ng/mL rhTGF-β and rhIL-10 and culture for additional 5 days; Replaced half of the medium and add cytokines every 3 to 4 days.

HT-29 cells (a human colorectal adenocarcinoma cell line) were labeled with fluorescence enhancing ligand (CFSE, Invitrogen, C34554) according to operational manuscript (Label 1*10\hat{ }6/ml cells with 2 μM CFSE and incubate for 20 min at 37° C. in a cell incubator). Seed 40,000 cells/well in 100 μL to 96 wells U-bottom sterile plate (Corning, cat: 3894). After that, added 50 μL serial diluted antibodies listed in Table 6 to each well and incubated the plate at 37° C. 5% C02 for 2 hr. Meanwhile, harvested MDM cells and re-suspended in RPMI1640 medium (Gibco, Cat No. #11835-030)+10% FBS to 1*10\hat{ }6/ml. Abovementioned MDM cells were disassociated by trypsin and resuspended in complete 1640 medium at 10⁶ cells/mL. 50 μL MDM (40000 cells) as mentioned were supplied into each well of the assay plate. After 2 hours incubation in 37° C., 5% C02, washed the cell mixture and stained the cells with anti-CD11b-APC for 20 min at 4° C. Washed the cells and analyzed the samples with flow cytometer.

2. Result

2.1 Anti-CD24 Antibodies could Augment the Engulfment of HT-29 Colorectal Carcinoma Cells.

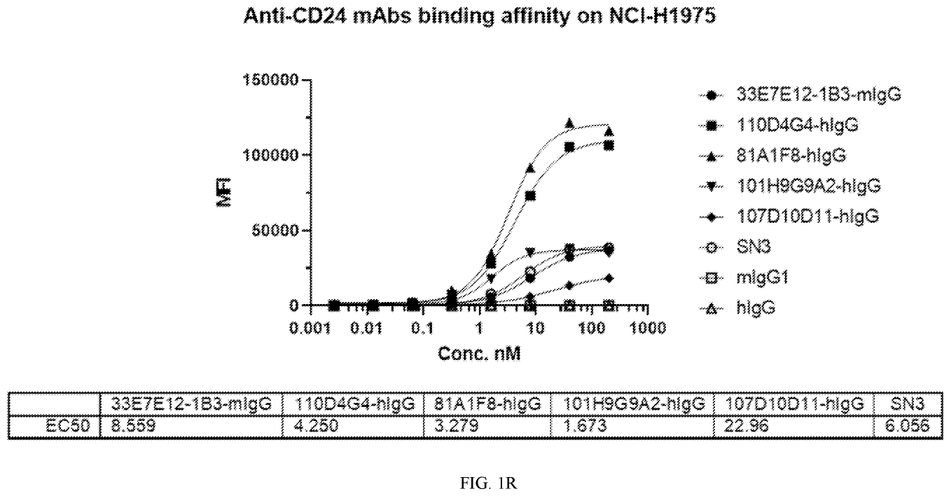

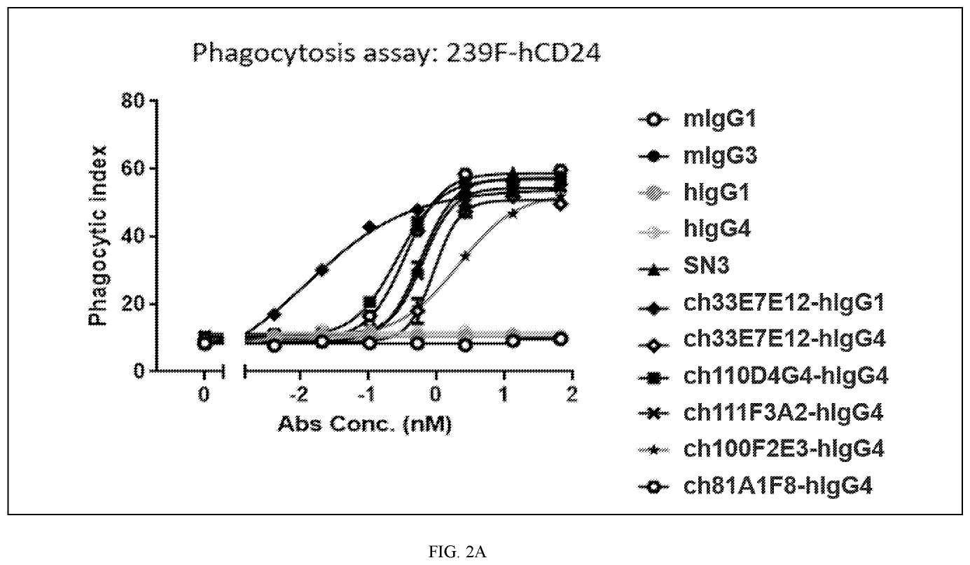

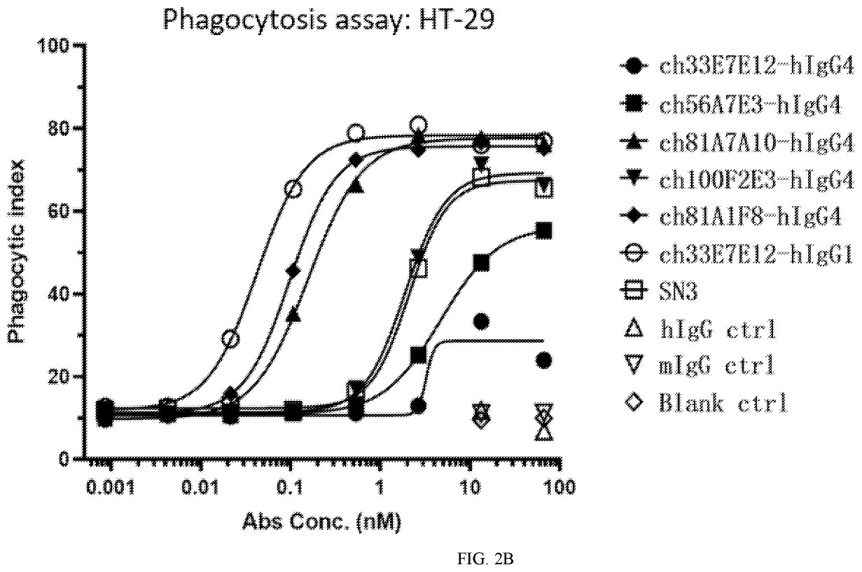

All our antibodies enhanced the phagocytosis effect on HT-29 cells. Our antibody showed higher max phagocytosis index (phagocylic index=(number of macrophages containing engulfed cells/total number of counted macrophages)×100) on HT-29 cells, and four of them showed lower EC50 compared with bench mark antibody, SN3 (Table 11, FIG. 2). The phagocytosis assay was performed following the method above-mentioned. The effector to target cell ratio (E:T) (E/T ratio) of Macrophage and tumor cells was 1:1.

TABLE 11
Max HT29 phagocytosis index and EC50 of chimeric
antibodies

| | Phagocytosis against HT-29 cells | |
|---|---|---|
| | Max phagocytosis | |
| Clone | Index (%) | EC50 (nM) |
| SN3 | 68.19 | 2.098 |
| ch33E7E12-hIgG4 | 33.30 | 3.255 |
| ch56A7E3-hIgG4 | 55.24 | 4.736 |
| ch81A7A10-hIgG4 | 78.38 | 0.157 |
| ch100F2E3-hIgG4 | 71.16 | 1.895 |
| ch81A1F8-hIgG4 | 76.63 | 0.098 |
| ch33E7E12-hIgG1 | 80.38 | 0.042 |

Some of our chimeric antibodies also enhanced the phagocytosis effect on HEK293F cells that over-express hCD24 as well as on the colorectal cancers (CRC) cell line HT-29. Some showed higher max phagocytosis index on these cells, and some showed much stronger EC50 compared with the reference antibody, SN3. hIgG1 typed chimeric antibody induced ADCP so that much better phagocytosis effect could be detected (Table 11, FIG. 2).

2.2 Combination of Anti-CD24 Antibodies with Anti-CD47 Antibodies

Combination of anti-CD47 with our anti-CD24 chimeric antibodies (chAb110D4G4, chAb111F3A2 and chAb81A1 F8) were tested. HT29 colorectal tumor cells were selected as the target cells.

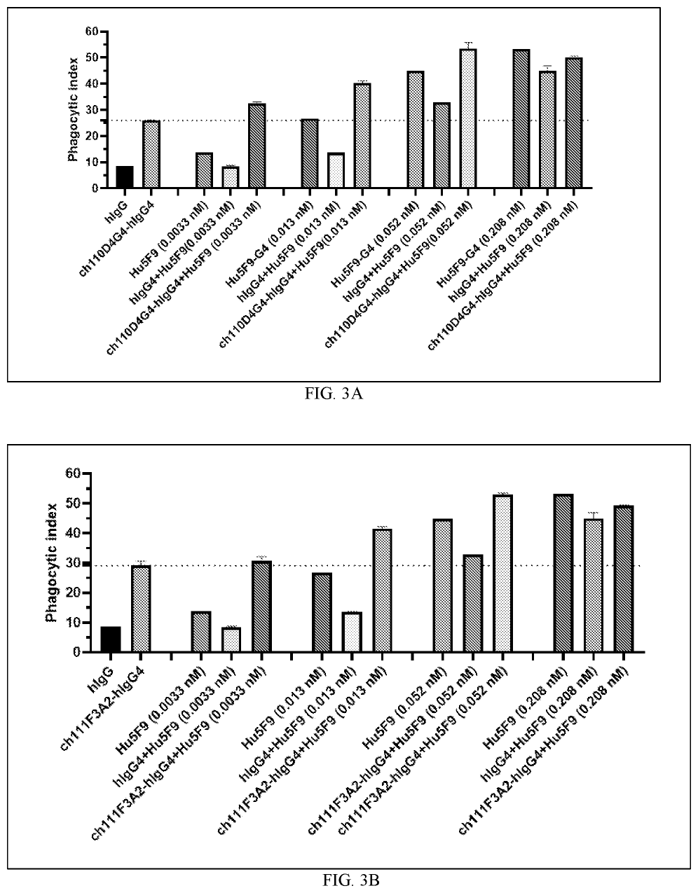

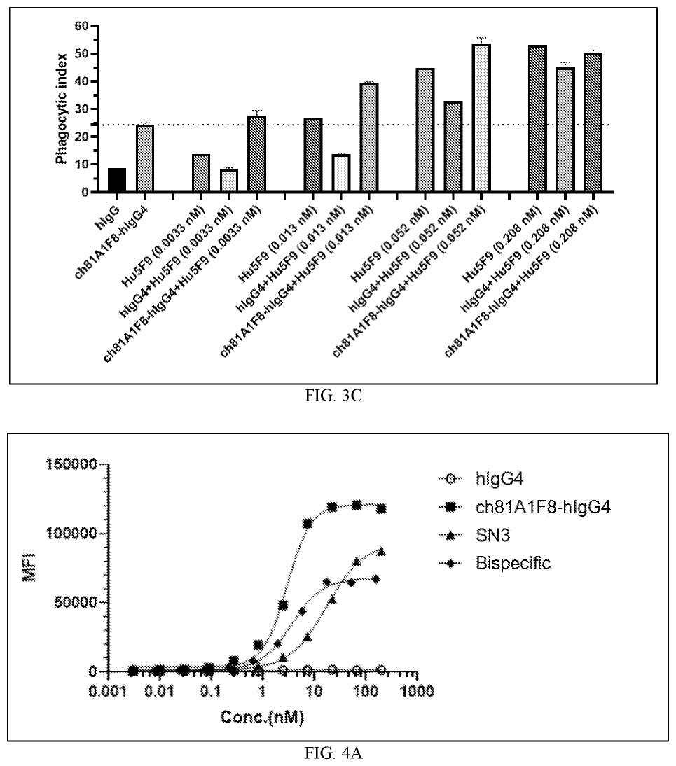

Chimeric antibody chAb110D4G4, chAb111F3A2 and chA81A1F8 were applied to combine with different concentrations of anti-CD47 antibody (hu5F9, hu5F9-hIgG4, or hu5F9-G4 are used interchangeably in FIG. 3) to detect the total phagocytosis effect. The antibody concentration of anti-CD24 chimeric antibodies was set as 0.1 nM, and hu5F9 concentration was set as 0.0033 nM, 0.013 nM, 0.052 nM and 0.208 nM. Combination of anti-CD24 and anti-CD47 improved the phagocytosis index. FIG. 3 shows that combination of an anti-CD24 antibody with an anti-CD47 antibody induced significantly better phagocytosis effect than anti-CD47 antibody alone.

2.3 Bispecific Antibody (IgG+scFv Format) was Explored for the Phagocytosis Assay.

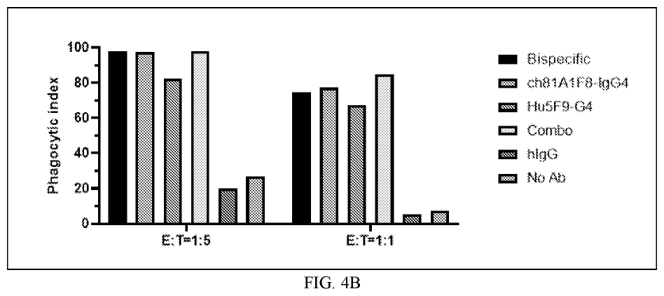

An IgG-scFv format bispecific antibody was constructed with a chAb81A1F8-IgG4 and Hu5F9 derived scFv, comprising two heavy chains in the format of VH-CH1-Hinge-CH2-CH3-spacer-scFv, associated with a light chain in the format of VL-CL, respectively. Affinity of this bispecific antibody was tested and then the phagocytosis assays was performed too (see FIG. 4). All the antibody concentration was set as 100 nM which was enough for the max phagocytosis. The E/T ratio was set as 1:5 and 1:1. Bispecific antibody showed improved phagocytosis compared with hu5F9 and the combo group showed almost 100% phagocytosis when the E/T ratio was 1:5. As shown in FIG. 4, the bispecific antibody, as well as the Combo of anti-CD24 and anti-CD47 antibodies was tested by phagocytosis assay. The max index could reach 85% when the E/T ratio was 1:1 as the assays before, and 98% with a E/T ratio of 1:5. Bispecific antibody showed very good potency of phagocytosis.

### Example 4. Antibody Humanization

Humanization of 81A1F8 and 101H9G9A2 was carried out with the following steps: 1) modelling of the mouse antibody VH and VL domains; 2) alignment with a range of preferred human germline sequences; 3) assessment of conflicts between non-human CDRs and human FRs and design of back mutations to prevent a loss of affinity in the final products; and 4) CDR grafting onto preferred germline backbones. 5 different humanized sequences were generated, followed by cloning and small-scale production of all humanized variants and chimeric in mammalian expression system.

### Example 5. Humanized Antibody Characterization: Affinity

Humanized variants 81A1F8-VH-11/VL-21(81A1F8-1121), 81A1F8-VH-21/VL-21(81A1F8-2121), 81A1F8-VH-11/VL-41(81A1F8-1141), and 81A1F8-VH-21/VL-41(81A1F8-2141) were tested via cell based binding assay using CD24 positive cells HT-29 and Nalm6. Humanized variants of 101H9G9A2 were tested via cell based binding assay using CD24 positive cells HT-29.

Briefly, HT-29 or Nalm6 cells were washed three times using PBS and firstly incubated with humanized variants. After three times washing, secondary antibody was incubated with cells. MFI of the samples was analyzed with Flow Cytometry and cells-based affinity was calculated.

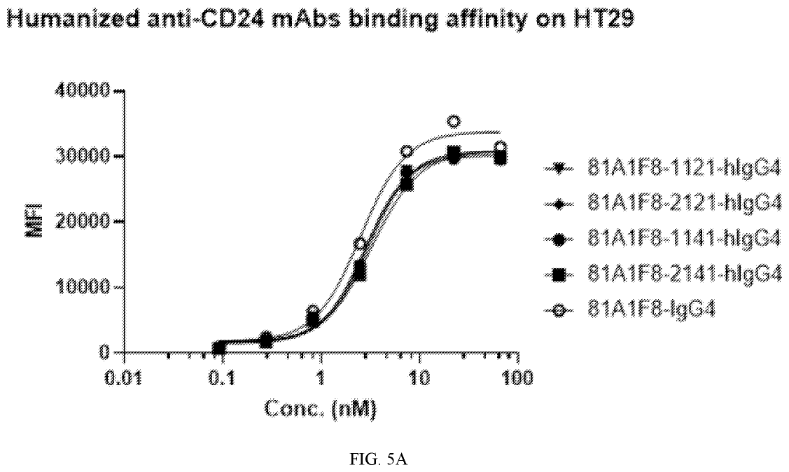

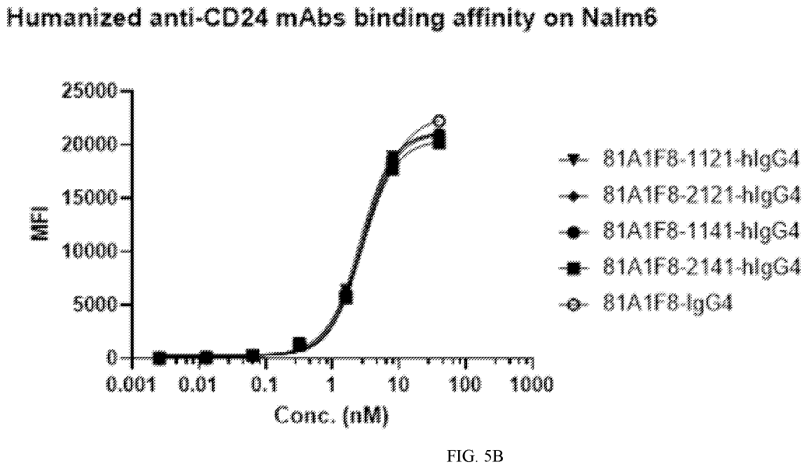

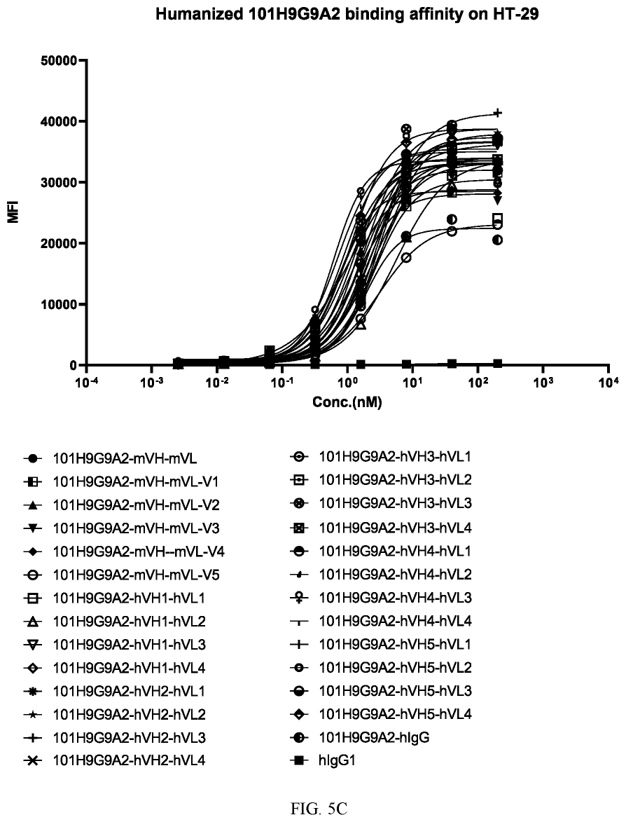

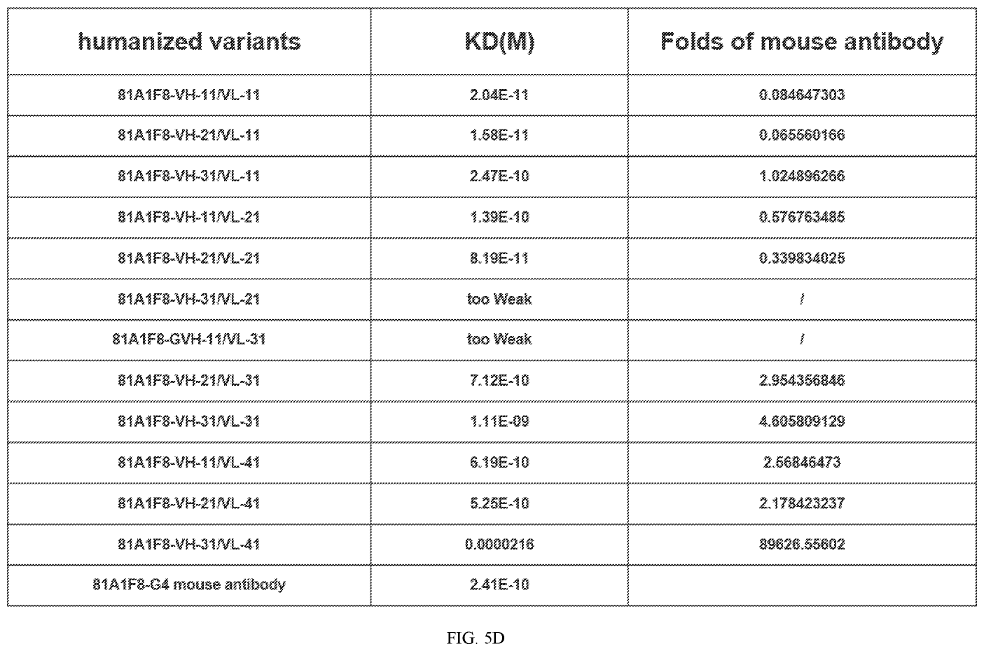

The binding affinities to HT29 and Nalm6 of the above-mentioned humanized antibodies of 81A1F8 were comparable to chimeric 81A1F8 as shown in FIG. 5A and FIG. 5B, respectively.

The binding affinities to HT29 of the above-mentioned humanized antibodies of 101H9G9A2 were shown in FIG. 5C.

Protein based KD of the humanized antibodies of 81A1F8 was tested with Gatorprime system (hCD24-Fc protein as the antigen) and the KD data was shown in FIG. 5D.

### Example 6. Glycosylation/Sialylation Epitope of CD24

The epitope pattern recognized by the antibodies provided herein was tested. Some of the antibodies provided herein were found to recognize glycosylated or sialylated modification epitope of CD24.

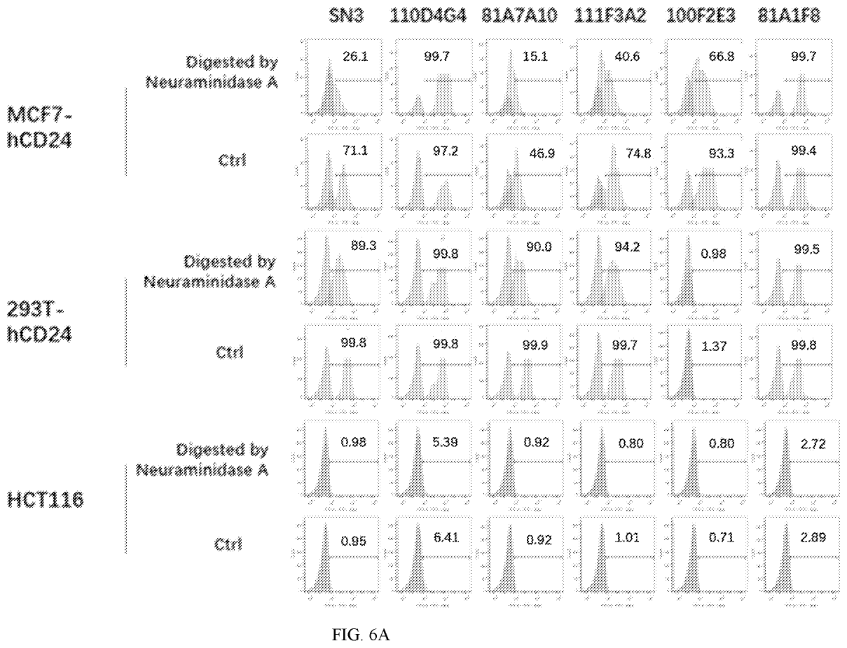

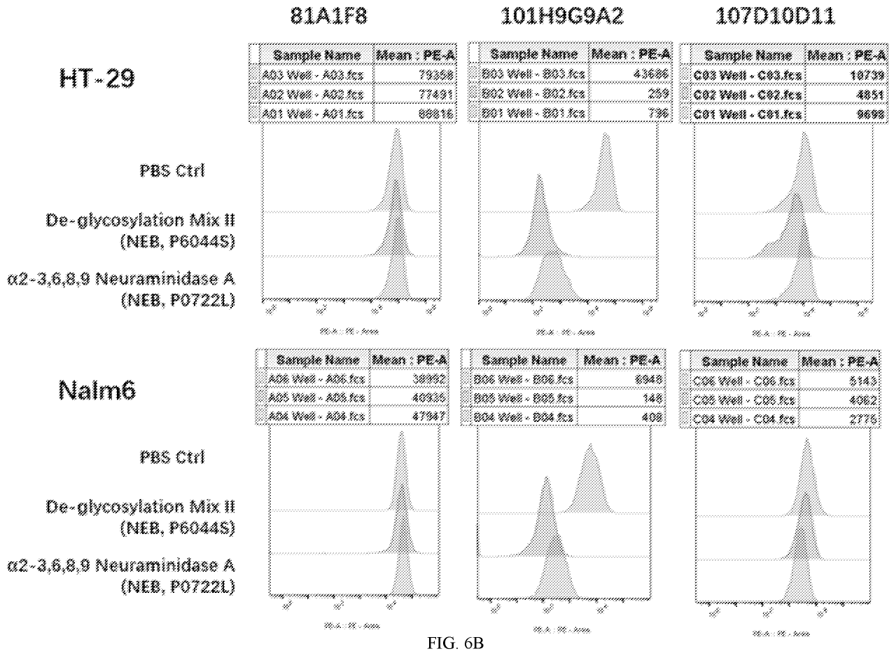

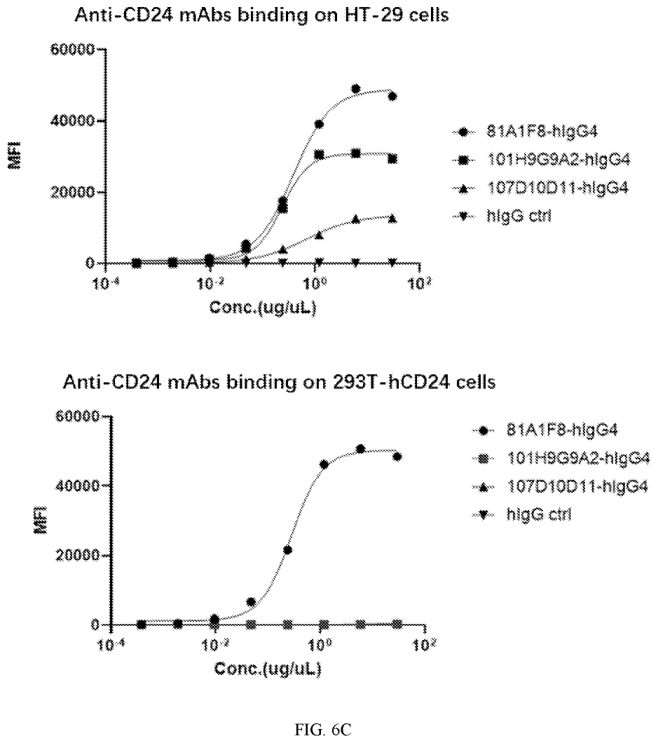

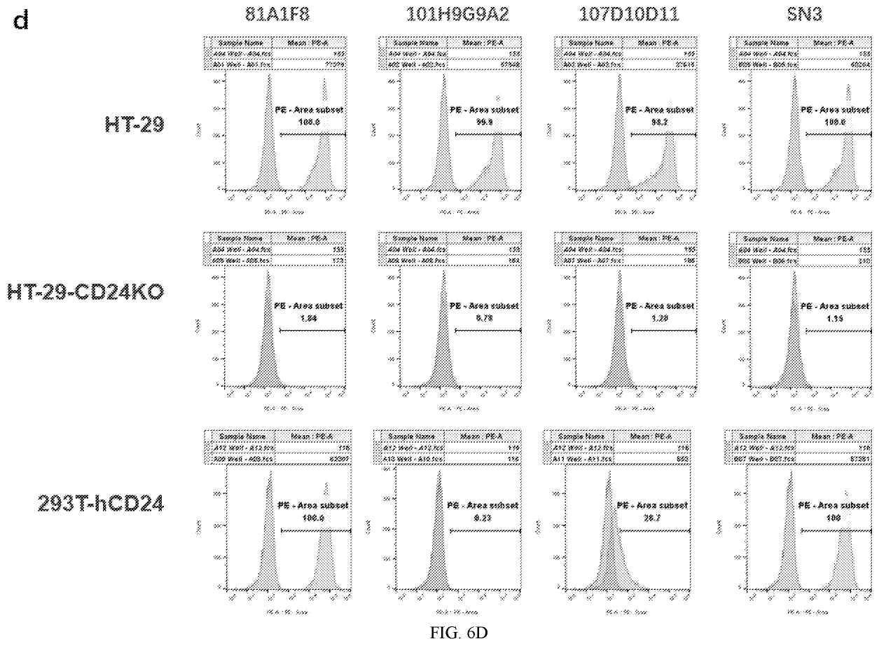

HT-29, Nalm6, HEK293T-hCD24 and MCF7-hCD24 cells were digested with neuraminidase or de-glycosylation mix kit. Compared with PBS control, 81A1F8 and 110D4G4 kept the affinity against target cells and the MFI signal did not decline. However, the MFI of 81A7A10, 111F3A2, 100F2E3, 101H9G9A2 and 107D10D11 decreased heavily after target cells treated with neuraminidase or de-glycosylation mix as shown in FIG. 6A and FIG. 6B. Bench mark antibody SN3 also recognized a glycosylation epitope which was reported before. As shown in FIG. 6C and FIG. 6D, 101H9G9A2 bound to HT-29 cells with high affinity but could not bind to 293T-hCD24 at all. 107D10D11 bound to 293T-hCD24 with low affinity. SN3, however, bound to 293T-hCD24 and HT-29 normally.

### Example 7. Affinity Test for Anti-CD24 mAbs as Well as Humanized Antibodies by SPR

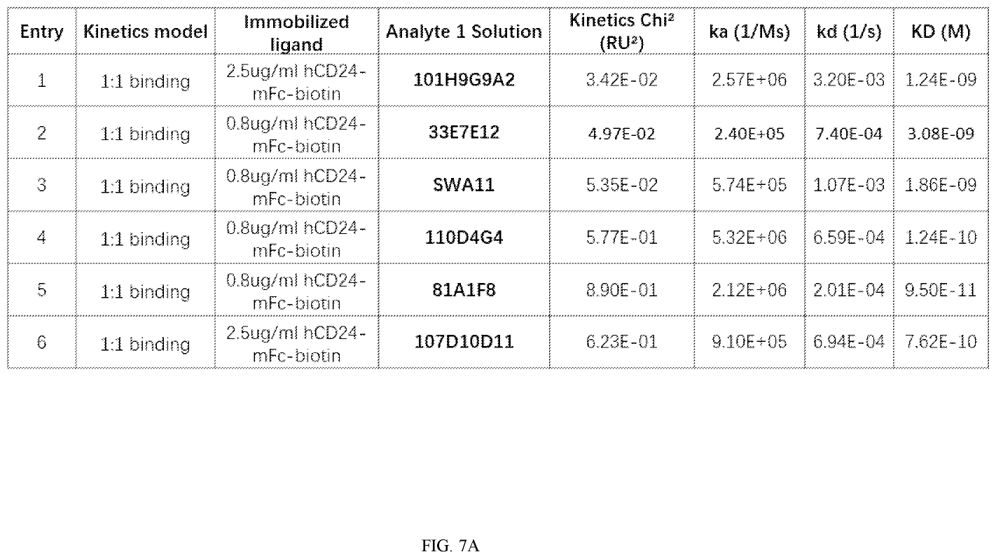

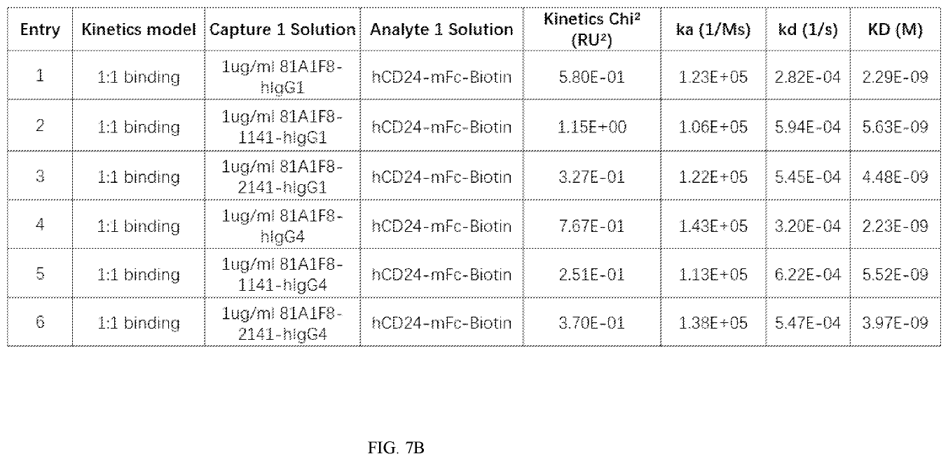

The affinity of antibodies against mFc tagged human CD24 (hCD24-mFc-biotin) was determined by BIAcore 8K (GE Healthcare). 0.8 or 2.5 μg/ml hCD24-mFc-biotin was immobilized to Series S Sensor Chip SA at a flow rate of 10 μl/min for 120 s to reach the immobilization level around 1200 RU. Antibodies were injected at a flow rate of 30 μl/min at room temperature with the concentration gradient (1.56˜50 nM). The contact time was set to 180 s and dissociation time was 400 s. At the end of each cycle, 10 mM Glycine pH1.5 was injected to remove the tested antibodies from the surface. At last, binding kinetics was calculated using BIAcore Insight Evaluation Software and a 1:1 binding model was used for curve fitting. The results of SPR analysis for some anti-CD24 mAb provided herein were displayed in FIG. 7A. The results of SPR analysis for humanized 81A1F8 candidates were displayed in FIG. 7B.

### Example 8. ELISA Based Receptor Binding Assay for 81A1F8

1. Methods

Human CD24-mFc proteins were coated on plates. Biotinylated human SigLec10 proteins (SIG-HM510B, Kactus) were mixed together with 81A1F8 of different concentrations and then added to the plates. The binding buffer that we employed was: 1% Triton-X, 0.3 mM MnCl₂, 1 mM CaCl₂, 25 mM Tris, 150 mM NaCl, 1 mM MgCl2, pH 7.6. Samples were washed three times with 1×PBS buffer. Signals of the samples were detected and normalized with original SigLec-10 readout.

2. Results

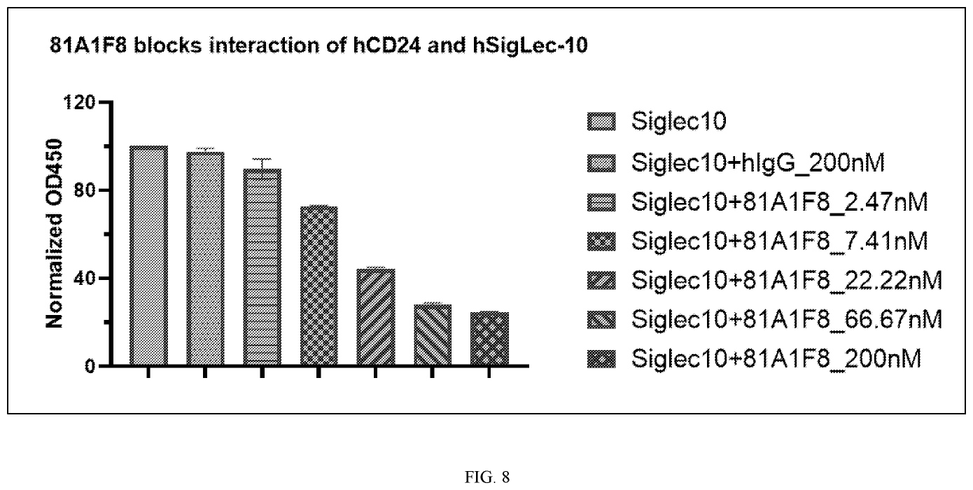

The results of ELISA based receptor binding assay for 81A1F8 of different concentrations were shown in FIG. 8.

### Example 9. In Vitro Efficacy Assays for 81A1F8 and its Humanized Variants

1. Phagocytosis Assay

Human monocytes were separated and induced as mentioned in EXAMPLE 3.

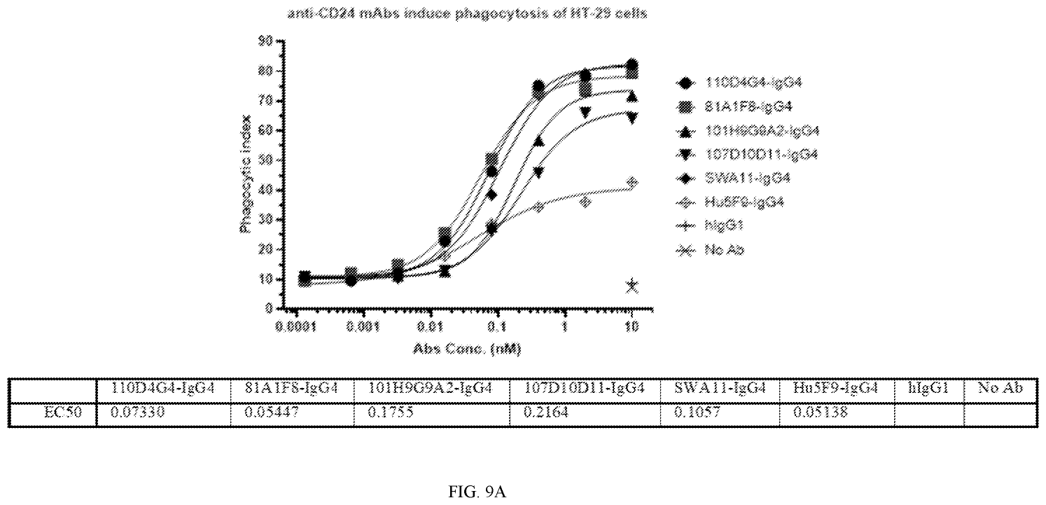

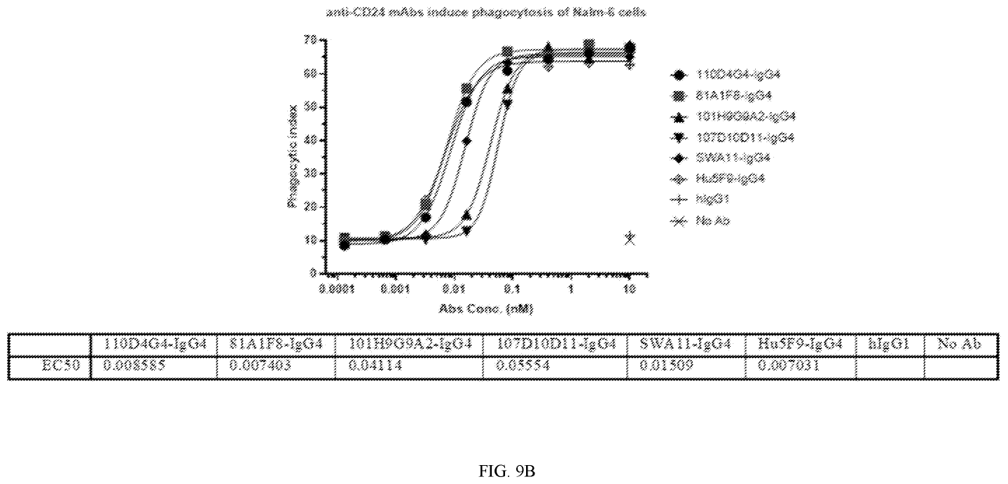

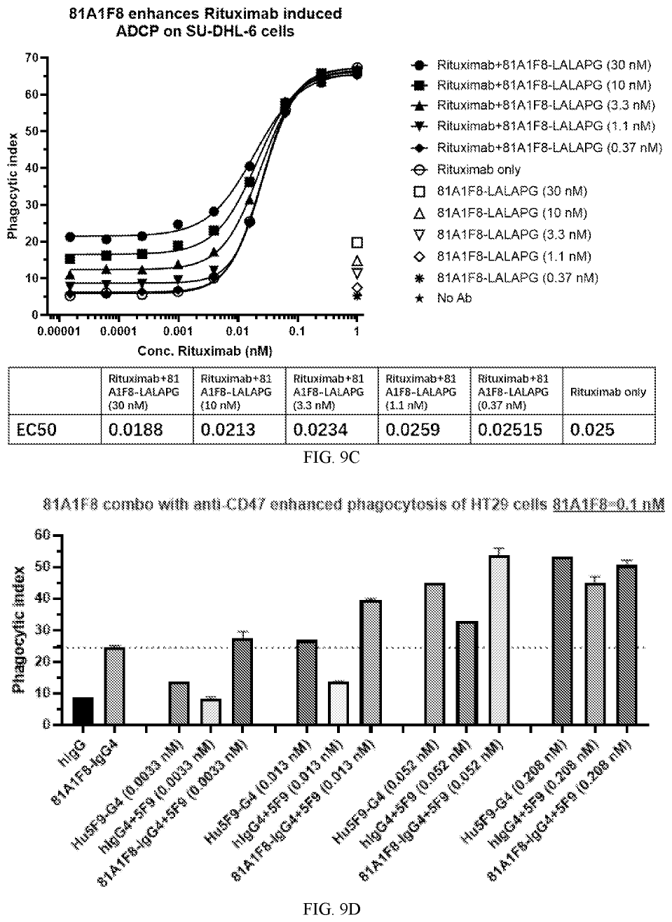

A & B: Phagocytosis of target tumor cells (HT-29 and Nalm6) was performed using anti-CD24 mAbs (hIgG4 Fc) and bench mark antibody SWA11 as well as anti-CD47 mAb 5F9-IgG4. The results were shown in FIG. 9A and FIG. 9B.

C: 81A1F8-LALAPG with rare Fc dependent functions, was combined with Rituximab to see if it could enhance the ADCP effect of Rituximab. We found that additive effect could be detected (see FIG. 9C).

D: 81A1F8-hIgG4 chimeric antibody also enhanced the phagocytosis effect of anti-CD47 antibody hu5F9 when administered in combination (see FIG. 9D).

2. ADCC and CDC Assays

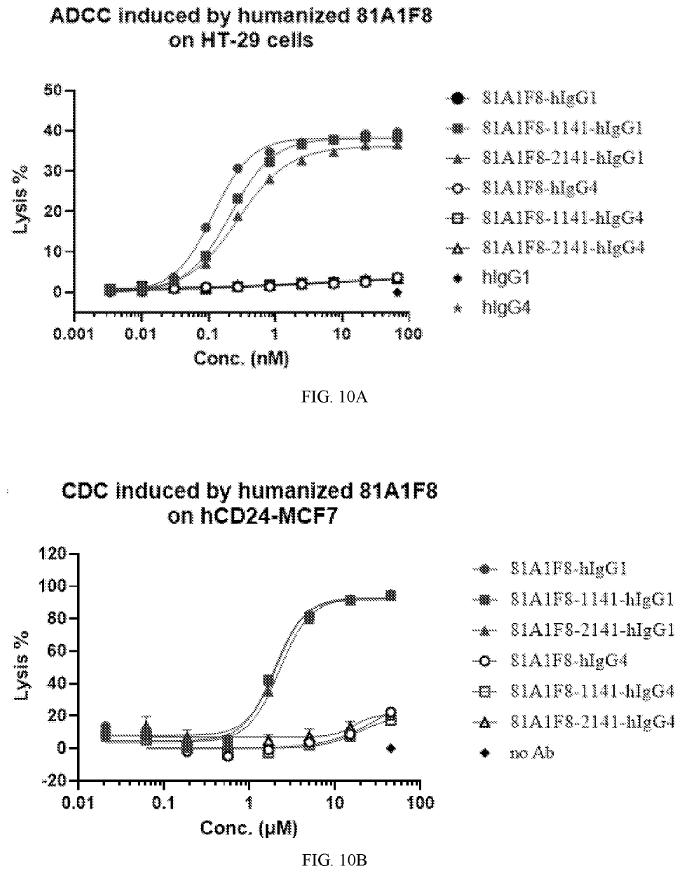

A: ADCC effects of 81A1F8 and its humanized variants were tested on target cell HT-29. HT-29 cells were incubated with PBMC. Different concentrations of 81A1F8 as well as humanized variants 81A1F8-1141 and 81A1F8-2141 were added to samples. Lysis of target cells were tested via LDH kit. As shown in FIG. 10A, ADCC effect could only be induced by hIgG1 iso-typed antibodies.

B: CDC effects of 81A1F8 and its humanized variants was tested on target cell hCD24-MCF7. Different concentrations of antibodies were incubated with target cells and complement system. As shown in FIG. 10B, over 90% lysis of target cells could be detected at high concentrations of 81 A1F8 antibody variants.

### Example 10. In Vivo Efficacy Assays of Anti-CD24 Antibodies

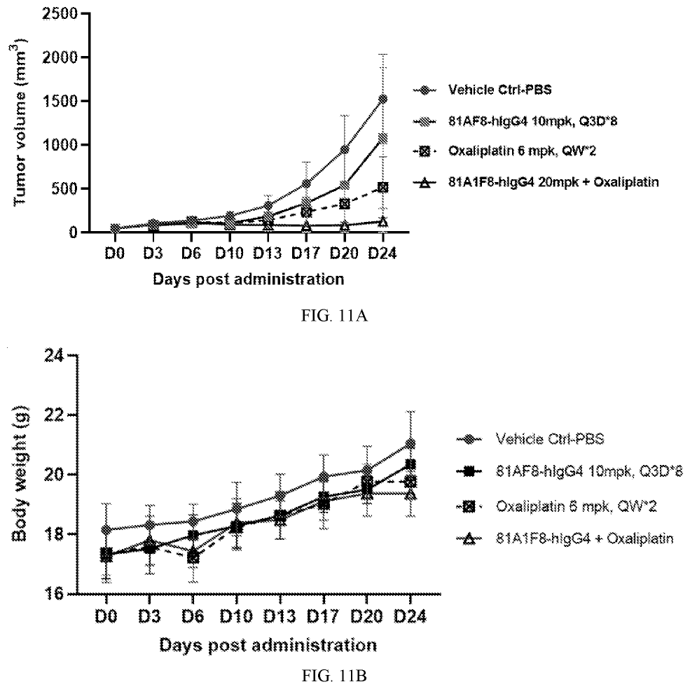

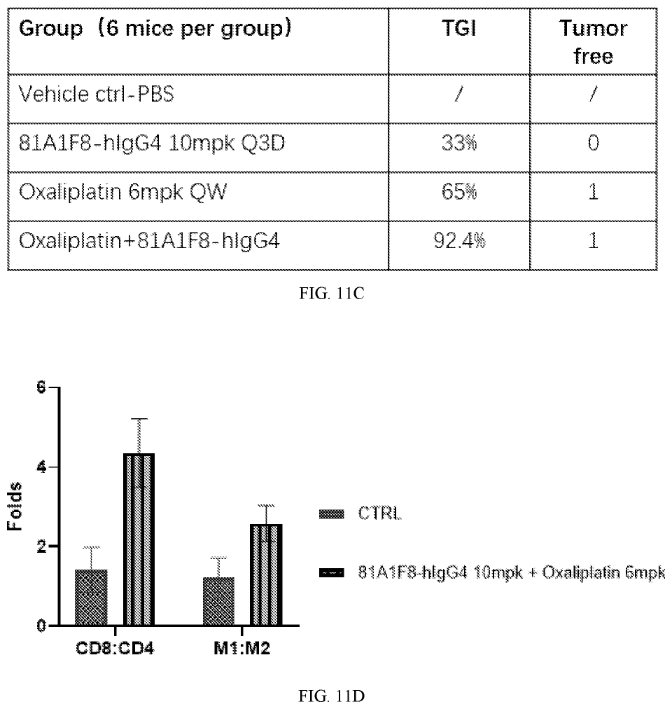

In vivo efficacy of 81A1F8 was tested in C57BL/6J syngeneic mice model. Briefly, 1 million MC38-hCD24 cells were implanted subcutaneously to female, 6-8 weeks C57BL/6J mice. When average tumor volume reached ˜60 mm³, mice were randomly grouped and treated as shown below. hIgG4 iso-typed antibody was tested in single agent (10 mpk, Q3D) or combo with Oxaliplatin (6 mpk, QW). Tumor volume and mice body weight were measured twice a week. In vivo efficacy of 81A1F8 was displayed in FIG. 11A-FIG. 11D.

All the treated groups showed tumor growth inhibition (see FIG. 11A). When combo with Oxaliplatin, 81A1F8-hIgG4 showed much better efficacy and synergistic effect could be observed; body weight of all mice did not decline (see FIG. 11B). Summary of the in vivo efficacy test was shown in FIG. 11C. Tumor infiltrating lymphocytes were isolated from tumor tissue and analyzed via flow cytometry. FIG. 11D showed CD8/CD4 ratio, as well as M1/M2 macrophage ratio increased a lot when treated with the combination of 81A1F8-hIgG4 and Oxaliplatin, as compared to control (CTRL).

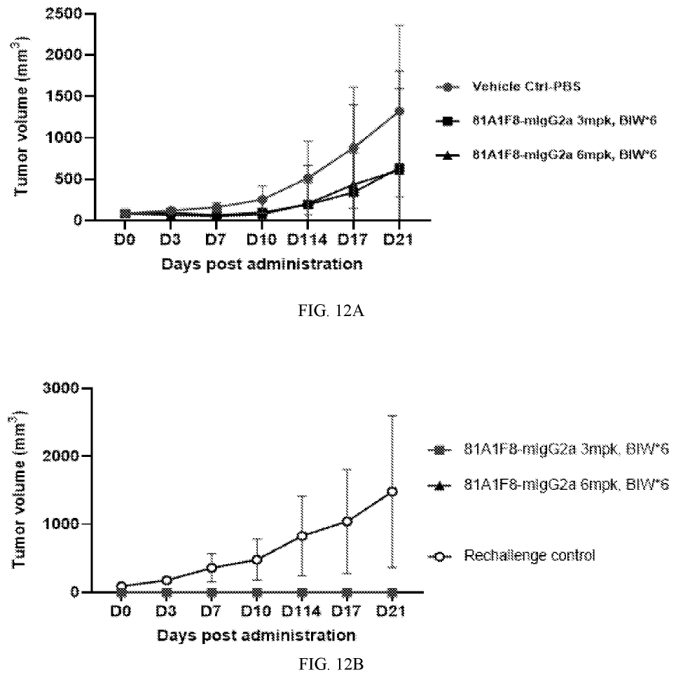

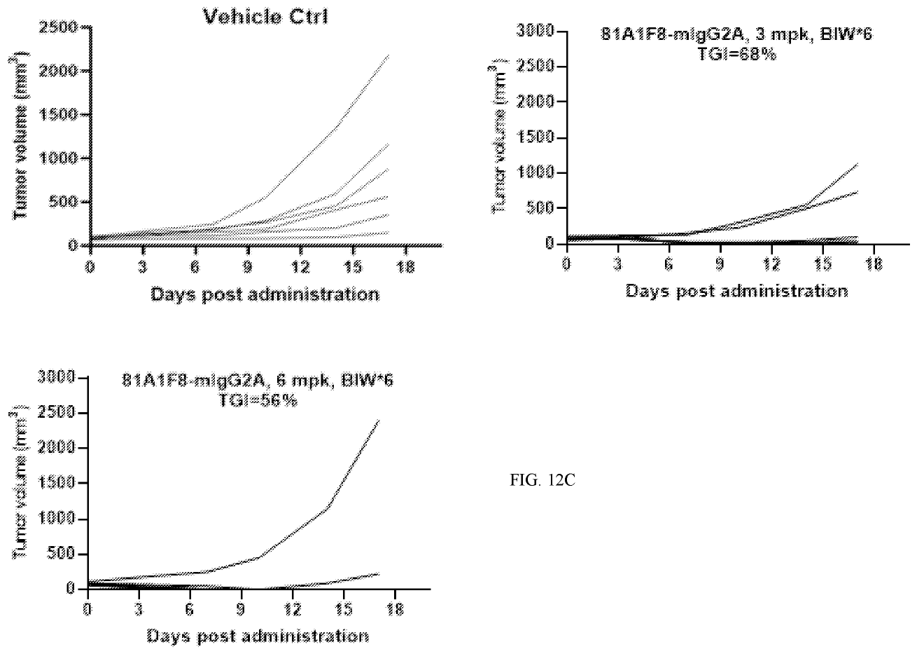

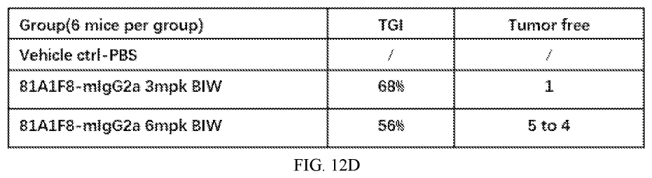

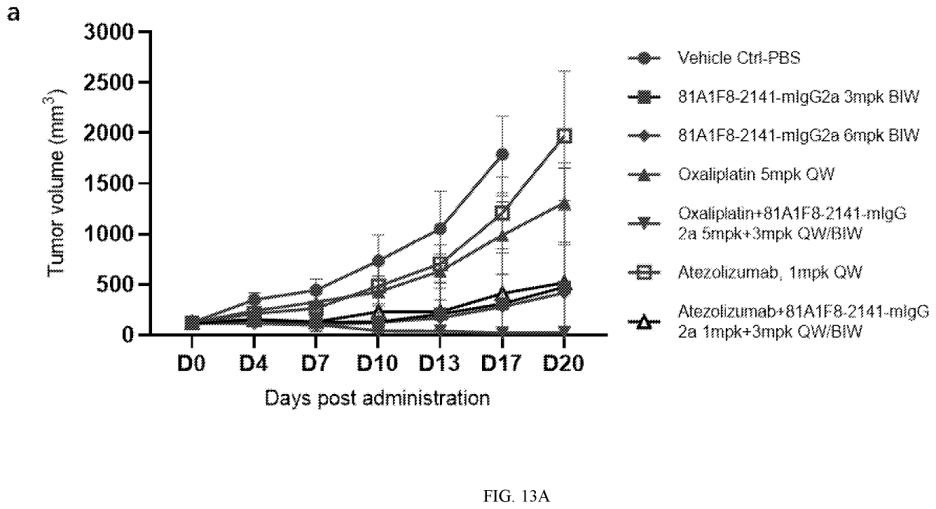

In vivo efficacy of 81A1F8-mIgG2a (homolog of human IgG1) on MC38-hCD24 syngeneic model was evaluated too. A dosage-efficacy test was performed and we found that 6 mpk dosage showed best efficacy since 4 out of 6 mice were tumor free after the administration of 81A1F8-mIgG2a (see FIG. 12A).

A rechallenge assay was performed by inoculating MC38-Hcd24 cells again and tumors could grow up. However, no tumors relapsed again in treatment groups, showing that immune memory formed after 81A1F8 treatment (see FIG. 12B).

FIG. 12C showed tumor growth curves for each mouse treated with vehicle control or 81 A1F8-mIgG2a.

FIG. 12D showed summary for the in vivo efficacy of 81A1F8-mIgG2a (also named “ATG031-mAb011-mIgG2a”).

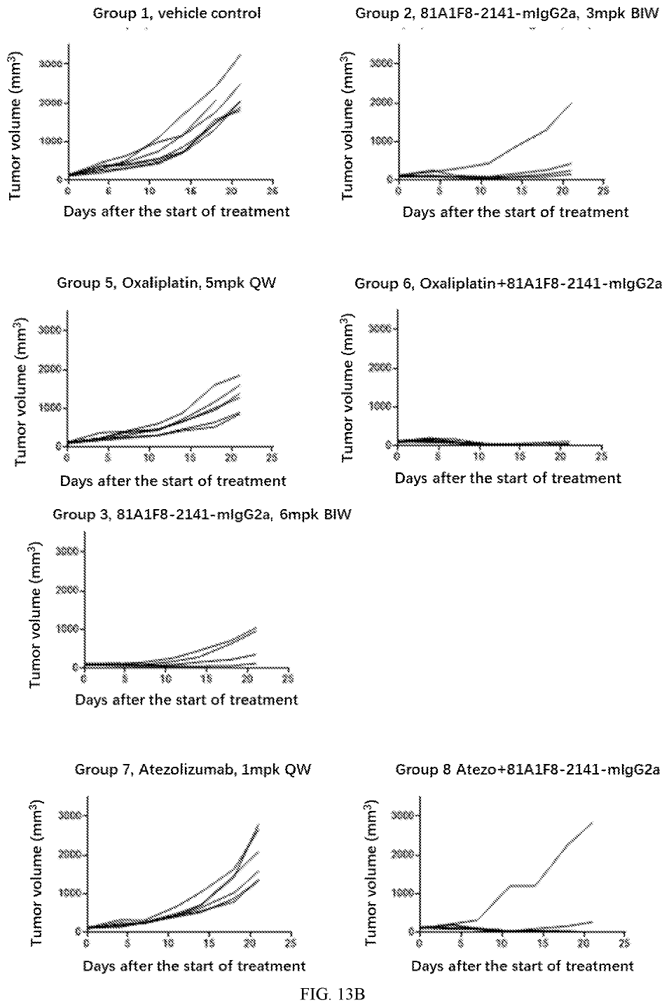

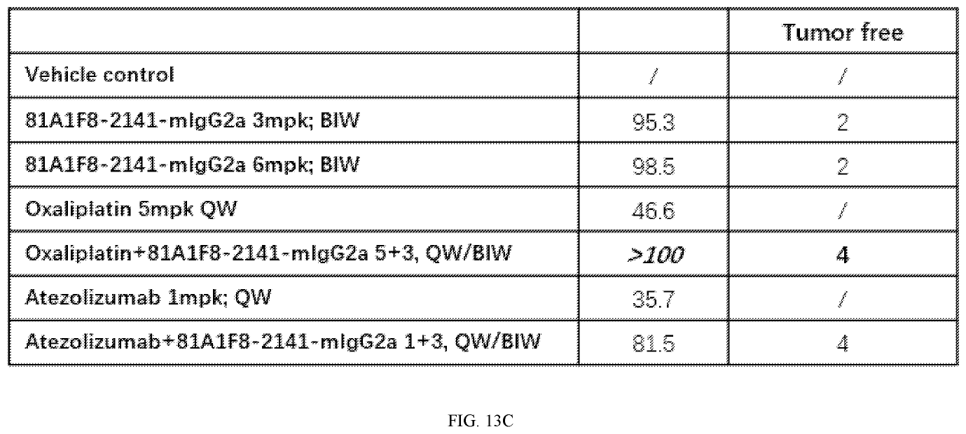

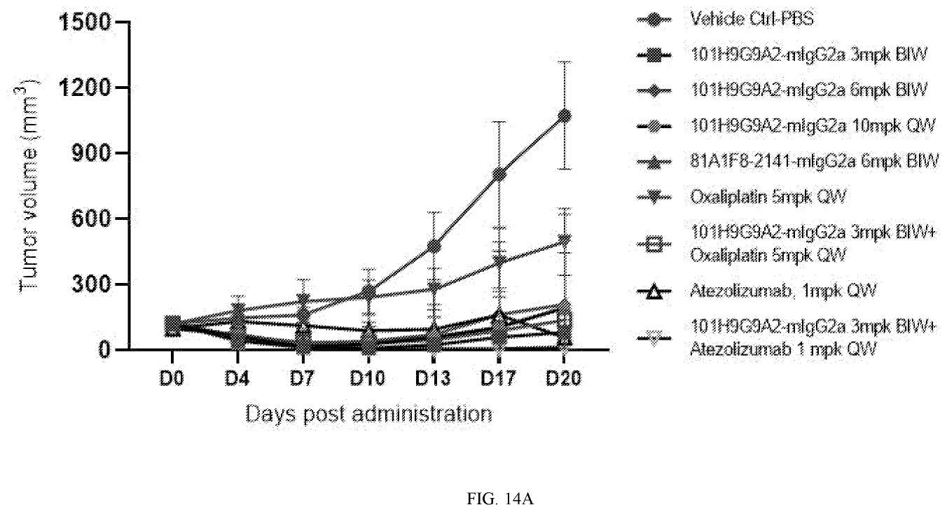

In vivo efficacy for humanized 81A1F8 (81A1F8-2141-mIgG2a) was also tested. Briefly, MC38-hCD24 tumor cells were inoculated. When average tumor volume reached ˜120 mm³, mice were grouped and treated with single agent (81A1F8-2141-mIgG2a, Oxaliplatin or Atezolizumab) or combination strategy. The tumor inhibitory effect of the tested humanized 81A1F8 (81A1F8-2141-mIgG2a) was shown in FIG. 13A-FIG. 13C, wherein FIG. 13A showed the tumor growth curve after the administration of drugs, FIG. 13B showed the tumor growth curve for each mouse of the treatment group, and FIG. 13C showed the Summary for the in vivo efficacy of 81A1F8-2141-mIgG2a.

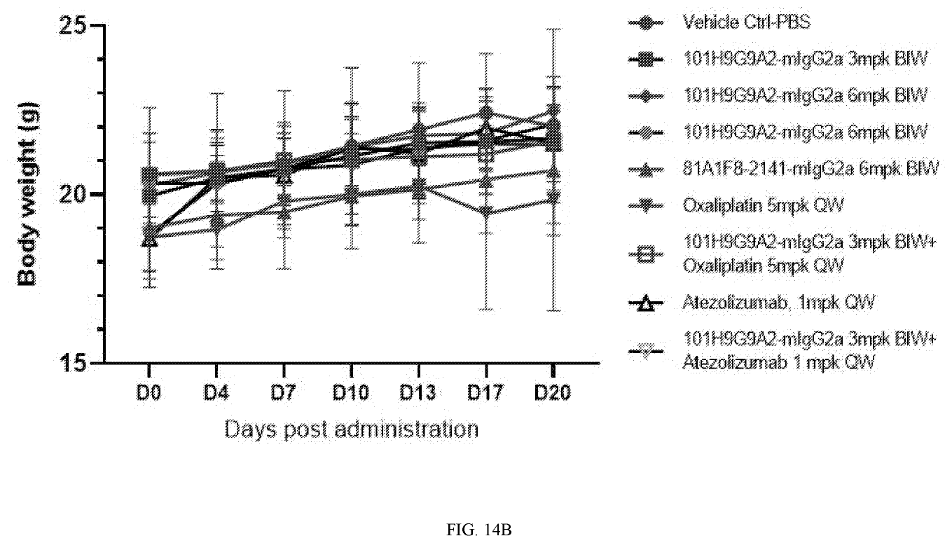

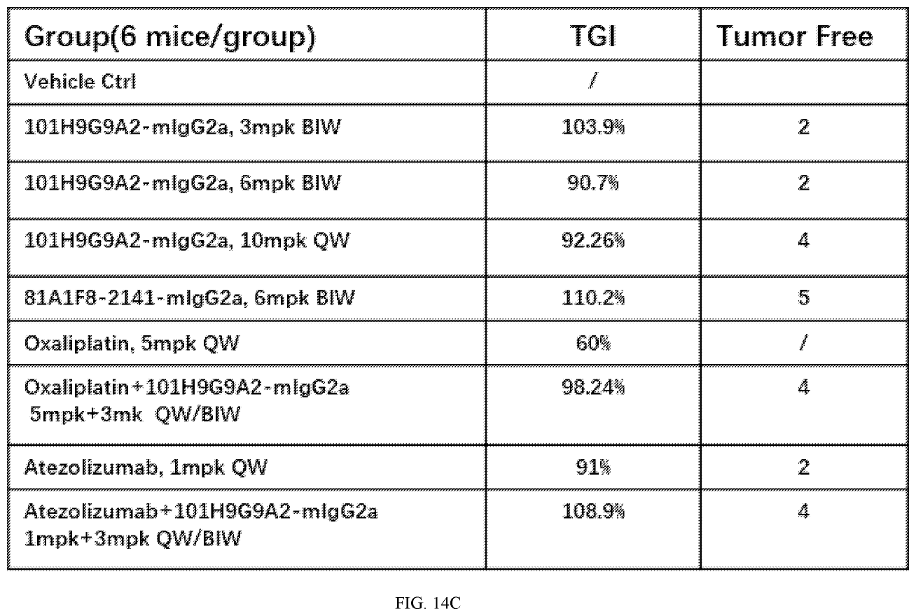

In vivo efficacy assay for 101H9G9A2-mIgG2a was also tested. Briefly, MC38-hCD24 tumor cells were inoculated as abovementioned. 101H9G9A2-mIgG2a, 81A1F8-2141-mIgG2a, Oxaliplatin and Atezolizumab or combination thereof were administrated to the mice when average tumor volume reached ˜120 mm³. The tumor inhibitory effect of the tested 101H9G9A2-mIgG2a and humanized 81A1F8 (81A1F8-2141-migG2a) was shown in FIG. 14A-FIG. 14C, wherein FIG. 14A showed the tumor volume curve of the mice that were grouped as shown, FIG. 14B showed the body weight of the mice, and FIG. 14C showed the summary of the efficacy of different treatment.

### Example 11. Cynomolgus Monkey (Macaca fascicularis) CD24 (cmCD24) Antibody Discovery

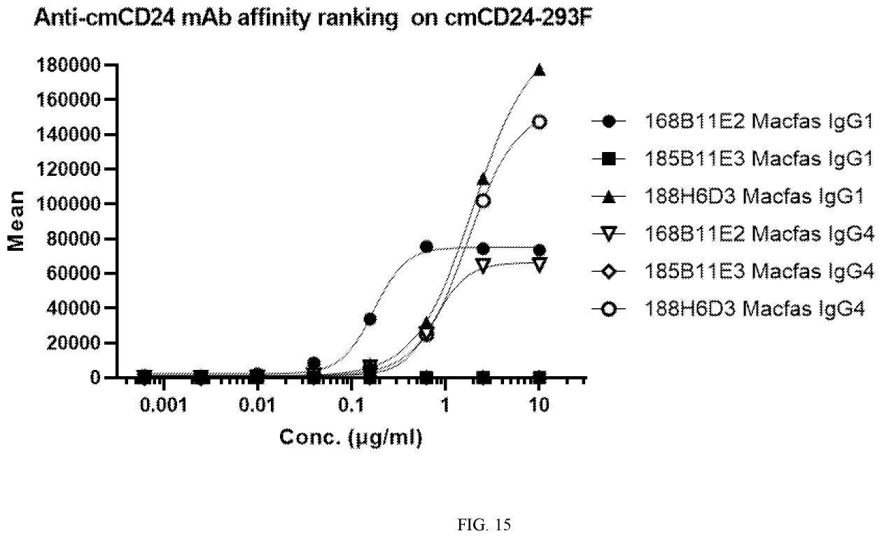

Balb/c and SJL mice were immunized with 293F cells that over expressed cmCD24-Flag and two antibodies were identified to recognize cynomolgus monkey CD24. Cell based binding data were shown in FIG. 15.

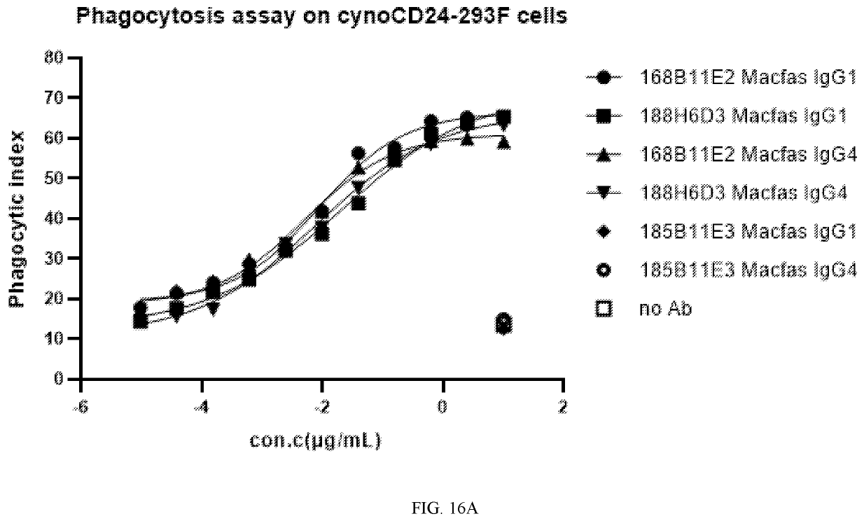

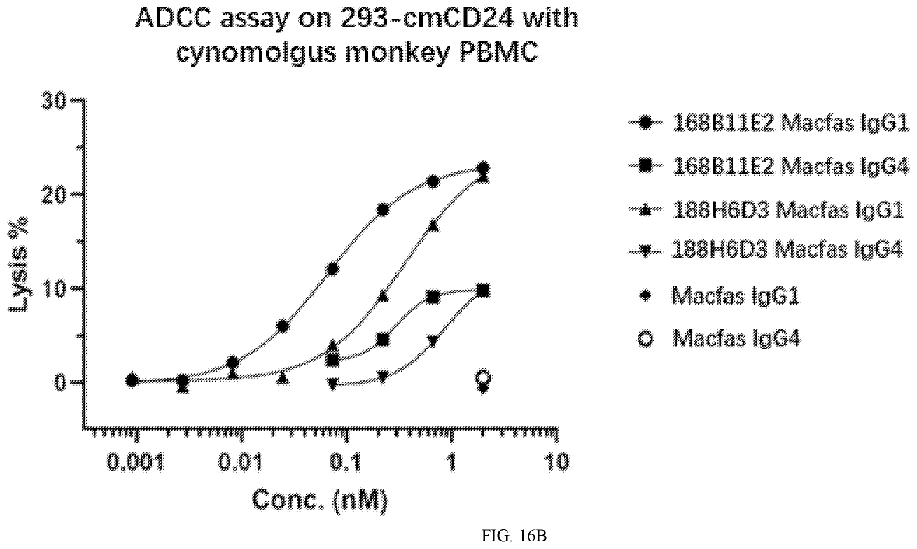

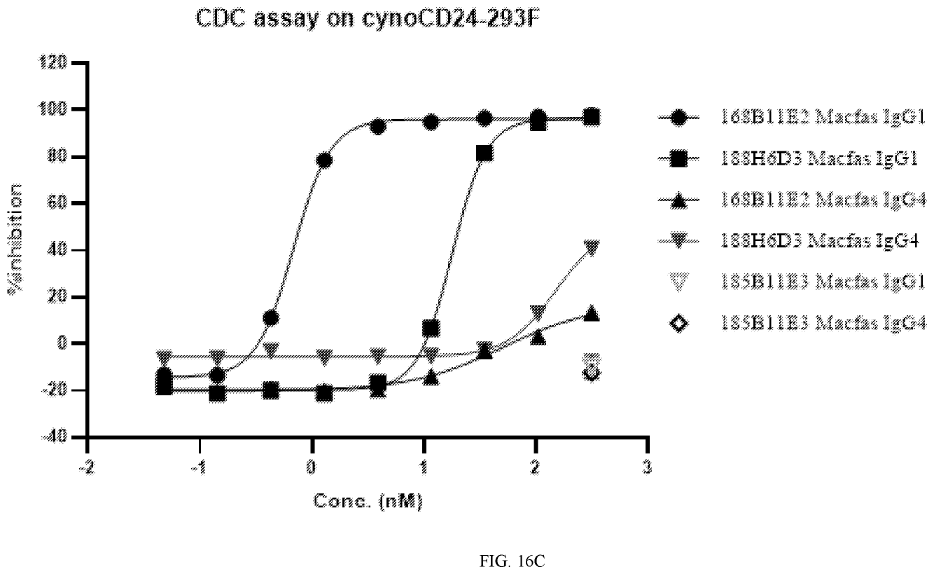

In vitro efficacy study of anti-cmCD24 antibodies was performed, and results from which was shown in FIG. 16A-FIG. 16C, wherein FIG. 16A showed the results from the phagocytosis assay of anti-cmCD24 antibodies provided herein, FIG. 16B showed the ADCC effect of the anti-cmCD24 antibodies provided herein, and FIG. 16C: showed the CDC effect of the anti-cmCD24 antibodies provided herein.

1-76. (canceled)

77. An isolated antibody or an antigen-binding fragment thereof, comprising:
a) a heavy chain variable region comprising 1, 2, or 3 CDR sequences selected from SEQ ID NO: 1, SEQ ID NO: 3, and SEQ ID NO: 5; and a light chain variable region comprising 1, 2, or 3 CDR sequences selected from SEQ ID NO: 2, SEQ ID NO: 4, and SEQ ID NO: 6;
b) a heavy chain variable region comprising 1, 2, or 3 CDR sequences selected from SEQ ID NO: 7, SEQ ID NO: 9, and SEQ ID NO: 11; and a light chain variable region comprising 1, 2, or 3 CDR sequences selected from SEQ ID NO: 8, SEQ ID NO: 10, and SEQ ID NO: 12;
c) a heavy chain variable region comprising 1, 2, or 3 CDR sequences selected from SEQ ID NO: 7, SEQ ID NO: 14, and SEQ ID NO: 11; and a light chain variable region comprising 1, 2, or 3 CDR sequences selected from SEQ ID NO: 13, SEQ ID NO: 10 and SEQ ID NO: 15;
d) a heavy chain variable region comprising 1, 2, or 3 CDR sequences selected from SEQ ID NO: 16, SEQ ID NO: 17, and SEQ ID NO: 18; and
a light chain variable region comprising 1, 2, or 3 CDR sequences selected from SEQ ID NO: 13, SEQ ID NO: 10, and SEQ ID NO: 15;
e) a heavy chain variable region comprising 1, 2, or 3 CDR sequences selected from SEQ ID NO: 19, SEQ ID NO: 21, and SEQ ID NO: 23; and
a light chain variable region comprising 1, 2, or 3 CDR sequences selected from SEQ ID NO: 20, SEQ ID NO: 22, and SEQ ID NO: 24;
f) a heavy chain variable region comprising 1, 2, or 3 CDR sequences selected from SEQ ID NO:25, SEQ ID NO: 27, and SEQ ID NO: 28; and a light chain variable region comprising 1, 2, or 3 CDR sequences selected from SEQ ID NO: 26, SEQ ID NO: 22, and SEQ ID NO:24;
g) a heavy chain variable region comprising 1, 2, or 3 CDR sequences selected from SEQ ID NO:25, SEQ ID NO: 30, and SEQ ID NO: 28; and a light chain variable region comprising 1, 2, or 3 CDR sequences selected from SEQ ID NO: 29, SEQ ID NO: 22, and SEQ ID NO: 31;
h) a heavy chain variable region comprising 1, 2, or 3 CDR sequences selected from SEQ ID NO: 32, SEQ ID NO: 34, and SEQ ID NO: 36; and
a light chain variable region comprising 1, 2, or 3 CDR sequences selected from SEQ ID SEQ ID NO: 33, SEQ ID NO: 35, and SEQ ID NO: 37;
i) a heavy chain variable region comprising 1, 2, or 3 CDR sequences selected from SEQ ID NO: 38, SEQ ID NO: 40, and SEQ ID NO: 42; and
a kappa light chain variable region comprising 1, 2, or 3 CDR sequences selected from SEQ ID NO: 39, SEQ ID NO: 41, and SEQ ID NO: 43;
j) a heavy chain variable region comprising 1, 2, or 3 CDR sequences selected from SEQ ID NO: 44, SEQ ID NO: 46, and SEQ ID NO: 48; and
a kappa light chain variable region comprising 1, 2, or 3 CDR sequences selected from SEQ ID NO: 45, SEQ ID NO: 47, and SEQ ID NO: 49;
k) a heavy chain variable region comprising 1, 2, or 3 CDR sequences selected from SEQ ID NO: 95, SEQ ID NO: 96, and SEQ ID NO: 97; and
a kappa light chain variable region comprising 1, 2, or 3 CDR sequences selected from SEQ ID NO: 98, SEQ ID NO: 99, and SEQ ID NO: 100;
l) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 107, SEQ ID NO: 108, and SEQ ID NO: 37;
m) a heavy chain variable region comprising 1, 2, or 3 CDR sequences selected from SEQ ID NO: 113, SEQ ID NO: 114, and SEQ ID NO: 115;
and a kappa light chain variable region comprising 1, 2, or 3 CDR sequences selected from SEQ ID NO: 13, SEQ ID NO: 116, and SEQ ID NO: 117;
n) a heavy chain variable region comprising 1, 2, or 3 CDR sequences selected from SEQ ID NO: 126, SEQ ID NO: 127, and SEQ ID NO: 128;
and a kappa light chain variable region comprising 1, 2, or 3 CDR sequences selected from SEQ ID NO: 129, SEQ ID NO: 130, and SEQ ID NO: 131;
o) a heavy chain variable region comprising 1, 2, or 3 CDR sequences selected from SEQ ID NO: 25, SEQ ID NO: 136, and SEQ ID NO: 137;
and a kappa light chain variable region comprising 1, 2, or 3 CDR sequences selected from SEQ ID NO: 138, SEQ ID NO: 139, and SEQ ID NO: 140;
p) a heavy chain variable region comprising 1, 2, or 3 CDR sequences selected from SEQ ID NO: 25, SEQ ID NO: 30, and SEQ ID NO: 28; and
a kappa light chain variable region comprising 1, 2, or 3 CDR sequences selected from SEQ ID NO: 29, SEQ ID NO: 145, and SEQ ID NO: 24;
q) a heavy chain variable region comprising 1, 2, or 3 CDR sequences selected from SEQ ID NO: 150, SEQ ID NO: 151, and SEQ ID NO: 152;
and a kappa light chain variable region comprising 1, 2, or 3 CDR sequences selected from SEQ ID NO: 153, SEQ ID NO: 154, and SEQ ID NO: 155;
r) a heavy chain variable region comprising 1, 2, or 3 CDR sequences selected from SEQ ID NO: 160, SEQ ID NO: 161, and SEQ ID NO: 162;
and a kappa light chain variable region comprising 1, 2, or 3 CDR sequences selected from SEQ ID NO: 163, SEQ ID NO: 154, and SEQ ID NO: 155;
s) a heavy chain variable region comprising 1, 2, or 3 CDR sequences selected from SEQ ID NO: 168, SEQ ID NO: 169, and SEQ ID NO: 170;
and a kappa light chain variable region comprising 1, 2, or 3 CDR sequences selected from SEQ ID NO: 171, SEQ ID NO: 172, and SEQ ID NO: 173;
t) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 184, SEQ ID NO: 108, and SEQ ID NO: 37;
u) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 185, SEQ ID NO: 108, and SEQ ID NO: 37;
v) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 186, SEQ ID NO: 108, and SEQ ID NO: 37;
w) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 187, SEQ ID NO: 108, and SEQ ID NO: 37;
x) a heavy chain variable region comprising 1, 2, or 3 CDR sequences selected from SEQ ID NO: 104, SEQ ID NO: 105, and SEQ ID NO: 106;
and a kappa light chain variable region comprising 1, 2, or 3 CDR sequences selected from SEQ ID NO: 188, SEQ ID NO: 108, and SEQ ID NO: 37; or
y) a heavy chain variable region comprising 1, 2, or 3 CDR sequences selected from SEQ ID NO: 222, SEQ ID NO: 223, and SEQ ID NO: 224;
and a kappa light chain variable region comprising 1, 2, or 3 CDR sequences selected from SEQ ID NO: 225, SEQ ID NO: 226, and SEQ ID NO: 227.

78. The antibody or antigen-binding fragment thereof of claim 77, comprising:
a) a heavy chain variable region comprising SEQ ID NO: 50 and a light chain variable region comprising SEQ ID NO: 52;
b) a heavy chain variable region comprising SEQ ID NO: 54 and a light chain variable region comprising SEQ ID NO: 56;
c) a heavy chain variable region comprising SEQ ID NO: 58 and a light chain variable region comprising SEQ ID NO: 64;
d) a heavy chain variable region comprising SEQ ID NO: 62 and a light chain variable region comprising SEQ ID NO: 64;
e) a heavy chain variable region comprising SEQ ID NO: 66 and a light chain variable region comprising SEQ ID NO: 68;
f) a heavy chain variable region comprising SEQ ID NO: 70 and a light chain variable region comprising SEQ ID NO: 72;
g) a heavy chain variable region comprising SEQ ID NO: 74 and a light chain variable region comprising SEQ ID NO: 76;
h) a heavy chain variable region comprising SEQ ID NO: 78 and a light chain variable region comprising SEQ ID NO: 80;
i) a heavy chain variable region comprising SEQ ID NO: 82 and a light chain variable region comprising SEQ ID NO: 84;
j) a heavy chain variable region comprising SEQ ID NO: 86 and a light chain variable region comprising SEQ ID NO: 88;
k) a heavy chain variable region comprising SEQ ID NO: 101 and a light chain variable region comprising SEQ ID NO: 103;
l) a heavy chain variable region comprising SEQ ID NO: 109 and a light chain variable region comprising SEQ ID NO: 111;
m) a heavy chain variable region comprising SEQ ID NO: 118 and a light chain variable region comprising SEQ ID NO: 120 or SEQ ID NO: 221;
n) a heavy chain variable region comprising SEQ ID NO: 122 and a light chain variable region comprising SEQ ID NO: 124;
o) a heavy chain variable region comprising SEQ ID NO: 132 and a light chain variable region comprising SEQ ID NO: 134;
p) a heavy chain variable region comprising SEQ ID NO: 141 and a light chain variable region comprising SEQ ID NO: 143;
q) a heavy chain variable region comprising SEQ ID NO: 146 and a light chain variable region comprising SEQ ID NO: 148;
r) a heavy chain variable region comprising SEQ ID NO: 156 and a light chain variable region comprising SEQ ID NO: 158;
s) a heavy chain variable region comprising SEQ ID NO: 164 and a light chain variable region comprising SEQ ID NO: 166;
t) a heavy chain variable region comprising SEQ ID NO: 174 and a light chain variable region comprising SEQ ID NO: 176;
u) a heavy chain variable region comprising SEQ ID NO: 177 and a light chain variable region comprising SEQ ID NO: 180;
v) a heavy chain variable region comprising SEQ ID NO: 178 and a light chain variable region comprising SEQ ID NO: 180;
w) a heavy chain variable region comprising SEQ ID NO: 179 and a light chain variable region comprising SEQ ID NO: 180;
x) a heavy chain variable region comprising SEQ ID NO: 177 and a light chain variable region comprising SEQ ID NO: 181;
y) a heavy chain variable region comprising SEQ ID NO: 178 and a light chain variable region comprising SEQ ID NO: 181;
z) a heavy chain variable region comprising SEQ ID NO: 179 and a light chain variable region comprising SEQ ID NO: 181;
aa) a heavy chain variable region comprising SEQ ID NO: 177 and a light chain variable region comprising SEQ ID NO: 182;
bb) a heavy chain variable region comprising SEQ ID NO: 178 and a light chain variable region comprising SEQ ID NO: 182;
cc) a heavy chain variable region comprising SEQ ID NO: 179 and a light chain variable region comprising SEQ ID NO: 182;
dd) a heavy chain variable region comprising SEQ ID NO: 177 and a light chain variable region comprising SEQ ID NO: 183;
ee) a heavy chain variable region comprising SEQ ID NO: 178 and a light chain variable region comprising SEQ ID NO: 183;
ff) a heavy chain variable region comprising SEQ ID NO: 179 and a light chain variable region comprising SEQ ID NO: 183;
gg) a heavy chain variable region comprising SEQ ID NO: 189 and a light chain variable region comprising SEQ ID NO: 195;
hh) a heavy chain variable region comprising SEQ ID NO: 189 and a light chain variable region comprising SEQ ID NO: 196;
ii) a heavy chain variable region comprising SEQ ID NO: 189 and a light chain variable region comprising SEQ ID NO: 197;
jj) a heavy chain variable region comprising SEQ ID NO: 189 and a light chain variable region comprising SEQ ID NO: 198;
kk) a heavy chain variable region comprising SEQ ID NO: 189 and a light chain variable region comprising SEQ ID NO: 199;
ll) a heavy chain variable region comprising SEQ ID NO: 190 and a light chain variable region comprising SEQ ID NO: 200;
mm) a heavy chain variable region comprising SEQ ID NO: 190 and a light chain variable region comprising SEQ ID NO: 201;
nn) a heavy chain variable region comprising SEQ ID NO: 190 and a light chain variable region comprising SEQ ID NO: 202;
oo) a heavy chain variable region comprising SEQ ID NO: 190 and a light chain variable region comprising SEQ ID NO: 203;
pp) a heavy chain variable region comprising SEQ ID NO: 191 and a light chain variable region comprising SEQ ID NO: 200;
qq) a heavy chain variable region comprising SEQ ID NO: 191 and a light chain variable region comprising SEQ ID NO: 201;
rr) a heavy chain variable region comprising SEQ ID NO: 191 and a light chain variable region comprising SEQ ID NO: 202;
ss) a heavy chain variable region comprising SEQ ID NO: 191 and a light chain variable region comprising SEQ ID NO: 203;
tt) a heavy chain variable region comprising SEQ ID NO: 192 and a light chain variable region comprising SEQ ID NO: 200;
uu) a heavy chain variable region comprising SEQ ID NO: 192 and a light chain variable region comprising SEQ ID NO: 201;
vv) a heavy chain variable region comprising SEQ ID NO: 192 and a light chain variable region comprising SEQ ID NO: 202;
ww) a heavy chain variable region comprising SEQ ID NO: 192 and a light chain variable region comprising SEQ ID NO: 203;
xx) a heavy chain variable region comprising SEQ ID NO: 193 and a light chain variable region comprising SEQ ID NO: 200;
yy) a heavy chain variable region comprising SEQ ID NO: 193 and a light chain variable region comprising SEQ ID NO: 201;
zz) a heavy chain variable region comprising SEQ ID NO: 193 and a light chain variable region comprising SEQ ID NO: 202;
aaa) a heavy chain variable region comprising SEQ ID NO: 193 and a light chain variable region comprising SEQ ID NO: 203;
bbb) a heavy chain variable region comprising SEQ ID NO: 194 and a light chain variable region comprising SEQ ID NO: 200;
ccc) a heavy chain variable region comprising SEQ ID NO: 194 and a light chain variable region comprising SEQ ID NO: 201;
ddd) a heavy chain variable region comprising SEQ ID NO: 194 and a light chain variable region comprising SEQ ID NO: 202; or
eee) a heavy chain variable region comprising SEQ ID NO: 194 and a light chain variable region comprising SEQ ID NO: 203.

79. The antibody or antigen-binding fragment thereof of claim 77, which is bispecific.

80. The antibody or antigen-binding fragment thereof of claim 79, capable of specifically binding to a first and a second epitope of CD24, or capable of specifically binding to CD24 and a second antigen.

81. The antibody or antigen-binding fragment thereof of claim 80, which binds CD24 via a Fab and binds the second antigen via a scFv.

82. The antibody or antigen-binding fragment thereof of claim 81, the second antigen-binding scFv is operably linked to the C terminus of the heavy chain constant region following the CD24-binding Fab.

83. The antibody or antigen-binding fragment thereof of claim 82, the bispecific antibody molecule comprises a heavy chain in the format of: VH(anti-CD24)-CH1-Hinge-CH2-CH3-spacer-second antigen-binding scFv, associated with a light chain in the format of VL(anti-CD24)-CL.

84. The antibody or antigen-binding fragment thereof of claim 80, wherein the second antigen is an immune related target, optionally selected from the group consisting of: PD-L1, PD-L2, PD-1, CLTA-4, TIM-3, LAG3, CD160, 2B4, TGF s, VISTA, BTLA, TIGIT, LAIR1, OX40, CD2, CD27, ICAM-1, NKG2C, SLAMF7, NKp80, CD160, B7-H3, LFA-1, ICOS, 4-1BB, GITR, CD30, CD40, BAFFR, HVEM, CD7, LIGHT, IL-2, IL-15, CD3, CD16, SIRPα, Siglec 10, LILRB2, Clever-1, Macro, LILRB4, Siglec15, CSF1R, PSGL-1, VSIG-4, B2M and CD83.

85. The antibody or antigen-binding fragment thereof of claim 80, wherein the second antigen comprises CA-125, gangliosides G(D2), G(M2) and G(D3), CD19, CD20, CD33, CD47, CD52, Ep-CAM, CEA, CLDN18.2, bombesin-like peptides, PSA, HER2/neu, epidermal growth factor receptor (EGFR), erbB2, erbB3/HER3, erbB4, CD44v6, CD44v9, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R, CDH6, CEA, FOLR1, TROP2, PTK7, SLITRK6, CD142, NECTIN-4, ROR1, ROR2, CD142, CD123, CD22, CD79b, DLL3, SLC family or CO17-1A.

86. The antibody or antigen-binding fragment thereof of claim 80, comprises a heavy chain in the format of: VH(anti-CD24)-CH1-Hinge-CH2-CH3-spacer-scFv(anti-CD47), associated with a light chain in the format of VL(anti-CD24)-CL.

87. The antibody or antigen-binding fragment thereof of claim 77, wherein the antibody or antigen-binding fragment is linked to one or more conjugates, optionally, wherein the conjugate is covalently attached either directly or via a linker.

88. A pharmaceutical composition comprising the antibody or antigen-binding fragment thereof of claim 77, and a pharmaceutically acceptable carrier.

89. An isolated polynucleotide encoding the antibody or antigen-binding fragment thereof of claim 77.

90. A vector comprising the isolated polynucleotide of claim 89.

91. A host cell comprising the vector of claim 90.

92. A method of treating a disease or condition in a subject that would benefit from modulation of CD24 activity, comprising administering to the subject a therapeutically effective amount of the antibody or antigen-binding fragment thereof of claim 77.

93. A method of detecting presence or amount of CD24 in a sample, comprising contacting the sample with the antibody or antigen-binding fragment thereof of claim 77, and determining the presence or the amount of CD24 in the sample.

94. A chimeric antigen receptor (CAR) comprising an antigen binding domain, a transmembrane domain, a costimulatory signaling region, and a TCR signaling domain, wherein the antigen binding domain specifically binds to CD24 and comprises an antigen binding fragment of claim 77.

95. A method of treating cancer in a subject in need thereof, comprising administering an anti-CD24 antibody or antigen-binding fragment thereof that only binds to the glycosylated or sialylated CD24, preferably, only binds to the sialic acid of CD24.

96. The method of claim 95, wherein the anti-CD24 antibody or antigen-binding fragment thereof competes for the same epitope with the antibody or antigen-binding fragment thereof comprising: a heavy chain variable region comprising HCDR1, HCDR2, and HCDR3 of SEQ ID NO: 104, SEQ ID NO: 105, and SEQ ID NO: 106 respectively; and a light chain variable region comprising LCDR1, LCDR2, and LCDR3, wherein the LCDR1 comprises an amino acid sequence selected from the group consisting of SEQ ID NO: 107, 184, 185, 186, 187 and 188, the LCDR2 comprises an amino acid sequence of SEQ ID NO: 108, and the LCDR3 comprises an amino acid sequence of SEQ ID NO: 37, or comprising: a heavy chain variable region comprising HCDR1, HCDR2, and HCDR3 of SEQ ID NO: 95, SEQ ID NO: 96, and SEQ ID NO: 97 respectively;
and a light chain variable region comprising LCDR1, LCDR2, and LCDR3 of SEQ ID NO: 98, SEQ ID NO: 99, and SEQ ID NO: 100 respectively.

97. The method of claim 96, wherein the anti-CD24 antibody or antigen-binding fragment thereof comprises: a heavy chain variable region comprising HCDR1, HCDR2, and HCDR3 of SEQ ID NO: 104, SEQ ID NO: 105, and SEQ ID NO: 106 respectively; and a light chain variable region comprising LCDR1, LCDR2, and LCDR3, wherein the LCDR1 comprises an amino acid sequence selected from the group consisting of SEQ ID NO: 107, 184, 185, 186, 187 and 188, the LCDR2 comprises an amino acid sequence of SEQ ID NO: 108, and the LCDR3 comprises an amino acid sequence of SEQ ID NO: 37, or comprises: a heavy chain variable region comprising HCDR1, HCDR2, and HCDR3 of SEQ ID NO: 95, SEQ ID NO: 96, and SEQ ID NO: 97 respectively; and a light chain variable region comprising LCDR1, LCDR2, and LCDR3 of SEQ ID NO: 98, SEQ ID NO: 99, and SEQ ID NO: 100 respectively.
